SEQUENCE LISTING (56 sequences)

SEQ ID NO: 1 - protein, 360 aa, Homo sapiens
MNPTDIADTTLDESIYSNYYLYESIPKPCTKEGIKAFGELFLPPLYSLVFVFGLLGNSVVVLVLFKYKRLRSMTDVYLLNLAISDLLFVFSLPFWGYYAADQWVFGLGLCKMISWMYLVGFYSGIFFVMLMSIDRYLAIVHAVFSLRARTLTYGVITSLATWSVAVFASLPGFLFSTCYTERNHTYCKTKYSLNSTTWKVLSSLEINILGLVIPLGIMLFCYSMIIRTLQHCKNEKKNKAVKMIFAVVVLFLGFWTPYNIVLFLETLVELEVLQDCTFERYLDYAIQATETLAFVHCCLNPIIYFFLGEKFRKYILQLFKTCRGLFVLCQYCGLLQIYSADTPSSSYTQSTMDHDLHDAL

SEQ ID NO: 2 - protein, 374 aa, Homo sapiens
MSGESMNFSDVFDSSEDYFVSVNTSYYSVDSEMLLCSLQEVRQFSRLFVPIAYSLICVFGLLGNILVVITFAFYKKARSMTDVYLLNMAIADILFVLTLPFWAVSHATGAWVFSNATCKLLKGIYAINFNCGMLLLTCISMDRYIAIVQATKSFRLRSRTLPRSKIICLVVWGLSVIISSSTFVFNQKYNTQGSDVCEPKYQTVSEPIRWKLLMLGLELLFGFFIPLMFMIFCYTFIVKTLVQAQNSKRHKAIRVIIAVVLVFLACQIPHNMVLLVTAANLGKMNRSCQSEKLIGYTKTVTEVLAFLHCCLNPVLYAFIGQKFRNYFLKILKDLWCVRRKYKSSGFSCAGRYSENISRQTSETADNDNASSFTM

SEQ ID NO: 3 - protein, 352 aa, Homo sapiens
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 4 - protein, 378 aa, Homo sapiens
MDLGKPMKSVLVVALLVIFQVCLCQDEVTDDYIGDNTTVDYTLFESLCSKKDVRNFKAWFLPIMYSIICFVGLLGNGLVVLTYIYFKRLKTMTDTYLLNLAVADILFLLTLPFWAYSAAKSWVFGVHFCKLIFAIYKMSFFSGMLLLLCISIDRYVAIVQAVSAHRHRARVLLISKLSCVGIWILATVLSIPELLYSDLQRSSSEQAMRCSLITEHVEAFITIQVAQMVIGFLVPLLAMSFCYLVIIRTLLQARNFERNKAIKVIIAVVVVFIVFQLPYNGVVLAQTVANFNITSSTCELSKQLNIAYDVTYSLACVRCCVNPFLYAFIGVKFRNDLFKLFKDLGCLSQEQLRQWSSCRHIRRSSMSVEAETTTTFSP

SEQ ID NO: 5 - protein, 368 aa, Homo sapiens
MVLEVSDHQVLNDAEVAALLENFSSSYDYGENESDSCCTSPPCPQDFSLNFDRAFLPALYSLLFLLGLLGNGAVAAVLLSRRTALSSTDTFLLHLAVADTLLVLTLPLWAVDAAVQWVFGSGLCKVAGALFNINFYAGALLLACISFDRYLNIVHATQLYRRGPPARVTLTCLAVWGLCLLFALPDFIFLSAHHDERLNATHCQYNFPQVGRTALRVLQLVAGFLLPLLVMAYCYAHILAVLLVSRGQRRLRAMRLVVVVVVAFALCWTPYHLVVLVDILMDLGALARNCGRESRVDVAKSVTSGLGYMHCCLNPLLYAFVGVKFRERMWMLLLRLGCPNQRGLQRQPSSSRRDSSWSETSEASYSGL

SEQ ID NO: 6 - protein, 355 aa, Homo sapiens
MDQFPESVTENFEYDDLAEACYIGDIVVFGTVFLSIFYSVIFAIGLVGNLLVVFALTNSKKPKSVTDIYLLNLALSDLLFVATLPFWTHYLINEKGLHNAMCKFTTAFFFIGFFGSIFFITVISIDRYLAIVLAANSMNNRTVQHGVTISLGVWAAAILVAAPQFMFTKQKENECLGDYPEVLQEIWPVLRNVETNFLGFLLPLLIMSYCYFRIIQTLFSCKNHKKAKAIKLILLVVIVFFLFWTPYNVMIFLETLKLYDFFPSCDMRKDLRLALSVTETVAFSHCCLNPLIYAFAGEKFRRYLYHLYGKCLAVLCGRSVHVDFSSSESQRSRHGSVLSSNFTYHTSDGDALLLL

SEQ ID NO: 7 - DNA, 57 bp, synthetic construct
  source 1..57: mol_type = other DNA
gctactaacttcagcctgctgaagcaggctggagacgtggaggagaaccctggacct

SEQ ID NO: 8 - DNA, 54 bp, synthetic construct
  source 1..54: mol_type = other DNA
gagggcagaggaagtctgctaacatgcggtgacgtcgaggagaatcctggacct

SEQ ID NO: 9 - DNA, 60 bp, synthetic construct
  source 1..60: mol_type = other DNA
cagtgtactaattatgctctcttgaaattggctggagatgttgagagcaaccctggacct

SEQ ID NO: 10 - DNA, 66 bp, synthetic construct
  source 1..66: mol_type = other DNA
gtgaaacagactttgaattttgaccttctcaagttggcgggagacgtggagtccaaccctggacct

SEQ ID NO: 11 - DNA, 33 bp, synthetic construct
  source 1..33: mol_type = other DNA
aatacgactcactagtcgaggtgagccccacgt

SEQ ID NO: 12 - DNA, 23 bp, synthetic construct
  source 1..23: mol_type = other DNA
atggtggcagccgccggtcacac

SEQ ID NO: 13 - DNA, 23 bp, synthetic construct
  source 1..23: mol_type = other DNA
ggcggctgccaccatgagcgggg

SEQ ID NO: 14 - DNA, 34 bp, synthetic construct
  source 1..34: mol_type = other DNA
cacgggccccactagccatagagcccaccgcatc

SEQ ID NO: 15 - DNA, 22 bp, synthetic construct
  source 1..22: mol_type = other DNA
ccgatgcaaagtgccgataaac

SEQ ID NO: 16 - DNA, 22 bp, synthetic construct
  source 1..22: mol_type = other DNA
gcgctaatgctctgttacaggt

SEQ ID NO: 17 - DNA, 24 bp, synthetic construct
  source 1..24: mol_type = other DNA
gaggaagagttcttgcagctcggt

SEQ ID NO: 18 - DNA, 23 bp, synthetic construct
  source 1..23: mol_type = other DNA
ccgcagataacgacaatgcgtcg

SEQ ID NO: 19 - DNA, 25 bp, synthetic construct
  source 1..25: mol_type = other DNA
ccttgcgctaatgctctgttacagg

SEQ ID NO: 20 - DNA, 26 bp, synthetic construct
  source 1..26: mol_type = other DNA
cagaggcagggagtgggacaaaattg

SEQ ID NO: 21 - DNA, 20 bp, synthetic construct
  source 1..20: mol_type = other DNA
tgcccatggtgaaaacgggg

SEQ ID NO: 22 - DNA, 20 bp, synthetic construct
  source 1..20: mol_type = other DNA
ttgcccgcctgatgaatgct

SEQ ID NO: 23 - DNA, 21 bp, synthetic construct
  source 1..21: mol_type = other DNA
gcaatggcttctgcacaaaca

SEQ ID NO: 24 - DNA, 24 bp, synthetic construct
  source 1..24: mol_type = other DNA
gaggaagagttcttgcagctcggt

SEQ ID NO: 25 - DNA, 20 bp, synthetic construct
  source 1..20: mol_type = other DNA
ctggcgccaagcttctctgc

SEQ ID NO: 26 - DNA, 19 bp, synthetic construct
  source 1..19: mol_type = other DNA
accacaccggcgaagtcgt

SEQ ID NO: 27 - DNA, 29 bp, synthetic construct
  source 1..29: mol_type = other DNA
ggggctgcaggaattcgatatcaagcttc

SEQ ID NO: 28 - DNA, 23 bp, synthetic construct
  source 1..23: mol_type = other DNA
ctaggcctttcgctcaagttagt

SEQ ID NO: 29 - DNA, 987 bp, Homo sapiens
  source 1..987: mol_type = genomic DNA
atgctggtccgcaggggcgcgcgcgcagggcccaggatgccgcggggctggaccgcgctttgcttgctgagtttgctgccttctgggttcatgagtcttgacaacaacggtactgctaccccagagttacctacccagggaacattttcaaatgtttctacaaatgtatcctaccaagaaactacaacacctagtacccttggaagtaccagcctgcaccctgtgtctcaacatggcaatgaggccacaacaaacatcacagaaacgacagtcaaattcacatctacctctgtgataacctcagtttatggaaacacaaactcttctgtccagtcacagacctctgtaatcagcacagtgttcaccaccccagccaacgtttcaactccagagacaaccttgaagcctagcctgtcacctggaaatgtttcagacctttcaaccactagcactagccttgcaacatctcccactaaaccctatacatcatcttctcctatcctaagtgacatcaaggcagaaatcaaatgttcaggcatcagagaagtgaaattgactcagggcatctgcctggagcaaaataagacctccagctgtgcggagtttaagaaggacaggggagagggcctggcccgagtgctgtgtggggaggagcaggctgatgctgatgctggggcccaggtatgctccctgctccttgcccagtctgaggtgaggcctcagtgtctactgctggtcttggccaacagaacagaaatttccagcaaactccaacttatgaaaaagcaccaatctgacctgaaaaagctggggatcctagatttcactgagcaagatgttgcaagccaccagagctattcccaaaagaccctgattgcactggtcacctcgggagccctgctggctgtcttgggcatcactggctatttcctgatgaatcgccgcagctggagccccacaggagaaaggctgggcgaagacccttga

SEQ ID NO: 30 - DNA, 825 bp, Homo sapiens
  source 1..825: mol_type = genomic DNA
accccagagttacctacccagggaacattttcaaatgtttctacaaatgtatcctaccaagaaactacaacacctagtacccttggaagtaccagcctgcaccctgtgtctcaacatggcaatgaggccacaacaaacatcacagaaacgacagtcaaattcacatctacctctgtgataacctcagtttatggaaacacaaactcttctgtccagtcacagacctctgtaatcagcacagtgttcaccaccccagccaacgtttcaactccagagacaaccttgaagcctagcctgtcacctggaaatgtttcagacctttcaaccactagcactagccttgcaacatctcccactaaaccctatacatcatcttctcctatcctaagtgacatcaaggcagaaatcaaatgttcaggcatcagagaagtgaaattgactcagggcatctgcctggagcaaaataagacctccagctgtgcggagtttaagaaggacaggggagagggcctggcccgagtgctgtgtggggaggagcaggctgatgctgatgctggggcccaggtatgctccctgctccttgcccagtctgaggtgaggcctcagtgtctactgctggtcttggccaacagaacagaaatttccagcaaactccaacttatgaaaaagcaccaatctgacctgaaaaagctggggatcctagatttcactgagcaagatgttgcaagccaccagagctattcccaaaagaccctgattgcactggtcacctcgggagccctgctggctgtcttgggcatcactggctatttcctgatgaatcgc

SEQ ID NO: 31 - DNA, 117 bp, Homo sapiens
  source 1..117: mol_type = genomic DNA
atgctggtccgcaggggcgcgcgcgcagggcccaggatgccgcggggctggaccgcgctttgcttgctgagtttgctgccttctgggttcatgagtcttgacaacaacggtactgct

SEQ ID NO: 32 - DNA, 45 bp, Homo sapiens
  source 1..45: mol_type = genomic DNA
cgcagctggagccccacaggagaaaggctgggcgaagacccttga

SEQ ID NO: 33 - DNA, 1080 bp, Homo sapiens
  source 1..1080: mol_type = genomic DNA
atgaaccccacggatatagcagacaccaccctcgatgaaagcatatacagcaattactatctgtatgaaagtatccccaagccttgcaccaaagaaggcatcaaggcatttggggagctcttcctgcccccactgtattccttggtttttgtatttggtctgcttggaaattctgtggtggttctggtcctgttcaaatacaagcggctcaggtccatgactgatgtgtacctgctcaaccttgccatctcggatctgctcttcgtgttttccctccctttttggggctactatgcagcagaccagtgggtttttgggctaggtctgtgcaagatgatttcctggatgtacttggtgggcttttacagtggcatattctttgtcatgctcatgagcattgatagatacctggcaattgtgcacgcggtgttttccttgagggcaaggaccttgacttatggggtcatcaccagtttggctacatggtcagtggctgtgttcgcctcccttcctggctttctgttcagcacttgttatactgagcgcaaccatacctactgcaaaaccaagtactctctcaactccacgacgtggaaggttctcagctccctggaaatcaacattctcggattggtgatccccttagggatcatgctgttttgctactccatgatcatcaggaccttgcagcattgtaaaaatgagaagaagaacaaggcggtgaagatgatctttgccgtggtggtcctcttccttgggttctggacaccttacaacatagtgctcttcctagagaccctggtggagctagaagtccttcaggactgcacctttgaaagatacttggactatgccatccaggccacagaaactctggcttttgttcactgctgccttaatcccatcatctacttttttctgggggagaaatttcgcaagtacatcctacagctcttcaaaacctgcaggggcctttttgtgctctgccaatactgtgggctcctccaaatttactctgctgacacccccagctcatcttacacgcagtccaccatggatcatgatctccatgatgctctg

SEQ ID NO: 34 - DNA, 1122 bp, Homo sapiens
  source 1..1122: mol_type = genomic DNA
atgagcggggaatcaatgaatttcagcgatgttttcgactccagtgaagattattttgtgtcagtcaatacttcatattactcagttgattctgagatgttactgtgctccttgcaggaggtcaggcagttctccaggctatttgtaccgattgcctactccttgatctgtgtctttggcctcctggggaatattctggtggtgatcacctttgctttttataagaaggccaggtctatgacagacgtctatctcttgaacatggccattgcagacatcctctttgttcttactctcccattctgggcagtgagtcatgccaccggtgcgtgggttttcagcaatgccacgtgcaagttgctaaaaggcatctatgccatcaactttaactgcgggatgctgctcctgacttgcattagcatggaccggtacatcgccattgtacaggcgactaagtcattccggctccgatccagaacactaccgcgcagcaaaatcatctgccttgttgtgtgggggctgtcagtcatcatctccagctcaacttttgtcttcaaccaaaaatacaacacccaaggcagcgatgtctgtgaacccaagtaccagactgtctcggagcccatcaggtggaagctgctgatgttggggcttgagctactctttggtttctttatccctttgatgttcatgatattttgttacacgttcattgtcaaaaccttggtgcaagctcagaattctaaaaggcacaaagccatccgtgtaatcatagctgtggtgcttgtgtttctggcttgtcagattcctcataacatggtcctgcttgtgacggctgcaaatttgggtaaaatgaaccgatcctgccagagcgaaaagctaattggctatacgaaaactgtcacagaagtcctggctttcctgcactgctgcctgaaccctgtgctctacgcttttattgggcagaagttcagaaactactttctgaagatcttgaaggacctgtggtgtgtgagaaggaagtacaagtcctcaggcttctcctgtgccgggaggtactcagaaaacatttctcggcagaccagtgagaccgcagataacgacaatgcgtcgtccttcactatg

SEQ ID NO: 35 - DNA, 1128 bp, Homo sapiens
  source 1..1128: mol_type = genomic DNA
atggagcgcgcgtcctgcttgttgctgctgctgctgccgctggtgcacgtctctgcgaccacgccagaaccttgtgagctggacgatgaagatttccgctgcgtctgcaacttctccgaacctcagcccgactggtccgaagccttccagtgtgtgtctgcagtagaggtggagatccatgccggcggtctcaacctagagccgtttctaaagcgcgtcgatgcggacgccgacccgcggcagtatgctgacacggtcaaggctctccgcgtgcggcggctcacagtgggagccgcacaggttcctgctcagctactggtaggcgccctgcgtgtgctagcgtactcccgcctcaaggaactgacgctcgaggacctaaagataaccggcaccatgcctccgctgcctctggaagccacaggacttgcactttccagcttgcgcctacgcaacgtgtcgtgggcgacagggcgttcttggctcgccgagctgcagcagtggctcaagccaggcctcaaggtactgagcattgcccaagcacactcgcctgccttttcctgcgaacaggttcgcgccttcccggcccttaccagcctagacctgtctgacaatcctggactgggcgaacgcggactgatggcggctctctgtccccacaagttcccggccatccagaatctagcgctgcgcaacacaggaatggagacgcccacaggcgtgtgcgccgcactggcggcggcaggtgtgcagccccacagcctagacctcagccacaactcgctgcgcgccaccgtaaaccctagcgctccgagatgcatgtggtccagcgccctgaactccctcaatctgtcgttcgctgggctggaacaggtgcctaaaggactgccagccaagctcagagtgctcgatctcagctgcaacagactgaacagggcgccgcagcctgacgagctgcccgaggtggataacctgacactggacgggaatcccttcctggtccctggaactgccctcccccacgagggctcaatgaactccggcgtggtcccagcctgtgcacgttcgaccctgtcggtgggggtgtcgggaaccctggtgctgctccaaggggcccggggctttgcctaa

SEQ ID NO: 36 - DNA, 642 bp, Homo sapiens
  source 1..642: mol_type = genomic DNA
atgcctccgctgcctctggaagccacaggacttgcactttccagcttgcgcctacgcaacgtgtcgtgggcgacagggcgttcttggctcgccgagctgcagcagtggctcaagccaggcctcaaggtactgagcattgcccaagcacactcgcctgccttttcctgcgaacaggttcgcgccttcccggcccttaccagcctagacctgtctgacaatcctggactgggcgaacgcggactgatggcggctctctgtccccacaagttcccggccatccagaatctagcgctgcgcaacacaggaatggagacgcccacaggcgtgtgcgccgcactggcggcggcaggtgtgcagccccacagcctagacctcagccacaactcgctgcgcgccaccgtaaaccctagcgctccgagatgcatgtggtccagcgccctgaactccctcaatctgtcgttcgctgggctggaacaggtgcctaaaggactgccagccaagctcagagtgctcgatctcagctgcaacagactgaacagggcgccgcagcctgacgagctgcccgaggtggataacctgacactggacgggaatcccttcctggtccctggaactgccctcccccacgagggctcaatgaac

SEQ ID NO: 37 - DNA, 1671 bp, Homo sapiens
  source 1..1671: mol_type = genomic DNA
atgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattcttcaaagtgacgcctcccccaggaagcgggccccagaaccagtacgggaacgtgctgtctctccccacacccacctcaggcctcggacgcgcccagcgttgggccgcaggcctggggggcactgccccgtcttatggaaacccgagcagcgacgtccaggcggatggagccttggggtcccggagcccgccgggagtgggcccagaagaagaggaaggggagggctatgaggaacctgacagtgaggaggactccgagttctatgagaacgactccaaccttgggcaggaccagctctcccaggatggcagcggctacgagaaccctgaggatgagcccctgggtcctgaggatgaagactccttctccaacgctgagtcttatgagaacgaggatgaagagctgacccagccggtcgccaggacaatggacttcctgagccctcatgggtcagcctgggaccccagccgggaagcaacctccctggggtcccagtcctatgaggatatgagaggaatcctgtatgcagccccccagctccgctccattcggggccagcctggacccaatcatgaggaagatgcagactcttatgagaacatggataatcccgatgggccagacccagcctggggaggagggggccgcatgggcacctggagcaccaggtga

SEQ ID NO: 38 - DNA, 990 bp, Homo sapiens
  source 1..990: mol_type = genomic DNA
atgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccacc

SEQ ID NO: 39 - DNA, 894 bp, Homo sapiens
  source 1..894: mol_type = genomic DNA
atgacaacacccagaaattcagtaaatgggactttcccggcagagccaatgaaaggccctattgctatgcaatctggtccaaaaccactcttcaggaggatgtcttcactggtgggccccacgcaaagcttcttcatgagggaatctaagactttgggggctgtccagattatgaatgggctcttccacattgccctggggggtcttctgatgatcccagcagggatctatgcacccatctgtgtgactgtgtggtaccctctctggggaggcattatgtatattatttccggatcactcctggcagcaacggagaaaaactccaggaagtgtttggtcaaaggaaaaatgataatgaattcattgagcctctttgctgccatttctggaatgattctttcaatcatggacatacttaatattaaaatttcccattttttaaaaatggagagtctgaattttattagagctcacacaccatatattaacatatacaactgtgaaccagctaatccctctgagaaaaactccccatctacccaatactgttacagcatacaatctctgttcttgggcattttgtcagtgatgctgatctttgccttcttccaggaacttgtaatagctggcatcgttgagaatgaatggaaaagaacgtgctccagacccaaatctaacatagttctcctgtcagcagaagaaaaaaaagaacagactattgaaataaaagaagaagtggttgggctaactgaaacatcttcccaaccaaagaatgaagaagacattgaaattattccaatccaagaagaggaagaagaagaaacagagacgaactttccagaacctccccaagatcaggaatcctcaccaatagaaaatgacagctctccttaa

SEQ ID NO: 40 - DNA, 322 bp, Homo sapiens
  source 1..322: mol_type = genomic DNA
aagcttgccacccctgagcctgttcgccgccatctccggcatgatcctgagcatcatggacatcctgaacatcaagatcagccactttctgaagatggaaagcctgaacttcatccgggcccacaccccctacatcaacatctacaactgcgagcccgccaaccccagcgagaagaactcccccagcacccagtactgctacagcatccagagcctgtttctgggcatcctgagcgtgatgctgatcttcgccttctttcaggaactggtcatcgccgtgagcggctggcggctgttcaagaagattagctaatgaggatcc

SEQ ID NO: 41 - DNA, 834 bp, Homo sapiens
  source 1..834: mol_type = genomic DNA
atcgccatgcagagcggccccaagcccctgttccggcggatgagcagcctggtgggccccacacagagctttttcatgcgggagagcaagaccctgggcgccgtgcagatcatgaacggcctgttccacatcgccctgggcggcctgctgatgatccctgccggcatctacgcccccatctgcgtgaccgtgtggtatcccctgtggggcggcatcatgtacatcatcagcggcagcctgctggccgccaccgagaagaacagccggaagtgcctggtgaagggcaagatgatcatgaactccctgagcctgttcgccgccatctccggcatgatcctgagcatcatggacatcctgaacatcaagatcagccactttctgaagatggaaagcctgaacttcatccgggcccacaccccctacatcaacatctacaactgcgagcccgccaaccccagcgagaagaactcccccagcacccagtactgctacagcatccagagcctgtttctgggcatcctgagcgtgatgctgatcttcgccttctttcaggaactggtcatcgccggcatcgtggagaacgagtggaagcggacctgcagccggcccaagagcaacatcgtgctgctgtccgccgaggaaaagaaagagcagaccatcgagatcaaggaagaggtggtgggcctgaccgagaccagctcccagcccaagaacgaagaggatatcgagatcatcccaatccaggaagaggaagaagaagagaccgagacaaacttccccgagcccccccaggaccaggaaagcagccccatcgagaacgacagcagcccctga

SEQ ID NO: 42 - DNA, 618 bp, Homo sapiens
  source 1..618: mol_type = genomic DNA
atggcgcacgctgggagaacagggtacgataaccgggagatagtgatgaagtacatccattataagctgtcgcagaggggctacgagtgggatgcgggagatgtgggcgccgcgcccccgggggccgcccccgcaccgggcatcttctcctcccagcccgggcacacgccccatccagccgcatcccgggacccggtcgccaggacctcgccgctgcagaccccggctgcccccggcgccgccgcggggcctgcgctcagcccggtgccacctgtggtccacctgaccctccgccaggccggcgacgacttctcccgccgctaccgccgcgacttcgccgagatgtccagccagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggtaggtgcacttggtgatgtgagtctgggctga

SEQ ID NO: 43 - DNA, 1050 bp, Homo sapiens
  source 1..1050: mol_type = genomic DNA
atgtttggcctcaaaagaaacgcggtaatcggactcaacctctactgtgggggggccggcttgggggccggcagcggcggcgccacccgcccgggagggcgacttttggctacggagaaggaggcctcggcccggcgagagatagggggaggggaggccggcgcggtgattggcggaagcgccggcgcaagccccccgtccaccctcacgccagactcccggagggtcgcgcggccgccgcccattggcgccgaggtccccgacgtcaccgcgacccccgcgaggctgcttttcttcgcgcccacccgccgcgcggcgccgcttgaggagatggaagccccggccgctgacgccatcatgtcgcccgaagaggagctggacgggtacgagccggagcctctcgggaagcggccggctgtcctgccgctgctggagttggtcggggaatctggtaataacaccagtacggacgggtcactaccctcgacgccgccgccagcagaggaggaggaggacgagttgtaccggcagtcgctggagattatctctcggtaccttcgggagcaggccaccggcgccaaggacacaaagccaatgggcaggtctggggccaccagcaggaaggcgctggagaccttacgacgggttggggatggcgtgcagcgcaaccacgagacggccttccaaggcatgcttcggaaactggacatcaaaaacgaagacgatgtgaaatcgttgtctcgagtgatgatccatgttttcagcgacggcgtaacaaactggggcaggattgtgactctcatttcttttggtgcctttgtggctaaacacttgaagaccataaaccaagaaagctgcatcgaaccattagcagaaagtatcacagacgttctcgtaaggacaaaacgggactggctagttaaacaaagaggctgggatgggtttgtggagttcttccatgtagaggacctagaaggtggcatcaggaatgtgctgctggcttttgcaggtgttgctggagtaggagctggtttggcatatctaataaga

SEQ ID NO: 44 - DNA, 681 bp, Homo sapiens
  source 1..681: mol_type = genomic DNA
ggtaccgccaccatggacgggtccggggagcagcccagaggcggggggcccaccagctctgagcagatcatgaagacaggggcccttttgcttcagggtttcatccaggatcgagcagggcgaatggggggggaggcacccgagctggccctggacccggtgcctcaggatgcgtccaccaagaagctgagcgagtgtctcaagcgcatcggggacgaactggacagtaacatggagctgcagaggatgattgccgccgtggacacagactccccccgagaggtctttttccgagtggcagctgacatgttttctgacggcaacttcaactggggccgggttgtcgcccttttctactttgccagcaaactggtgctcaaggccctgtgcaccaaggtgccggaactgatcagaaccatcatgggctggacattggacttcctccgggagcggctgttgggctggatccaagaccagggtggttgggggctgcccctggccgagtcactgaagcgactgatgtccctgtctccaggacggcctcctctcctactttgggacgcccacgtggcagaccgtgaccatctttgtggcgggagtgctcaccgcctcactcaccatctggaagaagatgggctgaggcccccagctgccttggactgtgtttttcctccatctaga

SEQ ID NO: 45 - DNA, 354 bp, Homo sapiens
  source 1..354: mol_type = genomic DNA
ggtaccgccaccatggcaaagcaaccttctgatgtaagttctgagtgtgaccgagaaggtagacaattgcagcctgcggagaggcctccccagctcagacctggggcccctacctccctacagacagagccacaagacaggagcccagcacccatgagttgtgacaaatcaacacaaaccccaagtcctccttgccaggccttcaaccactatctcagtgcaatggtagtcatcctagaggatataggtgatctttcactgtgctttggatttatatttactggcttagatttgtatggccaccaccatagtcaagatacagaacaactcaaccacaaggatttctcatctaga

SEQ ID NO: 46 - DNA, 723 bp, Homo sapiens
  source 1..723: mol_type = genomic DNA
atgtgcagcggtgctggggtcatgatggctcggtgggcagcgaggggccgggccggctggaggagcacagtgcggattctgtcgccactgggacactgtgaaccaggagtgagtcggagctgccgcgctgcccaggccatggactgtgaggtcaacaacggttccagcctcagggatgagtgcatcacaaacctactggtgtttggcttcctccaaagctgttctgacaacagcttccgcagagagctggacgcactgggccacgagctgccagtgctggctccccagtgggagggctacgatgagctgcagactgatggcaaccgcagcagccactcccgcttgggaagaatagaggcagattctgaaagtcaagaagacatcatccggaatattgccaggcacctcgcccaggtcggggacagcatggaccgtagcatccctccgggcctggtgaacggcctggccctgcagctcaggaacaccagccggtcggaggaggaccggaacagggacctggccactgccctggagcagctgctgcaggcctaccctagagacatggagaaggagaagaccatgctggtgctggccctgctgctggccaagaaggtggccagtcacacgccgtccttgctccgtgatgtctttcacacaacagtgaattttattaaccagaacctacgcacctacgtgaggagcttagccagaaatgggatggac

SEQ ID NO: 47 - DNA, 162 bp, Homo sapiens
  source 1..162: mol_type = genomic DNA
atgcctgggaagaaggcgcgcaagaacgctcaaccgagccccgcgcgggctccagcagagctggaagtcgagtgtgctactcaactcaggagatttggagacaaactgaacttccggcagaaacttctgaatctgatatccaaactcttctgctcaggaacc

SEQ ID NO: 48 - DNA, 582 bp, Homo sapiens
  source 1..582: mol_type = genomic DNA
atggcccgcgcacgccaggagggcagctccccggagcccgtagagggcctggcccgcgacggcccgcgccccttcccgctcggccgcctggtgccctcggcagtgtcctgcggcctctgcgagcccggcctggctgccgcccccgccgcccccaccctgctgcccgctgcctacctctgcgcccccaccgccccacccgccgtcaccgccgccctggggggttcccgctggcctgggggtccccgcagccggccccgaggcccgcgcccggacggtcctcagccctcgctctcgctggcggagcagcacctggagtcgcccgtgcccagcgccccgggggctctggcgggcggtcccacccaggcggccccgggagtccgcggggaggaggaacagtgggcccgggagatcggggcccagctgcggcggatggcggacgacctcaacgcacagtacgagcggcggagacaagaggagcagcagcggcaccgcccctcaccctggagggtcctgtacaatctcatcatgggactcctgcccttacccaggggccacagagcccccgagatggagcccaattag

SEQ ID NO: 49 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 50 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 51 - protein, 219 aa, Homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 52 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 53 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 54 - protein, 193 aa, Homo sapiens
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 55 - protein, 162 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 56 - protein, 188 aa, Homo sapiens
MALTFALLVALLVLSCKSSCSVGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

(19) United States
(12) Patent Application Publication
(10) Pub. No.: US 2025/0179149 A1
(43) Pub. Date: Jun. 5, 2025

(54) Use Of Chemokine Receptors In Cellular Homing

(71) Applicant: CarryGenes Bioengineering, LLC, Savannah, GA (US)

(72) Inventors: Edward L. PERKINS, Savannah, GA (US); Amy L. GREENE, Savannah, GA (US); Dominique BROCCOLI, Savannah, GA (US); Kara Pascarelli MANNE, Savannah, GA (US); Ola WINQVIST, Stockholm (SE); John ANDERSSON, Stockholm (SE); Marton KESZEI, Stockholm (SE); Katarina LYBERG, Stockholm (SE); Maria EKOFF, Stockholm (SE); Julia REMNESTÅL, Stockholm (SE)

(73) Assignee: CarryGenes Bioengineering, LLC, Savannah, GA (US)

(21) Appl. No.: 18/685,905

(22) PCT Filed: Aug. 26, 2022
(86) PCT No.: PCT/US2022/075520

§ 371 Date: Feb. 23, 2024

Related U.S. Application Data
(60) Provisional application No. 63/238,736, filed on Aug. 30, 2021.

(51) Int. Cl. C07K 14/715; A61K 40/11; A61K 40/42; C12N 5/0783

(52) U.S. Cl. CPC C07K 14/7158; A61K 40/11; A61K 40/4219; C12N 5/0636; C12N 2510/00

(57) ABSTRACT

Aspects of the present invention relate to the use of chemokine receptors such as e.g., CCR4 and CCR6, as homing agents. The chemokine receptor(s) is/are expressed by cells containing synthetic chromosomes. The chemokine receptor(s) is/are under controllable expression from a synthetic chromosome and can be used to control an immune response so as to treat or inhibit a disease such as cancer. The control is provided by inducing leukocyte trafficking to sites of disease. If more than one chemokine receptor is expressed, the levels can be individually controlled so that the desired cellular trafficking pattern can be obtained.

### FIELD

The present invention relates to the use of chemokine receptors such as e.g., CCR4 and CCR6, as homing means. The chemokine receptor(s) is/are expressed by cells containing synthetic chromosomes. The chemokine receptor(s) is/are under controllable expression from a synthetic chromosome. An aim of the invention is to control an immune response to treat a disease such as cancer. The control is provided by inducing leukocyte trafficking to sites of disease. If more than one chemokine receptor is expressed the levels can be individually controlled so that the desired cellular trafficking pattern can be obtained.

### BACKGROUND

Chemokine receptors are key regulators of leukocyte trafficking and consequently also of immunity. They have therefore attracted substantial interest as therapeutic targets in both inflammatory diseases and cancer. In the context of cancer, single chemokine receptors have been used to facilitate cell therapies. A current challenge for using chemokine receptors in immunotherapy is that the site of disease in cancer changes over time when a tumor metastasizes.

### SUMMARY OF THE INVENTION

Specific embodiments of the invention appear from the appended claims, wherein
1. A synthetic chromosome comprising a nucleic acid sequence encoding a chemokine receptor.
2. A synthetic chromosome according to claim 1, wherein the chemokine receptor is selected from CCR4, CCR6, CXCR4, CCR7, CXCR3, and CX3CR1.
3. A synthetic chromosome according to claim 1 or 2, wherein the chemokine receptor is CCR4 or CCR6.
4. A synthetic chromosome according to any one of the preceding claims, wherein the chromosome comprises two or more nucleic acid sequences encoding two or more chemokine receptors, wherein the two or more chemokine receptors are the same or different.
5. A synthetic chromosome according to claim 4, wherein the nucleic acid sequences encode CCR4 and CCR6, respectively.
5. A synthetic chromosome according to any one of the preceding claims comprising one or more promoters independently controlling expression of one or more chemokine receptors.
6. A synthetic chromosome according to any one of the preceding claims, wherein one or more promoters are inducible.
7. A synthetic chromosome according to claim 5 or 6, wherein one or more promoters are constitutive.
8. A synthetic chromosome according to any one of claims 5-7, comprising two or more promoters, one of which is inducible and another is constitutive.
9. A synthetic chromosome according to any one of claims 5, 6, 8, wherein the chemokine receptor is CCR6 and expression of CCR6 is inducible.
10. A synthetic chromosome according to any one of claims 5, 7, 8, wherein the chemokine receptor is CCR4 and expression of CCR4 is constitutive.
11. A synthetic chromosome according to any one of the preceding claims comprising one or more insulators.
12. A synthetic chromosome according to any one of the preceding claims for use in cellular homing.
13. A synthetic chromosome for use in enhancing an therapeutic response in or in the vicinity of a target tissue by providing chemokine receptors expressed by cells carrying the chromosome.
14. A cell comprising a synthetic chromosome as defined in any one of the preceding claims.
15. A cell according to claim 13, wherein the cell is a leukocyte.
16. A cell according to any of the preceding claims for medical use, veterinary use, or diagnostic use.
17. A cell comprising a synthetic chromosome as defined in any one of claims 1-13 for use in enhancing a response in or in the vicinity of a target tissue by providing chemokine receptors.
18. A composition comprising a synthetic chromosome as defined in any one of claim 1-13 and an additive.
19. A composition comprising a cell as defined in any one of claim 14-17 and an additive.

### DETAILED DESCRIPTION

Herein is presented a synthetic chromosome-derived invention used to express multiple chemokine receptors allowing them to traffic different tumor sites. In general, leukocytes such as tumor-specific T cells are transfected with a synthetic chromosome, that encodes for the chemokine receptor(s) of choice. The leukocytes may also be lymphocytes, tumor-infiltrating lymphocytes, T cells, NK cells, B cells or other leucocyte subsets with anti-tumoral activity. The chemokine receptors can be under constitutive or inducible genetic control, allowing for differential trafficking.

Determination of whether the expression should be constitutive or inducible depends on the chemokine receptor in question, the target tissue and the type of leukocyte in which the chemokine receptor is expressed. An example of the use of a combination of constitutive and inducible expression is described herein using the chemokine receptors CCR4 and CCR6 as examples.

The methods described herein may employ, unless otherwise indicated, conventional techniques and descriptions of molecular biology (including recombinant techniques), cell biology, biochemistry, and cellular engineering technology, all of which are within the skill of those who practice in the art. Such conventional techniques include oligonucleotide synthesis, hybridization and ligation of oligonucleotides, transformation and transduction of cells, engineering of recombination systems, creation of transgenic animals and plants, and human gene therapy. Specific illustrations of suitable techniques can be had by reference to the examples herein. However, equivalent conventional procedures can, of course, also be used. Such conventional techniques and descriptions can be found in standard laboratory manuals such as Genome Analysis: A Laboratory Manual Series (Vols. I-IV) (Green, et al., eds., 1999); Genetic Variation: A Laboratory Manual (Weiner, et al., eds., 2007); Sambrook and Russell, Condensed Protocols from Molecular Cloning: A Laboratory Manual (2006); and Sambrook and Russell, Molecular Cloning: A Laboratory Manual (2002) (all from Cold Spring Harbor Laboratory Press); Protein Methods (Bollag et al., John Wiley & Sons 1996); Nonviral Vectors for Gene Therapy (Wagner et al. eds., Academic Press 1999); Viral Vectors (Kaplift & Loewy, eds., Academic Press 1995); Immunology Methods Manual (Lefkovits ed., Academic Press 1997); Gene Therapy Techniques, Applications and Regulations From Laboratory to Clinic (Meager, ed., John Wiley & Sons 1999); M. Giacca, Gene Therapy (Springer 2010); Gene Therapy Protocols (LeDoux, ed., Springer 2008); Cell and Tissue Culture: Laboratory Procedures in Biotechnology (Doyle & Griffiths, eds., John Wiley & Sons 1998); Mammalian Chromosome Engineering—Methods and Protocols (G. Hadlaczky, ed., Humana Press 2011); Essential Stem Cell Methods, (Lanza and Klimanskaya, eds., Academic Press 2011); Stem Cell Therapies: Opportunities for Ensuring the Quality and Safety of Clinical Offerings: Summary of a Joint Workshop (Board on Health Sciences Policy, National Academies Press 2014); Essentials of Stem Cell Biology, Third Ed., (Lanza and Atala, eds., Academic Press 2013); FISH protocol (Molecular Cytogenetics: Protocols and Applications (Y-S Fan ed. Meth Molecular Bio Series, Vol 204, Human Press, 2002) and Handbook of Stem Cells, (Atala and Lanza, eds., Academic Press 2012), all of which are herein incorporated by reference in their entirety for all purposes. Before the present compositions, research tools and methods are described, it is to be understood that this invention is not limited to the specific methods, compositions, targets and uses described, as such may, of course, vary. It is also to be understood that the terminology used herein is for the purpose of describing particular aspects only and is not intended to limit the scope of the present invention, which will be limited only by the appended claims.

Note that as used in the present specification and in the appended claims, the singular forms “a,” “and,” and “the” include plural referents unless the context clearly dictates otherwise. Thus, for example, reference to “a composition” refers to one or mixtures of compositions, and reference to “an assay” includes reference to equivalent steps and methods known to those skilled in the art, and so forth.

Unless defined otherwise, all technical and scientific terms used herein have the same meaning as commonly understood by one of ordinary skill in the art to which this invention belongs. All publications mentioned herein are incorporated herein by reference for the purpose of describing and disclosing devices, formulations and methodologies which are described in the publication, and which might be used in connection with the presently described invention.

Where a range of values is provided, it is understood that each intervening value between the upper and lower limit of that range and any other stated or intervening value in that stated range is encompassed within the invention. The upper and lower limits of these smaller ranges may independently be included in the smaller ranges, subject to any specifically excluded limit in the stated range. Where the stated range includes both of the limits, ranges excluding only one of those included limits are also included in the invention.

In the following description, numerous specific details are set forth to provide a more thorough understanding of the present invention. However, it will be apparent to one of ordinary skill in the art upon reading the specification that the present invention may be practiced without one or more of these specific details. In other instances, well-known features and procedures well known to those skilled in the art have not been described in order to avoid obscuring the invention.

Chemokines and their Receptors

Chemokines are chemotactic cytokines, i.e., signaling molecules of the immune system, that signal through cell surface G protein-coupled heptahelical chemokine receptors. Their main function is to stimulate the migration of cells including leukocytes. Tumors also require specific homing receptors to metastasize into their characteristic organs. In colorectal cancer, for example, the liver is the predominant organ for distant metastasis. The chemokine receptor CCR6 is upregulated in colorectal cancer and its chemokine ligand CCL20 is predominantly expressed in lymphatic tissue and liver. Currently, it is impossible to alter the expression of chemokine receptors in transfected leukocytes over time, so they match the migratory pattern of cancer cells. The vectors in use today simply do not have enough space to carry several chemokine receptors, their regulatory elements and insulators. In contrast, the synthetic chromosome-based strategy described herein comes with several advantages:
1. The synthetic chromosome can carry up to 80 large genetic inserts. This allows for expression of multiple chemokine receptors.
2. The synthetic chromosome can carry large (>100 kilo base pairs) promoter and regulatory elements. This allows for fine tuning of the amount of constitutive and inducible chemokine receptor expression levels allowing for controlled cellular trafficking.

In describing the invention T cells and cancer are used as non-limiting examples. However, a person skilled in the art will understand that other cells than T cells can be used in the present context of using chemokine receptors to direct cellular trafficking. In the same manner other diseases than cancer may be treated as described herein.

The invention uses the space available on a synthetic chromosome (such as, e.g., hSync) for constitutive expression of a chemokine receptor that facilitates tumor-homing. In addition, the synthetic chromosome (e.g., hSync) carries an inducible chemokine receptor promoting trafficking to organs at risk for early metastasis events. Thus, if required, it is possible to move the synthetic chromosome (e.g., hSync) transfected cells from the original tumor to sites of metastasis.

### Chemokine Receptors

Chemokines are chemotactic peptides that mediate the migration and tissue infiltration of leukocytes. Chemokines bind to chemokine receptors which are seven-transmembrane domain G protein-coupled receptors (GPCRs). There are nearly 50 different human chemokines and 18 chemotactic chemokine receptors. Although chemokine receptors play a key role in healthy tissue immune homeostasis, their main task is to mediate immune cell recruitment to inflammatory sites.

Engagement of chemokine receptors with their ligands enhances cell adhesion by remodeling the actin cytoskeleton and activation of integrins. The importance of chemokine receptors in immunity is demonstrated by a rare genetic disease, WHIM syndrome, which is caused by gain-of-function mutations in CXCR4 and results in profound immune dysregulation.

One of the challenges with T cell therapy is to target and home the therapeutic cells to the area of interest, thus homing to the tumor and metastasis. Most T cells given as an intravenous transfusion end up in the lung, whereas metastases are frequently located in extra pulmonary tissue, thus, there is a need to guide and direct therapeutic cells to the area of interaction with tumor cells for T cell therapy to be effective. Herein is introduced sequences encoding chemokine receptors on a synthetic chromosome platform to provide expression of chemokine receptors on transfected T cells in order to provide homing guidance to the T cells. Expression of chemokine receptors on T cell direct these cells to the tumor and increase their overall therapeutic efficacy.

Chemokine ligands and receptors are not only expressed on immune cells. Tumors utilize chemokine receptor to direct them to other tissues which support their growth. Solid tumors often recruit inhibitory immune cells, such as T regulatory cells (Tregs) or Myeloid-Derived Suppressor Cells (MDSC), with their chemokine ligands which dampen anti-tumor immune responses. In this manner, tumors hijack important tolerance-inducing mechanisms to form an anti-inflammatory microenvironment, which facilitates tumor immune evasion.

Regulatory T cells (Treg) express the chemokine receptor CCR4 on the cells surface. CCR4 is attracted by the chemokines CCL17 and CCL22, and it is the main way of recruiting Tregs as part of regulating immune responses. However, tumor cells frequently develop the capacity to express CCL22 in their local environment as a part of their immune escape to avoid recognition and killing by tumor specific T cells. This means that cancer cells recruit Tregs to regulate the immune response of the tumor to promote tumor survival. With the expression of CCR4 on anti-tumor cells from a synthetic chromosome, anti-tumor T cells thereby home to tumor cells expressing CCL22 (and/or CCL17). In this manner the use of a synthetic chromosome allowing for expression of CCR4 in anti-tumor T cells provides a means for targeting and killing tumor cells. To utilize this observation the present invention provides embodiments wherein CCR4 is constitutively expressed using synthetic chromosomes. Accordingly, a constitutive expression of CCR4 allows recruitment of transfected effector T cells by a tumor induced Treg mechanism leading to initiation of tissue-/organ-specific homing of the transfected T effector cells. Combining such a constitutive expression of CCR4 e.g., with inducible expression of e.g., CCR6 is advantageous in cases where homing to cancer cells in the liver is desired (the use of the homing receptor CCR6 leads the anti-tumor cells to the liver and the use of the homing receptor CCR4 enables the cancer cells to recruit the anti-tumor cells).

### CCR4

CCR4 (SEQ ID NO:1) is the receptor for chemokines CCL17/Thymus- and Activation-Regulated Chemokine (TARC) and CCL22/Monocyte Derived Chemokine (MDC). CCR4 is the main chemokine receptor for Treg recruitment in the periphery. The binding of CCL22 to CCR4 induces activation and signaling by G proteins initiating actin shuffling and migration of the cell towards the chemokine gradient.

CCR4 is mainly expressed by T cells, and it is the receptor for two CC chemokine ligands, CCL17 and CCL22. Importantly, CCR4 expression is restricted to certain T cell subsets, such as T regulatory cells (Tregs), Th2, and Th17 cells. Mogamulizumab, a monoclonal antibody which is used in T cell leukemia/lymphoma (ATLL) and cutaneous T cell lymphomas (CTCLs) immunotherapy, specifically targets CCR4+ cells. The same antibody has been also successfully applied in other solid tumors because it depletes Tregs and disrupts the suppressive tumor microenvironment. This highlights the importance of CCR4 in Treg biology and provide an effective mean to interfere with Treg mediated immunosuppression in cancer patients. Combining CCR4 expression with CAR-T cells have been already proposed in CCL17+ Hodgkin lymphoma. By inclusion of CCR4 in synthetic chromosomes it is possible to drive a large number of leukocytes carrying the synthetic chromosome to the tumor microenvironment where otherwise immunosuppressant CCR4+ Tregs would dominate.

### CCR6

CCR6 (SEQ ID NO: 2) is the receptor for chemokine CCL20 or liver activation regulated chemokine (LARC) or Macrophage Inflammatory Protein-3 (MIP3A). Binding of CCL20 induces activation of CCR6 and G protein-initiated actin shuffling and migration of the cell towards the chemokine gradient.

CCR6 is a G-protein coupled receptor naturally expressed in lymphatic cells. It is unique with respect to the fact that this receptor binds solely to one ligand, Chemokine (C-C motif) ligand 20 (CCL20). The CCL20-CCR6 axis is involved in tissue inflammation and homeostasis but this natural axis is often hijacked in cancer progression. The liver is a common site for metastases from many cancer types, most commonly colorectal cancer. Colorectal cancer cells express both CCL20 and CCR6. This autocrine and paracrine loop leads to increased proliferation and migration of the cancer cells. Increased CCR6 expression in colorectal tumors is strongly associated with metastasis and poor prognosis for the patient. Animal studies where CCR6 is over expressed in CAR-T cells show that the cells have an increased migration to the tumor site and also infiltrate and clear the tumor when reaching the site. By inclusion of CCR6 in a synthetic chromosome it is possible to turn the tumor's weapons against it. It will help the T cells to migrate towards the tumor site and help to infiltrate the tumor.

### CXCR4

CXCR4 (SEQ ID NO: 3) is expressed in non-small cell lung cancer (NSCLC), prostate cancer, and breast cancer, which commonly metastasize into organs with constitutive CXCL12 (CXCR4 ligand) expression including the liver, bone marrow, skeletal muscle, kidney, and brain.

### CCR7

CCR7 (SEQ ID NO: 4) binds the chemokines CCL19 and CCL21, which are important for tissue homeostasis, immune surveillance, and tumorigenesis. CCR7 receptors have been shown to induce the pathobiology of breast cancer due to their ability to induce cellular proliferation and migration upon the binding of the cognate chemokine receptors.

### CXCR3

CXCR3 (SEQ ID NO: 5) has been reported to have a metastasis-promoting function in breast cancer, colon cancer, and osteosarcoma, as well as lung cancer. Several colon cancer cell lines constitutively express CXCR3 and the expression in the primary tumor lesion significantly correlates with lymphatic invasion and lymph node metastasis.

### CX3CR1

The CX3CR1 (SEQ ID NO: 6) is involved in the proliferation, survival and metastasis of various malignant tumor types, including clear cell renal cell carcinoma, prostate cancer, breast cancer, gastric cancer, pancreatic ductal adenocarcinoma and glioma tumors.

The involvement of CCR4, CCR6, CXCR4, CCR7, CXCR3 and CX3CR1 in forming metastases makes them suitable candidates for inducible expression on synthetic chromosome (such as hSynC) (Table 1).

TABLE 1
Examples of chemokine receptor genes that may be used
as inserts on synthetic chromosomes such as hSynC.

| Chemokine | | | |
|---|---|---|---|
| receptor | Ligand | Expression | Uniprot ID |
| CCR4 | CCL17, CCL22 | CCR4 is expressed on | P51679 (1996 Oct. 1 v1) |
| | | T regulatory cells | |
| CCR6 | CCL20 | CCR6 is upregulated | P51684(1998 Jul. 15 v2) |
| | | on metastatic CRC | |
| CXCR4 | CXCL12 | CXCR4 is upregulated | P61073-1 (2004 Apr. 26 v1) |
| | | on metastatic BC and | |
| | | NSLC | |
| CCR7 | CCL19, CCL21 | CCR7 is upregulated | P32248 (1996 Feb. 1 v2) |
| | | on metastatic BC, | |
| | | NSLC, CRC | |
| CXCR3 | CXCL9, | Expressed on CRC and | P49682 (1997 Nov. 1 v2) |
| | CXCL10 | promote lymph node | |
| | | metastases | |
| CX3CR1 | CX3CL1 | BC bone marrow | P49238 (1996 Feb. 1 v1) |
| | | metastasis | |

As shown in examples 6 and 7 herein, CCR4 encoded on a synthetic chromosome is expressed in a synthetic chromosome transfected tumor-recognizing T cell under the control of a weak constitutive promoter, such as PKG or EF1α, and gives an advantage in migrating the T cell to Treg infiltrated/diseased tissues, such as the immune-suppressed tumor microenvironment. Careful selection of promotor facilitates a desired expression level of CCR4 to ensure that the synthetic chromosome manipulated T cells reach the desired target such as e.g., the immune-suppressed tumor microenvironment. As an example, weak promotors ensure that high CCR4 expression does not cause marginalization of the synthetic chromosome manipulated T cells to irrelevant tissues, which would render them ineffective. In analogy, strong promotors may cause marginalization of the synthetic chromosome manipulated T cells to irrelevant tissues, which would render them ineffective.

In the examples 6 and 7 it is also shown that it is possible to combine CCR4 with other chemokine receptors, for example CCR6 or CXCR4. These latter receptors draw T cell migration to tumor metastases and will be under the control of exogenous regulation, such as an inducible promoter. If the synthetic chromosome manipulated T cell is used in subjects having developed metastases or being at risk of developing tumor metastasis, the induced expression of these receptors will reroute the anti-tumor T cells to the metastasis.

The construction and the structure of the synthetic chromosome are described in the following paragraphs.

The synthetic chromosome (Sync) is a small chromosome that is handled as a normal chromosome during cell division (mitosis) i.e., when the cell is preparing to divide it will also duplicate the Sync. In the sam manner as the odd number small Y chromosome the Sync will be copied and propagated intact in each cell division. When the Sync has been tested in mice it has been propagated intact for 4 generations of mice, meaning that the Sync is handled as an intact chromosome which does not integrate into host cell chromosomes and is stable for a life time. In cell lines we have demonstrated >60 generations of stable intact Sync propagation without integration.

Since the Sync is a non-integrating platform carrying large amount of genetic material, there is no risk that genetic material is integrated in host cell chromosome disrupting normal control of cell division leading to malignant transformation and cancer. This is in great contrast when viral vectors or CRISPR is used where there is a high risk of insertion of genetic material in open chromatin responsible for regulation of cell division.

Incorporation of One or More Chemokine Receptors Factors into Synthetic Chromosome

### Synthetic Chromosomes

To date, the genesis and development of mammalian artificial/synthetic chromosomes has relied on four principle means including:

“Top-down” approach: sequential truncation of pre-existing chromosomes arms to essential functional chromosome components including a centromere, telomeres, drug selectable marker, and DNA replication origins. As such, “top-down” artificial chromosomes are constructed to be devoid of naturally occurring expressed genes and engineered to contain DNA sequences(s) that permit site-specific integration of target DNA sequences onto the truncated chromosome (mediated via site-specific DNA integrates).

“Bottom-up” approach: co-introduction by cell transfection of chromosomal functional elements including DNA sequences associated with centromere function (e.g. large repeated arrays of human alpha-satellite sequences), telomeric sequences, and a drug selectable marker aiming for functional de novo assembly of the chromosomal components. The “bottom-up” also incorporates DNA sequences(s) that permit site-specific integration of target DNA sequences onto e.g. a truncated chromosome (mediated via site-specific DNA integrates).

Engineering of naturally occurring mini chromosomes: telomere-associated truncation of a marker chromosome containing a functional human neocentromere (possessing centromere function yet lacking alpha-satellite DNA sequences) and engineered to be devoid of non-essential DNA. As in the other approaches, these generated chromosomes can be engineered to contain DNA sequences(s) that permit site-specific integration of target DNA sequences.

“SATAC” approach: induced de novo chromosome generation by targeted amplification of specific chromosomal segments. In this methodology, large-scale amplification of pericentric/ribosomal DNA regions situated on acrocentric chromosomes are initially triggered by co-transfection of excess rDNA along with DNA sequences that allow for site-specific integration of target DNA sequences along with a drug selectable marker into pericentric regions of acrocentric chromosomes. During this process, targeting to the pericentric regions of acrocentric chromosomes with co-transfected DNA induces large-scale chromosomal DNA amplification, duplication/activations of centromere sequences, and subsequent breakage and resolution of the dicentric chromosome thereby resulting in a “break-off” satellite DNA-based synthetic chromosome containing multiple site-specific integration sites (termed platform chromosome).

Marker gene or genes used for cell identification and potential sorting could be applied to any available synthetic chromosome or could be integrated onto an endogenous chromosome. In the examples described herein, the human synthetic chromosome, hSync, is generated from human acrocentric chromosome 15 and contains multiple copies of a single recombination acceptor site (bacteriophage lambda attP), human ribosomal DNA, array(s) of LacO repeat sequences and at least one selectable marker gene.

Bioengineering of a synthetic chromosome requires the ability to target nucleic acid sequences of interest onto the synthetic chromosome and is typically accomplished by incorporating site-specific recombination sites onto the synthetic chromosome. Recombination systems that have been employed for these purposes include, but are not limited to: bacteriophage lambda integrase, Bacteriophage phiC31; Saccharomyces cerevisiae FLP/frt etc.

The strategy used to generate our human synthetic chromosome, hSync, is outlined in FIG. 1. In brief, an EF1αattPPuro cassette (SPB0125) containing an EF1a promoter, a 282 bp lambda-derived attP sequence, an array of 48 LacO repeats and the gene conferring puromycin resistance is co-transfected with an excess of a linearized human rDNA-containing vector (SPB0107) into the human HT1080 fibrosarcoma cell line. The rDNA facilitates integration of both vectors near the pericentric region of human acrocentric chromosomes and initiates synthetic chromosome formation. Importantly, the pEF1αattPPuro vector has been engineered to eliminate CpG sequences in order to diminish the potential host immune response that can be generated towards unmethylated CpG motifs as well as alleviate potential gene silencing of the drug resistance marker. Following integration of the SPB0125 vector into the pericentric region of a human acrocentric chromosome, the region undergoes amplification across the centromere thereby creating a dicentric chromosome. Upon resolution of the dicentric, a satellite artificial chromosome (SATAC) containing rDNA and the SPB0125 is created. The human Synthetic Chromosome developed from HT1080 cells is called hSync. Due to the amplification event, multiple attP sites are dispersed along the newly formed hSync, each of which is available for downstream bioengineering of the newly formed hSync. Drug resistant clones were evaluated by PCR targeting SPB0125 and SPB0107 sequences a candidate clone, HG3-4, was selected for subsequent analysis and evaluation. Presence of the synthetic chromosome was assessed by fluorescent in situ hybridization (FISH) directed towards pEF1αattPPuro or lacO sequences, centromeric and telomeric sequences. Single cell cloning and expansion of two independent clones, HG3-4ssc3F and HG3-4ssc4D, demonstrated hSync mitotic stability over approximately 50 population doublings in the HT1080 cell line. The hSync was then transferred into Chinese Hamster Ovary CHO-K1 cells, which constitutes the cell line of our choice for future bulk production of chromosomes. FISH and PCR were used to confirm the chromosomal integrity and the presence of human specific alpha satellite sequences and the SPB0125 attP sequences (FIG. 2).

The hSync can be further bioengineered to contain one or more marker genes for use in cell identification and purification by unidirectional insertion of each marker using a lambda integrase protein that functions independently of the native helper proteins (e.g., IHF, Xis). In addition, the hSync, once bioengineered with the marker gene or genes of choice, can be isolated and transferred to a recipient cell line of interest while retaining all bioengineered and native structural elements and stably maintained in the recipient cell line for well over 50 population doublings.

### Structures of Synthetic Chromosomes

At the most basic level a chromosome can be functionally defined as having centromeres for faithful segregation to daughter cells at each cell division; telomeres for protection of the ends of the nucleic acid molecule; and origins of replication for carefully and precisely copying the chromosome (two copies for mitosis and four copies for meiosis) prior to each cell division.

Structural elements of engineered synthetic chromosomes can include, but are not limited to, multiple rDNA, functional centromeric sequences and/or telomeric sequences; multiple bacteriophage lambda-derived attP (or other) sites (for targeted integration and loading of nucleic acid cassettes via delivery vectors); an array of multiple lacO repeats (for selection or isolation of chromosome-bearing cells using flow sorting; as well as selectable markers and/or tags (e.g., nucleic acid sequences encoding drug resistance), nucleic acid sequences encoding reporter proteins fused to fluorescent or other tags (for tracking and/or visualizing the engineered synthetic chromosome(s) using microscopy). or nucleic acid binding sites for tagged proteins,

Markers can be used to positively or negatively select and/or isolate living cells. Tags can be used to visualize synthetic chromosomes, in some cases within chromosome-bearing cells. Markers, and reporter genes can include one or more detectable signals, such as, for example, fluorescent, luminescent or phosphorescent tags (which can emit signals at various distinct wavelengths on the visible spectrum allowing “chromosome painting” and visualization of engineered synthetic chromosomes, or other detectable signals). Markers and/or tags may also allow isolation of cells carrying the synthetic chromosome(s), via flow sorting or by isolation using magnetic beads.

Fluorescent proteins of particular use include but are not limited to TagBFP, TagCFP, TagGFP2, TagYFP, TagRFP, FusionRed, mKate2, TurboGFP, TurboYFP, TurboRFP, TurboFP602, TurboFP635, or TurboFP650 (all available from Evrogen, Moscow); AmCyan1, AcvGFP1, ZsGreen1, ZsYellow1, mBanana, mOrange, mOrange2, DsRed-Express2, DsRed-Express, tdTomato, DsRed-Monomer, DsRed2, AsRed2, mStrawberry, mCherry, HcRed1, mRaspberry, E2-Crimson, mPlum, Dendra 2, Timer, and PAmCherry (all available from Clontech, Palo Alto, CA); HALO-tags; infrared (far red shifted) tags (available from Promega, Madison, WI); and other fluorescent tags known in the art, as well as fluorescent tags subsequently discovered. For example, in some embodiments, SNAP-tags may be used to identify transfected cells following transfection.

As a synthetic chromosome is autonomous and non-integrating, replicating and segregating 1:1 with cells produced by each cell division; it has the capacity to carry megabases of inserted DNA (as needed for multiple promoters, which may be linked to the same or a different visually observable fluorescent or luminescent marker). Using these synthetic chromosomes, single cells can be tracked within a population of cells/tissue/organism, and differentiation states and responses to environmental cues can be observed at single cell resolution.

Previous art requires pre-engineering of the cell line to be used, involving integration of recombination sites into the endogenous chromosomes; this must be done for each cell type being tested. Hence, the exact location of the responsive elements may not be the same from cell to cell tested. With a synthetic chromosome, the responsive elements are all contained on the chromosome and moved to the cell type to be tested collectively in the same chromosomal context allowing direct comparison between different cell types with the same reporter readout construct (i.e., synthetic chromosome).

Silencing and/or variable expression of therapeutic genes introduced using cellular and/or gene therapy is a major hurdle to achieving consistent and stable therapeutic efficacy. Insulators, first identified in the 1990s, are genetic elements that establish high-level chromatin architecture and protect promoters from the adjacent chromatin environment. These elements contain binding sites for proteins that promote changes to chromatin structure that define domains of transcriptional activity. Insulators come in two distinct types based on how they protect promoters, barrier insulators and enhancer-blocking insulators. Barrier insulators prevent spreading of closed and transcriptionally inactive chromatin, e.g., heterochromatin, from bordering regions thereby preventing gene silencing and ensuring open chromatin structure with continued gene expression. This activity requires two barrier insulators, one on each side of the region to be protected. Enhancer-blocking insulators prevent undesirable expression by blocking the action of an enhancer if an integrated promoter is placed near to it. Although fewer than 100 insulator elements have been characterized, data suggest there are likely thousands of these cis-acting sequences that can function as either cell type-specific or cell type-independent insulators.

Alternative genetic elements, called ubiquitous chromatin-opening elements (UCOE), that are responsible for establishing a transcriptionally competent open chromatin structure at ubiquitously expressed housekeeping genes have been described. In contrast to insulators, these elements are positioned directly upstream of the promoter driving expression of the gene of interest and function to maintain the chromatin in an open configuration so that transcription factors and RNA polymerases can gain access. Very few UCOEs have been characterized to date but their efficacy on adjacent gene expression can vary depending on orientation of the UCOE, promoter, and cell type.

In order to amplify the amount of the synthetic chromosome a first transfection may be carried out into a producer cell line such as CHO or a human cell line such as HT1080.

### Chromosome Isolation—General Description

Manufacturing cells carrying the chromosome are arrested in metaphase of mitosis with chromosomes condensed by addition of an agent that arrests cells in metaphase (e.g., KaryoMAX™) to the cell culture medium. The following day cells are harvested, lysed, the condensed chromosomes are isolated, filtered and labeled. The chromosomes are then applied to a flow cytometer and the synthetic chromosome is flow sort purified from the endogenous chromosomes using chromosome size and the applied label or labels as sorting parameters. The purified chromosomes are washed and used in downstream applications.

### Transfection Methods

### Lipid Mediated Transfection

During chromosome manufacturing, mitotically active cells are transfected with standard lipid-based transfection reagents following the manufacturers recommended conditions for the specific transfection agent. For each cell line, transfection conditions (e.g., lipid:DNA ratio) are optimized. Constructs to be loaded onto the chromosome are co-transfected with an engineered bacteriophage lambda mutant integrase that drives unidirectional recombination in mammalian cells. Twenty-four hours post-transfection the cells are placed on drug selection.

### Mechanical Transfection

Various methods utilizing mechanical transfection have been described in the literature. Common for them all is that the cell membrane is destabilized using mechanical force. The mechanical force can originate from a variety of forces (e.g., cell squeezing). Examples of mechanical transfection include mechanoporation and hydroporation, As a result, pores in the cell membrane are created by cellular physical contact with a solid substrate (mechanoporaton) or from shear forces generated from the surrounding fluid (hydroporation) thereby permitting entry into the cell of the transfecting material.

### Transfection Via Injection

Injecting a chromosome directly into the nucleus of a cell is highly effective but very time and labor intense. In this method, transferring genetic material into the cell is accomplished by using glass micropipettes or metal microinjection needles into the cell nucleus.

Vector Transfection with Electroporation

Vectors carrying the manipulated gene, or a wild-type control is transfected into cell lines or primary cells using electroporation. In electroporation the cells are mixed with the vector and a transfection reagent and then run through an electric field. The electric field will transiently destabilize the cellular membrane allowing for the vector to pass through into the cell. For each cell type transfection reagents and electroporation program is optimized. The transient expression is analyzed within 72 hours using flow cytometer or sorting or monitoring gene expression.

Transfer of engineered flow sort purified chromosomes to recipient cell lines is performed utilizing commercially available chemical transfection methods. However, T cells are small and their cytoplastic space has a limited capacity for the type of endocytosis needed in chemical transfections. A range of chemical and mechanical transfection methods can be used and may be adapted for delivery into T cells or other cells with limited capacity for endocytosis.

Genomic manipulation of markers and vector experiments—general description

Using the genome browsers from Ensembl, NCBI, and UCSC the cDNA sequence of the gene of interest is identified and investigated for functional domains. The functional domains of the protein are annotated within the gene sequence and multiple manipulated versions of the gene of interest may be designed and their synthesis ordered from a commercial vendor. After determining the optimal version of the gene of interest as expressed from a plasmid vector in the cell line of interest, the chosen gene of interest is bioengineered onto the synthetic chromosome. Once confirmed by quality control, the bioengineered chromosome carrying the manipulated gene of interest is then transferred to the manufacturing cell line.

### Selection and Regulation of Expression of Chemokine Receptors

The current invention equips leukocytes or other cells with chemokine receptors allowing them to traffic to diseased tissue such as inflammation, a primary tumor or metastases. Upon homing the cells may exert their function and combat the disease. Selection of appropriate chemokine receptors is a key step and importantly a synthetic chromosome such as, e.g., hSync allows us to use more than one chemokine receptor if needed. One prominent use of the present invention is to constitutively express a chemokine receptor that targets tumor tissue, such as CCR4 that facilitates a regulatory T cell like movement to the tumor. Alternatively, a receptor targeting a specific organ where the cancer occurs may also be used (Table 1). This can then be combined with an inducible chemokine receptor that allows for homing to metastatic sites (Table 1). Thus, it is possible to alter the homing pattern from the primary tumor to metastases as the cancer progresses.

The chemokine receptor coding genes are designed to be under the regulation of promoters such as viral promoters (CMV, SV40 or other), truncated or full size eukaryotic promoters (PKG, EF-1a), truncated or full size promoters of T cells (interleukin-2 or other), or inducible promoters (TET or other). Constitutive promoters (such as PKG, EF-1a) will be combined with inducible promoter (such as TET) to regulate the expression of distinct chemokine receptors.

Silencing and/or variable expression of therapeutic genes introduced using cellular and/or gene therapy is a major hurdle to achieving consistent and stable therapeutic efficacy. Insulators, first identified in the 1990s, are genetic elements that protect promoters from the adjacent chromatin environment. These elements contain binding sites for proteins that promote changes to chromatin structure that define domains of transcriptional activity. Insulators come in two distinct types based on how they protect promoters, barrier insulators and enhancer-blocking insulators. Barrier insulators prevent spreading of closed and transcriptionally inactive chromatin, e.g., heterochromatin, from bordering regions thereby preventing gene silencing and ensuring open chromatin structure with continued gene expression. This activity requires two barrier insulators, one on each side of the region to be protected. Enhancer-blocking insulators prevent undesirable expression by blocking the action of an enhancer if an integrated promoter is placed near to it. Although fewer than 100 insulator elements have been characterized, data suggest there are likely thousands of these cis-acting sequences that can function as either cell type-specific or cell type-independent insulators.

Alternative genetic elements, called ubiquitous chromatin-opening elements (UCOE), that are responsible for establishing a transcriptionally competent open chromatin structure at ubiquitously expressed housekeeping genes have been described. In contrast to insulators, these elements are positioned directly upstream of the promoter driving expression of the gene of interest and function to maintain the chromatin in an open configuration so that transcription factors and RNA polymerases can gain access. Very few UCOEs have been characterized to date but their efficacy on adjacent gene expression can vary depending on orientation of the UCOE, promoter, and cell type.

### Cells for Use in the Invention

The cells that are to be transfected with a synthetic chromosome (e.g. hSync) should be to promote that the cells reached the target tissue (e.g. beta cells to be directed to the liver, or in rare disease to direct the cells to the spleen etc.), or to produce tumor immunity. When they are directed to the appropriate tumor or metastasis site they will act, directly or indirectly in a tumoricidal manner. Specifically, the cells will be syngeneic leukocytes purified from the blood, the tumor draining lymph node or from tumor infiltrating lymphocytes from the patients. Their action may be cytotoxic, proinflammatory and by inhibiting immunosuppressive agents withing the tumor.

The aim of transfecting cells with a synthetic chromosome (e.g., hSync) is to take advantage of the high load capacity of the chromosomes to carry genes of interest. In this case sequences encoding chemokine receptors are loaded on the chromosomes so—when the chromosomes are contained in cells—the cells should reach the target tissue and there express the chemokine receptor(s) to obtain increased proliferation and/or activation of the cells in question in an autocrine or paracrine manner. If it is paracrine signaling, then the cells being proliferated and/or activated may be different from those carrying the chromosomes.

The cells in question may be leukocytes, tumor infiltrating cells, lymphocytes such as T cells, B cells, NK cells or the like.

In the case where the cells are directed to a tumor or metastasis site they will act, directly or indirectly in a tumoricidal manner. Specifically, the cells will be syngeneic leukocytes purified from the blood, the tumor draining lymph node or from tumor infiltrating lymphocytes from the patients. Their action may be cytotoxic, proinflammatory and/or by inhibiting immunosuppressive agents withing the tumor.

### Expansion of Cells Containing Chromosome(s) Encoding Chemokine Receptors

Expansion of cells containing chromosomes are according to normal cell culturing methods for the cell type used.

### Use of Cells Containing Chromosome(s) Encoding Chemokine Receptors

Chemokine receptors are chemokine inducible initiations of migration towards the chemokine gradient allowing site specific recruitment of cells. For example, CCR9 expressing cells are recruited by the chemokine CCL25 resulting in gut homing. Introducing chemokine receptors onto the hSync will allow directed homing of cells. The directing homing can be used to increase metastases homing of effector cells, direct stem cells to the site for terminal differentiation or recruit genetically modified cells to the site of preferred action. In colorectal cancer tumor cells develop to express CCR6 homing the metastatic cells to the CCL20 expressing liver. Thus, the use of CCR6 on the hSync will preferentially attract synthetic chromosome-bearing cells (denoted Cromo-T cells) to the liver in order to identify and eliminate metastases. Thus, different chemokine receptors will be able to direct effector cells to the site of metastases by preferential expression of lung, skeleton, brain homing chemokine receptors. Many solid tumors attract regulatory T cells (Tregs), T cells that inhibit the immune function of effector T cells thus promoting survival of the tumor. Tregs express CCR4 on the cells surface and tumors that have developed the local expression CCL17 or CCL22 will attract Tregs as an immunescape mechanism. To use Cromo-T cells equipped with CCR4 expression will therefore attract T effector cells to the tumor and induce tumor recognition and elimination.

The use of chemokine directed homing can be used to target genetically modified b cells to treat diabetes to for example the liver. Correction of metabolic disorders where homing to the liver is warranted is another example. Stem cells and iPSC can be targeted to end up at a useful site. For example, expression of CXCR4 will promote homing to the skeleton and useful for osteoporosis.

### Formulation of Pharmaceutical Compositions

### Qualitative and Quantitative Composition

T cells from blood, sentinel node or from the tumor (TIL) will be transfected with the synthethic chromosome resulting in chromosome-bearing cells (also denoted Cromo T cells). For release we expect more than 90% CD4+ and CD8+ T cells in the transfusion. In addition to sterility and absence of endotoxins, the majority of cells will respond after antigen specific stimulus with IL-2 and or IFN-g response measured by ELISA or intracellular FACS. The dosage of the final product remains to be established but in a previous study we administered autologous tumor reactive sentinel node derived T-cells at a median dose of 153×10⁶ cells per patient without any treatment related toxicity. The lowest dose where we have found a partial response is 50×10⁶ cells. We found a dose response where patients having received above 100×10⁶ cells had a higher chance of responding with complete response. These cells were not carrying a synthetic chromosome, thus no genetically enhanced tumor response was evaluated. We expect that the introduction of the synthetic chromosomes with cytokines and/or homing elements will allow for a lower effective dose, which will be determined in clinical studies. Consequently, the dose of cells will likely range from 10⁶-10⁸ viable T cells, similar to the dose range used in Chimeric antigen receptor T-cell therapies.

### Pharmaceutical Form

The composition is in the form of a cell suspension for infusion. The transfected patient T-cells are harvested, washed with isotonic saline solution and then resuspended in isotonic saline solution supplemented with 1% human serum albumin.

### Administration

The cells carrying the synthetic chromosome will generally be used in an amount effective to treat, ameliorate, reduce the symptoms of, or prevent additional symptoms of a particular disease being treated. A composition comprising the cells (carrying the chromosome) may be administered therapeutically to achieve therapeutic benefit or prophylactically to achieve prophylactic benefit. By therapeutic benefit is meant eradication or amelioration of the underlying disorder being treated, e.g., eradication or amelioration of the underlying hyperproliferative disorder such as cancer, autoinflammatory disease or allergy, or autoimmune disease, for example, and/or eradication or amelioration of one or more of the symptoms associated with the underlying disorder such that the patient reports an improvement in feeling or condition, notwithstanding that the patient may still be afflicted with the underlying disorder. (For example, administration of the composition to a patient suffering from an allergy provides therapeutic benefit not only when the underlying allergic response is eradicated or ameliorated, but also when the patient reports a decrease in the severity or duration of the symptoms associated with the allergy following exposure to the allergen.) Therapeutic benefit also includes halting or slowing the progression of the disease being treated, regardless of whether improvement is realized.

For prophylactic administration, the composition may be administered to a patient at risk of developing a cancer, such as a subject who is determined to be genetically predisposed to developing a particular cancer, such as a subject having a family history of particular cancers, or a subject who has undergone genetic testing and found to have such predisposition. In another example, if it is unknown whether a patient is allergic to a particular drug, the therapeutic composition may be administered prior to administration of the drug to avoid or ameliorate an allergic response to the drug. Alternatively, prophylactic administration may be applied to avoid the onset of symptoms in a patient diagnosed with the underlying disorder. The composition may also be administered prophylactically to a currently asymptomatic individual who is repeatedly exposed to one or more agents known to provoke disease onset, in order to delay or prevent the onset of the disease or disease symptoms. The amount of therapeutic composition administered will depend upon a variety of factors, including, for example, the particular indication being treated, the mode of administration, whether the desired benefit is prophylactic or therapeutic, the severity of the indication being treated and the age and weight of the patient, etc.

The compositions disclosed herein may be administered through any mode of administration. These compositions may be administered by injection, for example, intravenously, subcutaneously, intramuscularly, or may be administered intranasally, intraperitoneally, intracranially or intrathecally, by inhalation, orally, sublingually, by buccal administration, topically, transdermally, or transmucosally. In some aspects, the compositions are injected intravenously. In some embodiments, the compositions may be administered enterally or parenterally. In some embodiments, compositions are administered by subcutaneous injection, orally, intranasally, by inhalation, or intravenously.

The term “unit dosage form,” as used herein, refers to physically discrete units suitable as unitary dosages for human and animal subjects, each unit containing a predetermined quantity of compounds/therapeutic agents of the present disclosure calculated in an amount sufficient to produce the desired effect in association with a pharmaceutically acceptable diluent, carrier or vehicle.

As used herein, the phrase “pharmaceutically acceptable carrier” refers to a carrier medium that does not interfere with the effectiveness of the biological activity of the active ingredient. Such a carrier medium is essentially chemically inert and nontoxic.

As used herein, the phrase “pharmaceutically acceptable” means approved by a regulatory agency of the Federal government or a state government, or listed in the U.S. Pharmacopeia or other generally recognized pharmacopeia for use in animals, and more particularly for use in humans.

As used herein, the term “carrier” refers to a diluent, adjuvant, excipient, or vehicle with which the therapeutic is administered. Such carriers can be sterile liquids, such as saline solutions in water, or oils, including those of petroleum, animal, vegetable or synthetic origin, such as peanut oil, soybean oil, mineral oil, sesame oil and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The carrier, if desired, can also contain minor amounts of wetting or emulsifying agents, or pH buffering agents. Pharmaceutical compositions can take the form of solutions, suspensions, emulsion, tablets, pills, capsules, powders, sustained-release formulations and the like. The composition also can be formulated as a suppository, with traditional binders and carriers such as triglycerides. Examples of suitable pharmaceutical carriers are described in Remington's Pharmaceutical Sciences by E. W. Martin. Examples of suitable pharmaceutical carriers are a variety of cationic polyamines and lipids, including, but not limited to N-(1(2,3-dioleyloxy)propyl)-N,N,N-trimethylammonium chloride (DOTMA) and diolesylphosphotidylethanolamine (DOPE). Liposomes may be suitable carriers for uses of the present disclosure. The compositions may include a therapeutically effective amount of additional compounds, with or without a suitable amount of carrier so as to provide the form for proper administration to the subject. The formulation should suit the mode of administration.

### Other Aspects of the Invention

Combination with Other Genes—Sorting, Identification, Etc.

Based on the long clinical history in treating polygenic disorders such as cancer, favorable clinical outcomes are often obtained utilizing a multi-targeted approach as compared to single therapeutic administration. Likewise, precision medicine approaches incorporating cell and gene therapy approaches will be enhanced by the delivery of multiple gene products targeting multiple genetic networks that are altered in the tumor cell environment.

For the delivery of multigene components as part of a cell and gene therapy regimen, incorporation of multiple gene products harbored on a single synthetic chromosome offers a significant advantage over the integration of multiple gene products dispersed across the host genome or incorporation into a single site in the host genome. Integration of therapeutic gene products into the host genome runs the risk of insertional mutagenesis leading to altered cell physiology and potential immortalization. Targeting “safe harbors” in the genome can result in altered gene expression of neighbor gene loci. In addition, random targeting of genes into the genome can lead to rapid gene silencing of the therapeutic product due to integration in a genomic environment refractory to robust gene expression.

The incorporation of multiple gene therapeutic products onto a synthetic chromosome alleviates the potential problems associated with targeting the native host genome. A synthetic chromosome resides outside of the host chromosomes thereby avoiding potential insertional mutagenesis and/or integration into genome regions not permissive to robust gene expression. The incorporation of multiple therapeutic gene factors onto a synthetic chromosome ensures consistent segregation through multiple cell divisions, i.e. linkage disequilibrium. In contrast, the incorporation of multiple gene products dispersed throughout the genome increases the risk of mitotic malsegration of individual gene components. Bioengineering of a synthetic chromosome with multiple gene products permits the incorporation of multiple factors that can enhance robust, long-term therapeutic production with consistent product stoichiometry. Currently, the limited carrying capacity of gene transfer vectors seen in viral-mediated gene delivery does not allow substantial incorporation of factors that allow for long-term gene expression. Incorporation of multiple gene factors onto a synthetic chromosome permits rapid isolation and transfer of a bioengineered synthetic chromosome into multiple cell types, a process not allowed when the factors are dispersed crossed the native host genome.

As mentioned below it is possible to insert other genes of interest into a chromosome carrying genes for one or more chemokine receptors. Relevant genes could be:
i) genes encoding proteins for identifying and sorting the cells,
ii) genes encoding proteins for tracking the in vivo path of the cells after administration,
iii) genes encoding proteins acting as growth factors,
iv) genes encoding chimeric antigen receptors,
v) nucleic acid sequences encoding proteins or lncRNAs for safety switches that can i) induce cell death, and/or inactivate the function of the chromosome,
vi) genes and/or nucleic acid sequences for regulating expression of one or more proteins expressed by genes as described above.

Examples of genes encoding proteins for identifying and sorting or for tracking the in vivo faith of the cells after administration are e.g. CD34, trCD34, CD20, trCD20, CD19, trCD19, CD14, and trCD14.

Examples of cytokines acting as growth factors:

| Cytokine | Function | UniProt ID |
|---|---|---|
| IL-2 | Promotes the growth and development of T cells | P60568 |
| | | (1986 Jul. 21 v1) |
| IL-7 | Regulating the development and homeostasis of T cells | P13232-1 |
| | | (1990 Jan. 1 v1) |
| IL-12 | Promoting Th1 differentiation, induces cytotoxic | IL-12A: P29459 (2002 Jan. 23 v2) |
| | capacities of T and NK cells | IL-12B: P29460 (1993 Apr. 1 v1) |
| IL-15 | Enhancing the proliferation and survival of T cells | P40933-1 |
| | | (1995 Feb. 1 v1) |
| IL-18 | Stimulating production of IFN γ, activating NK cells and | Q14116-1 |
| | monocytes | (1996 Nov. 1 v1) |
| IL-21 | Enhances the proliferation and cytotoxic activity of T | Q9HBE4-1 |
| | cells | (2019 Apr. 10 v3) |
| IFN-a | Enhances proliferation and cytotoxicity of CD8 T cells | P01563 |
| | | (2022 May 25 v2) |

Examples of cytokine genes used as inserts on synthetic chromosomes such as e.g. hSync

Examples of genes encoding chimeric antigen receptors are e.g.

| TAA | Antibody source for CAR |
|---|---|
| CD276 | h8H9 |
| CEA(v1) | hC2-45 |
| Her 2 | H4D5 |
| GUCY2C | 5F9 |
| CEA(v2) | Labetuzumab |
| EGFR | Cetuximab |
| EpCAM | Edrecolomab |
| MUC1 | 10A |
| C-MET | Emibetuzumab |

Examples of nucleic acid sequences encoding proteins or RNAs for safety switches that can i) induce cell death, and/or inactivate the function of the chromosome are found in the following table:

| | | Accession | | |
|---|---|---|---|---|
| Gene name | Also known as | number | NCBI Gene ID | Function |
| BCL-2 | PPP1R50 | NG_009361.1 | 596 | Anti-apoptotic |
| BCL2L1 | BCL-XL | NG_029002 | 598 | Anti-apoptotic |
| BCL2L2 | BCL-W | NM_004050.5 | 599 | Anti-apoptotic |
| | | NM_001199839.2 | | |
| BCL-A1 | Bfl-1 | NG_029487 | 597 | Anti-apoptotic |
| MCL1 | BCL2L3, EAT, | NG_029146 | 4170 | Anti-apoptotic |
| | TM | | | |
| BAX | BCL2L4 | NG_012191 | 581 | Pro-apoptotic |
| | | | | pore-formers |
| BAK1 | CDN1, BCL2L7 | NM_001188.4 | 578 | Pro-apoptotic |
| | | | | pore-formers |
| BOK | BOKL, BCL2L9 | NG_029488 | 666 | Pro-apoptotic |
| | | | | pore-formers |
| BCL2L13 | BCL-rambo | NM_015367.4 | 23786 | Pro-apoptotic |
| | | | | pore-formers |
| BAD | BBC2, BCL2L8 | NM_004322.3 | 572 | Pro-apoptotic |
| | | NM_032989.3 | | BH3-only protein |
| BID | FP497 | NM_001196.4 | 637 | Pro-apoptotic |
| | | NM_197966.3 | | BH3-only protein |
| | | NM_197967.2 | | |
| BIK | BP4, NBK, BIP1 | NM_001197.5 | 638 | Pro-apoptotic |
| | | | | BH3-only protein |
| BCL2L11 | BIM, BAM, BOD | NG_029006 | 10018 | Pro-apoptotic |
| | | | | BH3-only protein |
| BMF | | NM_001003940.2 | 90427 | Pro-apoptotic |
| | | | | BH3-only protein |
| HRK | DP5, HARAKIRI | NM_003806.4 | 8739 | Pro-apoptotic |
| | | | | BH3-only protein |
| PMAIP1 | NOXA | NM_001382617.1 | 5366 | Pro-apoptotic |
| | | | | BH3-only protein |
| BBC3 | PUMA, JFY1 | NG_031991 | 27113 | Pro-apoptotic |
| | | | | BH3-only protein |

Examples of genes and/or nucleic acid sequences for regulating expression of one or more proteins expressed by genes as described above are inducible and/constitutive promoters.

In particular the chromosomes described herein may comprise nucleic acid sequence encoding for one of more safety switch. Therefore, the chromosomes according to the invention may in addition also contain nucleic acid sequences as described in the following.
1. A synthetic chromosome comprising a nucleic acid sequence encoding an inducible safety switch.
2. A synthetic chromosome according to item 1, wherein the safety switch when expressed induces cell death of a cell carrying the chromosome.
3. A synthetic chromosome according to item 2, wherein the cell death is due to apoptosis.
4. A synthetic chromosome according to item 3, wherein apoptosis is due to signaling in the intrinsic pathway.
5. A synthetic chromosome according any one of the preceding items, wherein expression of the safety switch is inducible.
6. A synthetic chromosome according to any one of items 1-3, 5, wherein the safety switch is one or more pro-apoptotic proteins.
7. A synthetic chromosome according to item 6, wherein the one or more pro-apoptotic proteins belongs to BCL-2 protein family or is a caspase.
8. A synthetic chromosome according to item 7, wherein the one or more pro-apoptotic proteins are selected from Table 1—Table of proteins in the BCL-2 family.
9. A synthetic chromosome according to item 8, wherein the BCL-2 protein is selected from BBC3, and BCL2L11.
10. A synthetic chromosome according to item 7, wherein the caspase is caspase-9.
11. A synthetic chromosome according to item 1, wherein the safety switch—when expressed—induces inactivation of the chromosome carried by the cell.
12. A synthetic chromosome according to item 11, wherein the safety switch comprises at least one Xic gene product selected from the group consisting of Xist and Tsix.
13. A synthetic chromosome according to any one of the preceding items, wherein the chromosome comprises a further nucleic acid sequence encoding for an anti-apoptotic protein.
14. A synthetic chromosome according to item 13, wherein the anti-apoptotic protein belongs to BCL-2 family.
15. A synthetic chromosome according to item 14, wherein the anti-apoptotic protein is selected from BCL-2, BCL2L1, BCL2L2, BCL-A1, and MCL1.
16. A cell comprising a synthetic chromosome as defined in any one of the preceding items.
17. A cell according to any of the preceding items for medical use, veterinary use, or diagnostics
18. A composition comprising a synthetic chromosome as defined in any one of items 1-15 and an additive.
19. A composition comprising a cell as defined in any one of items 16-17 and an additive.
20. A synthetic chromosome according to any one of items 1-15 comprising one or more nucleic acids encoding for one or more proteins selected from surface markers, growth factors, chemokine receptors, and chimeric antigen receptors.
21. A synthetic chromosome according to item 20, wherein the surface markers, growth factors, chemokine receptors, and chimeric antigen receptors are as described herein.

### Safety Switches

Because synthetic chromosomes are extraordinarily useful as carriers of large nucleic acid sequences, they can be designed to contain multiple regulatory sequences that can coordinately regulate expression of multiple genes from the chromosome. However, at certain times or in some situations, it may be important to turn off one or more genes introduced into cells via the synthetic chromosome, or to inactivate the entire chromosome. Such a safety switch or inactivation switch may be used if, for example, there is an adverse reaction to the expression of the gene product(s) from the synthetic chromosome requiring termination of treatment.

In one example of a safety switch, a whole-chromosome-inactivation switch may be used, such that expression of genes on the synthetic chromosome are inactivated but the chromosome-containing cells remain alive. Alternatively, a synthetic chromosome-bearing therapeutic cell-off switch could be used in a cell-based treatment wherein, if the synthetic chromosome is contained within a specific type of cell and the cells transform into an undesired cell type or migrate to an undesirable location and/or the expression of the factors on the synthetic chromosome is deleterious, the switch can be used to kill the cells containing the synthetic chromosome, specifically.

A safety switch may be engineered on the synthetic chromosome, or into the recipient cells, such that the safety switch is employed to shut off the synthetic chromosome, or genes encoded upon the synthetic chromosome, when they have served their purpose and are no longer needed. Thus, the entire synthetic chromosome introduced into cells can itself be inactivated (“chromosome OFF”), or some or all of the genes contained on the synthetic chromosome can be turned off (“genes OFF”). Further, one or more such safety switches can be used to regulate the activity of one or more genes encoded upon and/or expressed from the synthetic chromosome.

Alternatively, cells bearing a synthetic chromosome may need to be eliminated by inducing a cell to kill itself or to be killed in a cell death pathway. A cell-OFF safety switch can be included as a feature on the synthetic chromosome and may involve nucleic acid sequences encoding one or more proteins triggering a cell death pathway such as pro-apoptotic proteins or may make use of regulatory nucleic acids. Another method of providing a cell-OFF safety switch can involve engineering the recipient cells that will carry the synthetic chromosome to encode a system of apoptosis-inducing as well as counterbalancing anti-apoptotic proteins (or regulatory nucleic acids) such that the synthetic chromosome-bearing cells can be steered down an apoptotic pathway to eliminate these cells from a population.

Thus, the expression of genes encoded on the synthetic chromosome can be safely regulated and exquisitely coordinated through the use of one or more safety switches, wherein, for example, a first gene borne by the synthetic chromosome is turned on to produce a first gene product that negatively regulates expression of a second gene.

Apoptotic signaling pathways include (i) an extrinsic pathway, in which apoptosis is initiated at the cell surface by ligation of death receptors resulting in the activation of caspase-8 at the death inducing signaling complex (DISC) and, in some circumstances, cleavage of the BH3-only protein BID; and (ii) an intrinsic pathway, in which apoptosis is initiated at the mitochondria and is regulated by BCL2-proteins. Activation of the intrinsic pathway results in loss of mitochondrial membrane potential, release of cytochrome c, and activation of caspase-9 in the Apaf-1 containing apoptosome. Both pathways converge into the activation of the executioner caspases, (e.g., caspase-3). Caspases may be inhibited by the Inhibitor of apoptosis proteins (IAPs). The activities of various antiapoptotic BCL-2 proteins and their role in solid tumors is under active research, and several strategies have been developed to inhibit BCL2, BCL-XL, BCLw, and MCL1. Studies of several small molecule BCL-2 inhibitors (e.g., ABT-737, ABT-263, ABT-199, TW-37, sabutoclax, obatoclax, and MIM1) have demonstrated their potential to act as anticancer therapeutics. The BCL2-family includes: the multidomain pro-apoptotic proteins BAX and BAK mediating release of cytochrome c from mitochondria into cytosol. BAX and BAK are inhibited by the antiapoptotic BCL2-proteins (BCL2, BCL-XL, BCL-w, MCL1, and BCL2A1). BH3-only proteins (e.g., BIM, BID, PUMA, BAD, BMF, and NOXA) can neutralize the function of the antiapoptotic BCL2-proteins and may also directly activate BAX and BAK.

Bcl-2 proteins can be further characterized as having antiapoptotic or pro-apoptotic function, and the pro-apoptotic group is further divided into BH3-only proteins (‘activators’ and ‘sensitizers’) as well as non-BH3-only ‘executioners’. Enhanced expression and/or post-transcriptional modification empowers ‘activators’ (Bim, Puma, tBid and Bad) to induce a conformational change in ‘executioners’ (Bax and Bak) to polymerize on the surface of mitochondria, thereby creating holes in the outer membrane and allowing cytochrome c (cyto c) to escape from the intermembrane space. In the cytoplasm, cyto c initiates the formation of high-molecular-weight scaffolds to activate dormant caspases, which catalyze proteolytic intracellular disintegration. Destruction of the cell culminates in the formation of apoptotic bodies that are engulfed by macrophages. Antiapoptotic Bcl-2 proteins like Bcl-2, Mcl-1, Bcl-XL and A1, also known as ‘guardians’, interfere with the induction of apoptosis by binding and thereby neutralizing the pro-apoptotic members.

### Cell Death

Cells can die from many different reasons, they can die from an injury, from being killed by another cell, from starvation or via suicide. Excessive cell death can result in diseases like neuro degenerative diseases, while insufficient cell death may lead to cancers and tumor formation. Fortunately, non-accidental cell death is highly regulated at multiple levels. Cell death is divided into several categories, primarily based on the mode of initiation, but there is a substantial interplay between them. Most of the programs will be activated whence the point of no return has been reached.

### Killing

Cells can be killed by other cells; this is one function of the immune system. To kill intruding parasites, virus infected cells and cancer cells the immune system has many weapons in its arsenal. Both Natural Killer cells and Cytotoxic T-cells have cytotoxic granule packed with pore-forming perforin and apoptosis inducible Granzyme B. Polymerized perforin molecules form channels enabling free, non-selective, passive transport of ions, water, small-molecule substances and enzymes. As a consequence, the channels disrupt the protective barrier of the cell membrane and destroy the integrity of the target cell. The immune synapse mediates the release of granzyme B into endosomes in the target cell and ultimately into the target cell cytosol. Granzyme B will initiate apoptosis both by direct cleavage of Caspase 3 and by the cleavage of Bid. Antibody-dependent cellular cytotoxicity is another weapon in the immune arsenal where Fc-receptor bearing effector cells such as Natural Killer cells can recognize and kill antibody-coated target cells expressing tumor or pathogen derived antigens on their surface.

### Regulated Cell Death

There are many different occasions when the cell might have a reason to commit a form of suicide. For example; during embryogenesis for example every child has webbed fingers but at 6-14 weeks of gestation a specific cell death program starts and the interdigital pads regress. Regulated cell death is generally divided into three types but there are additional rare types of regulated cell death that fall between these types. In this invention we have included features from the general types of regulated cell death but do not exclude the use of the rarer types of cell death.

### Type I Apoptosis

The removal of faulty cells is a constant process in our bodies with about a million cells being recycled every second. It is essential for many processes including the elimination of infected or transformed cells, a properly functioning immune system and organismal development. Hallmarks of apoptosis include degradation of DNA, disassembly of the cytoskeleton and nuclear lamina, cellular blebbing, formation of apoptotic bodies and phagocytosis. Importantly there is no leakage of cellular content into the intracellular space thus not inflammatory in contrast to necrosis. It is the generally divided into two pathways: extrinsic and intrinsic. Taken together there are hundreds of genes involved in apoptosis and the interprotein balance decide the fate of the cell. During all stages there are proteins driving apoptosis and other proteins that inhibit those. But whence the final executive caspase has been activated the cell reach a moment of no return and dead is inevitable. In a suicide switch any of the genes regulating apoptosis can be considered. The various genes and gene families are differently expressed in different cell types why a one switch to kill all cells it not our focus, rather a switch for each cell type. In immune cells for example the Bcl-2 family is the dominant drivers regulating survival and apoptosis. In embryonic stem cells upstream regulator p53 is the main inducer of apoptosis. A version of a safety switch is the holy grail of cellular therapy, and many companies are trying to develop their own version. Most of these endeavours focus on the initiating caspases but so far no one has been able to produce a safe and effective switch.

The extrinsic pathway is activated by the binding of extracellular ligands to the death receptors on the cell surface. The death receptors e.g., tumor necrosis factor receptor, share a cytoplasmic domain called the death domain. The death domain transmits the death signal from the cell surface to the intracellular signaling pathways. Adaptor proteins bind to the domain recruiting other adaptor proteins leading to the formation of the death-inducing signaling complex leading to the auto-catalytic activation of procaspase-8. Once activated caspoase-8 will induce the executing caspase cascade. During all steps of this entire cascade inhibitory proteins can block and prevent the final killing of the cell. The intrinsic pathway is activated by cellular stress i.e., DNA damage, hypoxia or any other of an array of intracellular stimuli. This will alter the balance between the pro and antiapoptotic family members of the Bcl-2 protein family in favour of apoptosis. This family of proteins are very significant since they determine if the cell commits to apoptosis or abort the process (FIG. 2). All approximately 20 members of the Bcl-2 family carry Bcl-2 family (BH) domains by which they interact with each other. Whence the proapoptotic members are dominating the mitochondrial membrane is perforated and there is a release of proapoptotic proteins from the intracellular space. These proteins including cytochrome c which in the presence of ADP binds and activates apaf-1 and procaspase-9 forming the apoptosome. The apoptosome formation can be inhibited by the binding of hsp70 and hsp90 to Apaf-1. The apoptosome initiate cleavage of the procaspase-9 into its active form instating the executory caspase cascade. Caspase-9 is approximately 2000 times more active bound to the apoptosome compared with soluble caspase-9. Inhibitor of apoptosis proteins (IAPs) inhibit activated caspases and are the very last checkpoint before cell death. To date, eight mammalian IAPs have been identified: BIRC1 (NAIP/NLRB), BIRC2 (cellular IAP1/cIAP1/human IAP2), BIRC3 (cellular IAP2/cIAP2/human IAP1), BIRC4 (X-linked IAP/XIAP), BIRC5 (survivin), BIRC6 (apollon/BRUCE), BIRC7 (livin/melanoma-IAP, also called ML-IAP/KIAP), and BIRC8 (testis-specific IAP/Ts-IAP/hILP-2). They all share a baculovirus IAP repeat (BIR) domain and most contain a RING domain that functions as an E3 ligase. IAPs such as X-IAP directly inhibit effector caspases, especially caspase 9, whereas c-IAPs modulate cell survival by ubiquitylation of substrates such as ribosome-inactivating protein (RIP) and proteins in the NF-κB pathway. IAPs block apoptosis induced by a variety of stimuli, including Fas, TNF-α, ultraviolet (UV) irradiation, and serum withdrawal. IAPs themselves are inhibited by two mitochondrial proteins named Smac/Diablo and HtrA2/Omi, which are released into the cytosol during the intrinsic and some extrinsic apoptotic programs. Once the initiating caspases (Caspase-8 and -9) have been activated they cleave and activate the executive caspases. These exist in the cell as preformed but inactive homodimers with a short prodomain. Following cleavage mediated by an initiator caspase they act directly on specific cellular substrates to dismantle the cell as well as activating downstream death mediators such as caspase-activated deoxyribonuclease. They also cross talk between the two pathways activating the upstream regulators of the other pathway. Before the DNA is shredded the cell will initiate the expression of “find me” and “eat me” signals recruiting phagocytes to initiate phagocytosis before the apoptotic bodies erupt.

### Type II Autophagy

Autophagy literally translating to self-eating, plays critical roles during embryonic development and is essential for maintaining cell survival, tissue homeostasis, and immunity. Importantly, dysfunctional autophagy has been linked to cancer, infectious diseases, neurodegeneration, muscle and heart diseases, as well as aging. Accumulating evidence demonstrates that autophagy is also critical for stem cell function.

Autophagy is a fundamental cellular process by which cells sequester intracellular constituents, including organelles and proteins, that are delivered to lysosomes for degradation and recycling of macromolecule precursors. The process of autophagy is evolutionarily conserved from yeast to mammals and serves as an essential adaptation mechanism to provide cells with a source of energy during periods of nutrient deprivation and metabolic stress. Under homeostatic conditions, cells maintain a constitutive basal level of autophagy as a method of turning over cytoplasmic content. Autophagy can also be induced in response to cellular stresses such as nutrient deprivation, oxidative stress, DNA damage, endoplasmic reticulum stress, hypoxia, and infection.

The hallmark of autophagy is the formation of double membraned vesicles containing cytoplasmic constituents within the cell known as autophagosomes. Autophagy is a multi-step process of sequential events including induction, nucleation of a phagophore structure, maturation of the autophagosome, autophagosome fusion with the lysosome, and the degradation and recycling of nutrients. The execution of autophagy is dependent on the formation of several key protein complexes and two ubiquitin-like conjugation steps. Initial studies performed to characterize key players in the autophagy pathway were carried out in yeast and identified a family of autophagy-related genes, referred to as Atg, which encode for autophagy effector proteins. Autophagy is inhibited by mTOR a master regulator of cell growth and metabolism. mTOR is also an upstream regulator of apoptosis. There is a significant amount of cross talk between apoptosis and autophagy. The autophagy program can both inhibit and initiate apoptosis depending on the severity of nutrient starvation. It is also a backup in a cell where the apoptotic program is faulty

### Type III Necrosis

While apoptosis is immunologically silent i.e., will not induce an immunological response, necrosis induces a strong immunological response. The necrotic cell will swell up, the plasma membrane becoming destabilized resulting in the release of potentially harmful cellular content and the induction of inflammation. Recent studies have shown that necrosis not only occurs as a response to an accident such as a wound or venomous bite but can also be the result of a cellular program. The different versions of programed necrosis described to date all involve a specific stimulation and all result in the release of entire cellular contents, programmed necrosis also has a specific end response: release of cytokines. However, the field of programmed necrosis is new, and much is still not known. There are various forms of programmed necrosis most sharing parts of their program with apoptosis and/or autophagy. Some forms are still not properly defined as of yet. Necroptosis occurs when death receptor ligands bind to the cell, but the extrinsic pathway is not properly activated. It is a very organized program under strict control through the RIPK1-RIPK3 signaling pathway. Pyroptosis is primarily seen in inflammatory cells such as macrophages. The hallmarks of pyrotopsis are the activation of caspase-1 leading to a massive release of IL-1b and IL-18 and the activation of gasdermin D. Activated gasdermin D will oligomerize and form a membrane pore in the plasma membrane leading to cell swelling, osmotic lysis and release of cellular content including the newly synthesized IL-1b and IL-18. Though the cell dies in a necrotic way they also display features of apoptosis including DNA fragmentation and nuclear condensation. There are more rare forms of cell death most showing one or more feature of all three types of programmed cell death but not falling into any one of them. Entosis, killing via cannibalism. Methuosis a form of necrosis where the cytoplasm is displaced with large fluide filled vacuoles derived from macropinosomes. In this invention we have focus on the tree most common types of programmed cell death and specifically apoptosis.

### Chemically Inducible Promoters

Promoters are formed by a specific combination of transcription binding sites upstream of the transcription start site. This combination will determine the composition of the transcription complex thereby determine the timing and quantity of gene expression. Most common promoters are permanently active and thus referred to as constitutive promoters. However, gene expression is not static, genes are constantly up or down regulated depending on internal and external events. Chemically inducible promoters are promoters induced by an extracellular molecule. Most have been found in bacteria and yeast where they control a process where the cell obliterates the inducing molecule.

Tet on/Off

First identified in gram negative bacteria the Tet system is the most used inducible expression system. Principally, one or more Tet operon sequences are introduced in the promotor of the gene on interest. From another gene the transrepressor (tetR) is expressed. TetR form a dimer which will bind to the Tet operon sequence and block expression. When tetracycline is added, it will bind to the TetR dimers and cause a conformational change releasing the tetR from the operon and induce gene expression. This system has since its discovery in the early 1980s been further developed to function as an on or off switch. By fusing the TetR to the VP16 activation domain a chimeric transactivator (tTA) was formed. The transactivator will bind to the operon to induce gene expression. Since the original report of the Tet switch, several modifications have been reported. These include the use of a repressor to block basal transcription and the fusion of a repression domain to the TetR to generate a silencer molecule.

### Tamoxifen

Nuclear steroid hormone receptors are modular proteins. Tamoxifen inducible gene expression systems take advantage of the ability to fuse ligand binding domains of steroid hormone receptors, in this case the estrogen receptor, to specific DNA binding domains (DBD) to activate expression of a gene of interest only in the presence of ligand. Most commonly used to control site specific recombination, this system can also be used for transcriptional activation. Discovery of specific mutations in the estrogen receptor ligand binding domain (ERBD) that preserved high affinity binding to the anti-estrogen 4-hydroxy tamoxifen but decreased affinity for endogenous estrogens allowed these systems to be employed in mammals without the presence of the endogenous ligand stimulating inappropriate activity of the chimeric protein. In addition to fusing the ERBD to a specific DBD, addition of strong transactivating domain(s), such as the VP16 activation domain, can result in robust gene expression only in the presence of ligand.

### Cumate

The Cumate on/off system is based on a similar principle as the Tet on/off. A naturally occurring p-cmt and p-cym operon control cumic alcohol dehydrogens responsible for the degradation of cumate in Pseudomonas putida. A repressor is bound to the operon but is released in the presence of cumate. Like in the tet system, the cumate system has been manipulated using various activating and repressing elements to produce a stable on/off system.

Van on/Off

Caulobacter cresentus is a gram negative, oligotrophic freshwater bacterium. It plays an important role in the carbon cycle by disposing of the soluble phenolic intermediates such as vanillic acid. Vanillic acid is a byproduct from fungal oxidative cleavage of lignin originating from decaying plant material. It is a common food additive (FAO/WHO expert committee on Food Additives, JECFA no. 959). In conclusion vanillic acid is a safe and physiologically inert gene switch inducer. The Van on/off system depends on a structure with a repressor binding to operons upstream of the transcription start site much like the tet-system. By fusing the Van-repressor with a transcriptional repressor the result is a repressive element shutting down expression when bound to the operon sequence. When vanillic acid is added to the medium it will bind the repressor inducing conformational changes leading to the release of the repressor from the DNA and subsequentially gene expression. The drawback of using vanillic acid as the instigating agent is that it is a highly common food additive that the patient would need to be very careful to avoid.

### Mph(R)

Macrolide such as erythromycin, clarithromycin, and roxithromycin are a group of broad-spectrum antibiotics against gram negative bacteria. Recently a macrolide inactivating 2-phosphotransferease I (mph(A)) was cloned from E choli. The expression of mph(A)) is controlled by a repressor which binds to an operon sequence in the promoter. By fusing the repressor to a KRAB repressor it has been shown to function side by side with the Tetracycline inducible system in human cell lines.

### Alca

AlcA is another repression-operon based system originating from Aspergillus nidulans where the ethanol utilization pathway is upregulated from the ethanol-stabilized AlcR activator bind to the AlcA promoter. It has been utilized in plant cells and tested in E. Coli. It has however not been tried in a human system. There are a few other alcohol induced promoters described briefly in literature including P450IIE1 a microsomal P450 enzyme found in the human liver. Alcohol might be difficult for the patient to avoid, however this switch could be usefull in an in vitro setting.

### DIG

Digoxin is one of the oldest cardiovascular medications used today, it was initially approved by FDA in 1945. It is a steroid like glycoside which bind to and inhibit the activity of the ubiquitous cell surface enzyme Na(+), K(+)-ATPase. A biosensor combining a ligand binding domain fused to a transcription factor and a trans-activator/repressor can effectively induce gene expression. In the digoxin system the complex is stabilized and active in the presence of digoxin but is degraded when these is no ligand present. Thus, reducing the risk of gene leakage.

The invention relates to a safety switch with low levels of constitutively expressed anti-apoptotic protein such as BCL2A1 and inducible expression of pro-apoptotic factors, such as BBC3 or BCL2L11, that allows directed suicide of the hSync transfected cells. By expressing low levels of one or more anti-apoptotic genes, we can increase the viability of the cells even if they enter a hostile environment such as the tumor microenvironment. Also, it is a way to buffer any leakiness from the chemically inducible promoter whereby the pro-apoptotic genes are expressed. Since the promoter(s) controlling expression of the pro-apoptotic genes are very strong, the massive amount of protein produced when we add the initiating agent will override the small amount of anti-apoptotic protein. As there is plenty of room on the hSync we have the possibility to add two or more pro-apoptotic genes under the chemically inducible promoter. By choosing pro-apoptotic proteins with affinities to different anti-apoptotic proteins we can ensure that the cell has no ability to counteract the initiated suicide-switch. Proteins that can be induced in the kill switch include but is not restricted to the Bcl-2 family. Our plan is to build a set of suicide switches suitable for a range of target cell types.

Presently there is no functioning safety switch in use in any cellular therapy capable of shutting down therapeutic cells when desired. Thus, if there is an adverse effect to the cellular therapy there is no mechanism to remove the therapeutic cells. Many are trying to develop such a system but are constrained in their attempts due to the limited space available on vectors used for cellular therapies. Prior to this invention, no one has attempted such an advanced safety switch.

The Bcl-2 family of proteins is a group of proteins located at the mitochondrial membrane. They are in a constantly shifting balance deciding the fate of the cell. They are divided into three groups, anti-apoptotic, pro-apoptotic pore formers and pro-apoptotic BH3-only. All members of the Bcl-2 family contain a BH3 domain, one of four BH domains involved in the interaction between the family members. As long as an anti-apoptotic protein is bound to the proapoptotic pore-forming proteins the cell survives. Whence the pro-apoptotic BH3-only proteins increase in concentration they break the interaction and release the pro-apoptotic pore forming proteins to initiate apoptosis. This is a very complex web of interactions where the affinity between the members is important. The expression of Bcl-2 family members differs greatly with cell type. Thus, switches will be designed to function in the desired target cell type. Designing the switches, the affinity between the family members needs to be considered. If one want to inhibit a leaky switch expressing NOXA1 then Mcl-1 or BCL2A1 would be the best options since the affinity between them are significantly higher than between for example NOXA1 and BCL2L1. The same is true for the broader group of proteins involved in the apoptotic cascade. It is no use just adding an inhibitory protein if it will not bind to the exact protein that is used for the induction of apoptosis. The switch also needs to balanced in regards to gene expression. Trifling with genes regulating cell survival can have some unexpected results. For example, an Extreme overexpression of BCL2 will surprisingly lead to apoptosis rather than increased survival. Probably because an unregulated expression of BCL2 could result in cancer. A number of apoptic genes have been transfected into T-cell using vectors in order to investigate their effect on apoptosis in this specific cell type. Surprisingly a massive co-transfection with multiple proapoptotic genes did not have a stronger induction of apoptosis compared to the single transfections. However, the co-transfection of anti-apoptotic BCL2A1 and BIM again highlight the importance of leveling the gene expression. Highly expressed BCL2A1 will rescue the cells from the effects of BIM.

TABLE 1
Table of proteins in the BCL-2 family.

| | | Accession | | |
|---|---|---|---|---|
| Gene name | Also known as | number | NCBI Gene ID | Function |
| BCL-2 | PPP1R50 | NG_009361.1 | 596 | Anti-apoptotic |
| BCL2L1 | BCL-XL | NG_029002 | 598 | Anti-apoptotic |
| BCL2L2 | BCL-W | NM_004050.5 | 599 | Anti-apoptotic |
| | | NM_001199839.2 | | |
| BCL-A1 | Bfl-1 | NG_029487 | 597 | Anti-apoptotic |
| MCL1 | BCL2L3, EAT, | NG_029146 | 4170 | Anti-apoptotic |
| | TM | | | |
| BAX | BCL2L4 | NG_012191 | 581 | Pro-apoptotic |
| | | | | pore-formers |
| BAK1 | CDN1, BCL2L7 | NM_001188.4 | 578 | Pro-apoptotic |
| | | | | pore-formers |
| BOK | BOKL, BCL2L9 | NG_029488 | 666 | Pro-apoptotic |
| | | | | pore-formers |
| BCL2L13 | BCL-rambo | NM_015367.4 | 23786 | Pro-apoptotic |
| | | | | pore-formers |
| BAD | BBC2, BCL2L8 | NM_004322.3 | 572 | Pro-apoptotic |
| | | NM_032989.3 | | BH3-only protein |
| BID | FP497 | NM_001196.4 | 637 | Pro-apoptotic |
| | | NM_197966.3 | | BH3-only protein |
| | | NM_197967.2 | | |
| BIK | BP4, NBK, BIP1 | NM_001197.5 | 638 | Pro-apoptotic |
| | | | | BH3-only protein |
| BCL2L11 | BIM, BAM, BOD | NG_029006 | 10018 | Pro-apoptotic |
| | | | | BH3-only protein |
| BMF | | NM_001003940.2 | 90427 | Pro-apoptotic |
| | | | | BH3-only protein |
| HRK | DP5, HARAKIRI | NM_003806.4 | 8739 | Pro-apoptotic |
| | | | | BH3-only protein |
| PMAIP1 | NOXA | NM_001382617.1 | 5366 | Pro-apoptotic |
| | | | | BH3-only protein |
| BBC3 | PUMA, JFY1 | NG_031991 | 27113 | Pro-apoptotic |
| | | | | BH3-only protein |

### BCL2L11-Bimₛ

Bcl2L11 or Bim (the B cell lymphoma 2 interacting mediator) is a BH3-only proapoptotic member of the Bcl-2 family. It will activate Bax which will in turn lead to pore formation in the mitochondrial outer membrane and activation of the caspase cascade. Precisely how Bim instigates Bax activity is not fully understood, it can either be through direct interaction with Bax or via neutralization of Bcl-2. In T-cells Bim plays a very important role in terminating the acute immune response but also during development. Mice with T-cell specific Bim KO show abnormal thymocyte development. Bims is the shortest isoform of the regular isoforms of Bim and is the most effective in introducing apoptosis compared to the two longer isoforms and is upregulated in self-reactive thymocytes wherein it orchestrates clonal deletion.

### BBC3-Puma

BBC3, or Puma, is a proapoptotic member of the Bcl-2 protein family. This protein plays a significant role in p53-mediated cell death, but also in p53-independent events such as cell starvation. During activation of the intrinsic apoptotic cascade, Puma will bind to pro-survival family members and break their association with Bax thus instigating mitochondrial pore formation. During the clearance of T-cells after the immune response it is Puma, together with Bim, which orchestrate the apoptotic cascade.

### BCL2A1

BCL2A1 is a pro-survival gene mainly expressed within the hematological system where it facilitates the survival of immune cells. In T-cells the activation of the TCR leads increased expression of BCL2A1. BCL2A1 functions by binding to and inhibiting the pro-apoptotic members of the Bcl-2 protein family. Compared with the other pro-survival members, BCL2 and Bcl-XI, BCL2A1 is more facilitating cell survival rather than driving it. A BCL2A1 knock-out mouse model has reduced but not abolished immune cells, while upregulation of BCL2A1 indicates that BCL2A1 may contribute to tumor progression but is not tumorigenic by itself.

### Caspase 9

The caspase superfamily is the main effector of the apoptotic cascade. Upstream caspases get activated by the apoptotic machinery and in turn activating downstream caspases. At every step there are inhibitors which control the cascade. In the end caspase three is calved of and activated leading to the dismantlement of the cellular structure. Caspase 9 is the initiating caspase downstream of the intrinsic pathway. It is synthesised as procaspase-9 containing a caspase activation domain (CARD) at the N-terminus. It binds to apaf-1 in the apoptosome where it dimerizes and is activated. Compared to most other caspases Procaspase-9 have the ability to autoactivate. Caspase 9-1-thymocytes are rescued from activation of the intrinsic pathway but can still be killed by ligand binding to death receptors. Caspase 9 has rendered great interest in the Car-T field since it is presently the best described and commonly used kill switch on the market. The principal behind the technique is that by fusing caspase-9 to a binding domain. This allows caspase 9 to dimerize and be activated in the presence of a small molecule. This system works in vitro and in mice with different levels of apoptosis achieved. The first round of clinical trials however was stopped by the FDA i.e., serious adverse effects from the molecule itself.

In this invention we give two examples on suicide switches, a simple switch where caspase 9 is under the control of a tetracycline inducible promoter. It is surprisingly effective with a significant loss off cells already after 48 hours after induction. And a second complex switch where BCL2A1 is constantly expressed under the weak promoter PGK. BCL2A1 was chosen since it has a strong affinity for BBC3 and BCL2L11. BBC3 and BCL2L11 are powerful proapoptotic genes effectively activating BAX thus activating the apoptotic cascade. They are expressed under the tetracycline controlled strong promoter CMV and can be induced in a dose dependent manner. In an animal tumor model where the complex safety switch was introduced into a tumor cell line there was a striking loss of tumor cells carrying the hSync after the animals were fed tetracycline over a period of time. These switches are examples of genetic combinations that can be used to induce cell death we propose that with the proper considerations any combination of pro and anti-apoptotic genes could be considered for an hSync.

Use of microRNAs as proapoptotic factors' induction in tumor cells through miRNA has been extensively studied. The biphasic mode (up- and down-regulation) of miRNA expression in apoptosis and other cancer processes has already been determined. The findings of these studies could be utilized to develop potential therapeutic strategies for the management of variousb-cancers. Kashyap et al (2018. Mol. Diag. & Ther 22:170-201) critically describes the oncogenic and tumor suppressor role of miRNAs in apoptosis and other cancer processes, therapy resistance, and use of their presence in the body fluids as biomarkers.” (HYPERLINK “https://link.springer.com/journal/40291” volume 22, pages 179-201.)

| IP Sequence Number | Name of Sequence |
|---|---|
| SEQ ID NO: 1 | CCR4 |
| SEQ ID NO: 2 | CCR6 |
| SEQ ID NO: 3 | CXCR4 |
| SEQ ID NO: 4 | CCR7 |
| SEQ ID NO: 5 | CXCR3 |
| SEQ ID NO: 6 | CX3CR1 |
| SEQ ID NO: 7 | porcine teschovirus-1 2A (P2A) |
| SEQ ID NO: 8 | thosea asigna virus 2A (T2A) |
| SEQ ID NO: 9 | equine rhinitis A virus 2A (E2A) |
| SEQ ID NO: 10 | foot and mouth disease virus 2A (F2A) |
| SEQ ID NO: 11 | CKN BA Pr_FOR |
| SEQ ID NO: 12 | CKN BA Pr_REV |
| SEQ ID NO: 13 | CCR6_FOR |
| SEQ ID NO: 14 | CCR6_REV |
| SEQ ID NO: 15 | 186HygRev |
| SEQ ID NO: 16 | 2010AttR_L |
| SEQ ID NO: 17 | PuroRev |
| SEQ ID NO: 18 | CCR6_JxnF |
| SEQ ID NO: 19 | attPUP |
| SEQ ID NO: 20 | attPDWN |
| SEQ ID NO: 21 | CAPFOR13174 |
| SEQ ID NO: 22 | CAPREV13 |
| SEQ ID NO: 23 | BSR170Rev |
| SEQ ID NO: 24 | PuroRev |
| SEQ ID NO: 25 | Litmus38Prim |
| SEQ ID NO: 26 | ZeoRev |
| SEQ ID NO: 27 | GLOTOX_attLJxF |
| SEQ ID NO: 28 | 2010AttR_R_Hyg |
| SEQ ID NO: 29 | trCD34 full sequence with both ends |
| SEQ ID NO: 30 | trCD34 actual truncated sequence |
| SEQ ID NO: 31 | trCD34 3′ end fragment |
| SEQ ID NO: 32 | trCD34 5′end fragment |
| SEQ ID NO: 33 | CCR4 |
| SEQ ID NO: 34 | CCR6 |
| SEQ ID NO: 35 | CD14 |
| SEQ ID NO: 36 | TrCD14 |
| SEQ ID NO: 37 | CD19 |
| SEQ ID NO: 38 | TrCD19 |
| SEQ ID NO: 39 | CD20 |
| SEQ ID NO: 40 | TrCD20 short |
| SEQ ID NO: 41 | TrCD20 |
| SEQ ID NO: 42 | BCL2 |
| SEQ ID NO: 43 | MCL1 |
| SEQ ID NO: 44 | BAX |
| SEQ ID NO: 45 | BCL2L11 |
| SEQ ID NO: 46 | BID |
| SEQ ID NO: 47 | PMAIP1 |
| SEQ ID NO: 48 | BBC3 |
| SEQ ID NO: 49 | IL-2 |
| SEQ ID NO: 50 | IL-7 |
| SEQ ID NO: 51 | IL-12A |
| SEQ ID NO: 52 | IL-12B |
| SEQ ID NO: 53 | IL-15 |
| SEQ ID NO: 54 | IL-18 |
| SEQ ID NO: 55 | IL-21 |
| SEQ ID NO: 56 | IFNA2 |

### General

It should be understood that any feature and/or aspect discussed above in connections with the compounds according to the invention apply by analogy to the methods described herein.

### Definitions

Unless expressly stated, the terms used herein are intended to have the plain and ordinary meaning as understood by those of ordinary skill in the art. The following definitions are intended to aid the reader in understanding the present invention but are not intended to vary or otherwise limit the meaning of such terms unless specifically indicated.

As used herein, the singular forms “a,” “and,” and “the” include plural referents unless the context clearly dictates otherwise. Thus, for example, reference to “a composition” refers to one or mixtures of compositions, and to equivalent compositions and methods known to those skilled in the art, and so forth; reference to “the therapeutic agent” includes reference to one or more therapeutic agents, and equivalents thereof known to those skilled in the art, and reference to a “an assay” refers to a single assay as well as to two or more of the same or different assays, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Where a range of values is provided, it is understood that each intervening value between the upper and lower limit of that range and any other stated or intervening value in that stated range is encompassed within the invention. The upper and lower limits of these smaller ranges may independently be included in the smaller ranges, subject to any specifically excluded limit in the stated range. Where the stated range includes both of the limits, ranges excluding only one of those included limits are also included in the invention.

Unless defined otherwise, all technical and scientific terms used herein have the same meaning as commonly understood by one of ordinary skill in the art to which this invention belongs. All publications mentioned herein are incorporated herein by reference in their entirety for the purpose of describing and disclosing devices, formulations and methodologies which are described in the publication, and which might be used in connection with the presently described invention.

As used herein, the following terms are intended to have the following meanings:

The term “research tool” as used herein refers to any composition or assay of the invention used for scientific inquiry, academic or commercial in nature, including the development of pharmaceutical and/or biological therapeutics. The research tools of the invention are not intended to be therapeutic or to be subject to regulatory approval; rather, the research tools of the invention are intended to facilitate research and aid in such development activities, including any activities performed with the intention to produce information to support a regulatory submission.

The terms “subject,” “individual,” “host” or “patient” may be used interchangeably herein and typically refer to a vertebrate, often a mammal, and in some embodiments, a human. In some embodiments, the subject is a human patient. Appropriate subjects may include, but are not limited to, rodents (mice, rats, etc.), simians, humans, mammalian farm animals, mammalian sport animals, and mammalian pets, but can also include commercially relevant birds such as chickens, ducks, geese, quail, and/or turkeys. A mammalian subject may be human or other primate (e.g., cynomolgus monkey, rhesus monkey), or commercially relevant mammals, farm animals, sport animals, and pets. such as cattle, pigs, horses, sheep, goats, cats, and/or dogs. The subject can be a male or female of any age group, e.g., a pediatric subject (e.g., infant, child, adolescent) or adult subject (e.g., young adult, middle-aged adult or senior adult). In some embodiments, the subject may be murine, rodent, lagomorph, feline, canine, porcine, ovine, bovine, equine, or primate. In some embodiments, the subject is a mammal. In some embodiments, the subject is a human. In some embodiments, the subject may be female. In some embodiments, the subject may be male. In some embodiments, the subject may be an infant, child, adolescent or adult.

Eukaryotes include all nucleated cells, including unicellular and filamentous yeasts, multicellular organisms including animals and plants. In some embodiments the subject is a mammal. In some embodiments, the mammal is a primate.

As used herein, the terms “treatment,” “treating,” and the like, refer to obtaining a beneficial or desired pharmacologic and/or physiologic effect. For purposes of this disclosure, beneficial or desired effects include, but are not limited to, alleviation of symptoms, diminishment of extent of disease, stabilized (i.e., not worsening) state of disease, preventing spread (i.e., metastasis) of disease, delay or slowing of disease progression, amelioration or palliation of the disease state, and remission (whether partial or total), whether detectable or undetectable. The treatment/effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease. “Treatment” can also mean prolonging survival as compared to expected survival if not receiving treatment. “Palliating” a disease means that the extent and/or undesirable clinical manifestations of a disease state are lessened and/or time course of the progression is slowed or lengthened, as compared to not administering the methods of the present disclosure.

A “therapeutically effective amount,” an “effective amount,” or “efficacious amount” means an amount sufficient to effect beneficial or desired clinical results. For example, an effective amount of a composition, when administered to a mammal or other subject for treating a disease, is sufficient to effect such treatment for the disease. The effective amount will vary depending on the composition, the disease and its severity and the age, weight, etc., of the subject to be treated. An effective amount of a composition can be administered in one or more administrations. An effective amount of a composition is an amount that is sufficient to palliate, ameliorate, stabilize, reverse, slow or delay the progression of the disease state.

Compositions and methods described herein include systems involving at least two-components comprising a therapeutic delivery cell and a bioengineered chromosome. The ideal target therapeutic cell, or its precursor cell line (one that can be differentiated into the ideal therapeutic cell), is transfected with the bioengineered synthetic chromosome carrying necessary genetic elements to provide: 1) safety off switches to (a) eliminate the expression from the synthetic chromosome and/or (b) induce apoptosis of the therapeutic cell by induction of pro-apoptotic factors; 2) cellular enhancements that provide the therapeutic cell with optimal features for therapeutic delivery (e.g., tumor homing of a cancer cell therapeutic cell); 3) therapeutic factors to address the disease indication; and 4) selection elements to enrich for the bioengineered therapeutic cells.

This modular chromosome bioengineering approach involves using site directed recombination to genetically engineering the inputs (components such as, e.g., safety switches, chimeric antigen receptors (CARs), therapeutic genes, large genomic regions including intervening sequences, entire metabolic pathways, and elements for cell selection, for example) onto the synthetic chromosome. Multiple genetic inputs can be delivered to the synthetic chromosome either by delivery of one large genetic payload or by sequential delivery of multiple genetic payloads.

A distinct advantage of the presently disclosed compositions and methods is the provision of readily bioengineered synthetic chromosomes that are portable into many cell types to confer many different useful therapeutic activities to recipient cells. The therapeutic agent can be a gene that confers increased and enhanced cell and/or whole animal survival. Increased and enhanced cell survival can be measured by PCR, for example, to detect the presence of the therapeutic cell. Animal survival can be measured by Kaplan Meier survival analysis. In some embodiments, multiple genes can be positioned and/or sequenced and/or coordinately expressed from a synthetic chromosome to confer increased immune cell survival in response to tumor challenge. In one such example, anti tumoral T cells can be easily bioengineered to circumvent the immune escape often exhibited by tumor cells. Tumor cells employ a variety of means to escape recognition and reduce T-cell function; however, this challenge may be circumvented by engineering T-cells to express from a common regulatory control system multiply-loaded factors that inhibit cell cycle arrest response; e.g., expression of genes that code for inhibitors to the immune and cell cycle checkpoint proteins, such as anti-PD-1 (programmed cell death protein 1) and anti-CTLA-4 (central T-Cell activation and inhibition 4). Additionally, or alternatively, the synthetic chromosome can be engineered to provide the entire tryptophan biosynthetic pathway, to counteract tryptophan depletion from tumor microenvironment by the enzyme IDO and combat T cell exhaustion (see infra). The synthetic chromosomes can be engineered to encode siRNAs to inhibit receptor signaling from e.g. CTLA-4 and/or PD-1. The synthetic chromosomes can be engineered to encode therapeutic agents that reverse the inflammatory environment that switches off desirable effector mechanisms (e.g. TGF-b, IL-10), or to provide or replace cytokines such as IL-2. The synthetic chromosomes can be engineered to encode tumor homing factors, growth factors, T cell maintenance and/or activation factors (e.g., IL2, IL12). Thus, from one inducing regulatory control system, multiple gene products can be produced to enhance immune cell function.

“Synthetic chromosomes” (also referred to as “artificial chromosomes”) are nucleic acid molecules, typically DNA, that have the capacity to accommodate and express heterologous genes and that stably replicate and segregate alongside endogenous chromosomes in cells and are subject to the host cell's native DNA replication and repair mechanisms, thereby providing optimal integrity. A “mammalian synthetic chromosome” refers to chromosomes that have an active mammalian centromere(s). A “human synthetic chromosome” refers to a chromosome that includes a centromere that functions in human cells and that preferably has been produced in human cells. In the present context the term Sync is used as an abbreviation for a synthetic chromosome. hSync is used as an abbreviation for a human synthetic chromosome. When the term hSync is used in the Examples herein, it refers to human synthetic chromosome. However, in the specification and figures, the term hSync is used to mean a synthetic chromosome that may be a human chromosome.

“Endogenous chromosomes” refer to chromosomes found in a cell prior to generation or introduction of a synthetic chromosome.

As used herein, “euchromatin” refers to chromatin that stains diffusely and that typically contains genes, and “heterochromatin” refers to chromatin that remains unusually condensed and transcriptionally inactive. Highly repetitive DNA sequences (satellite DNA) are usually located in regions of the heterochromatin surrounding the centromere.

A “centromere” is any nucleic acid sequence that confers an ability of a chromosome to segregate to daughter cells through cell division. A centromere may confer stable segregation of a nucleic acid sequence, including a synthetic chromosome containing the centromere, through mitotic and meiotic divisions. A centromere does not necessarily need to be derived from the same species as the cells into which it is introduced, but preferably the centromere has the ability to promote DNA segregation in cells of that species. A “dicentric” chromosome is a chromosome that contains two centromeres. A “formerly dicentric chromosome” is a chromosome that is produced when a dicentric chromosome fragments. A “chromosome” is a nucleic acid molecule—and associated proteins—that is capable of replication and segregation in a cell upon division of the cell. Typically, a chromosome contains a centromeric region, replication origins, telomeric regions and a region of nucleic acid between the centromeric and telomeric regions. An “acrocentric chromosome” refers to a chromosome with arms of unequal length. In some embodiments, a mammalian acrocentric chromosome is chosen as starting material to begin the process of making the synthetic chromosome.

For purposes of the present disclosure, and with reference to a synthetic chromosome as disclosed herein, by “the synthetic chromosome is stably maintained,” it is meant that the chromosome has been shown to be faithfully conveyed to and remains present in daughter cells over the course of at least 10 cell divisions or more. In some embodiments, the synthetic chromosome is stably maintained over the course of at least 20 cell divisions. In some embodiments, the synthetic chromosome is stably maintained over the course of at least 30 cell divisions. In some embodiments, the synthetic chromosome is stably maintained over the course of at least 40 cell divisions. In some embodiments, the synthetic chromosome is stably maintained over the course of at least 50 cell divisions. In a rough calculation, on average, a mammalian cell completes one cell division in approximately 24 hours (1 day). In a starting culture containing 100 cells, one cell division (or “doubling”) results in 200 cells. Theoretically and mathematically, after 14 doublings (approximately 14 days in this example), the culture would contain over a million cells, if all cells lived. This is a rough estimate, not least because, in actuality, some cells in the culture die before replicating. Furthermore, in the case of transfecting the cells with a synthetic chromosome, not all cells are readily and successfully transfected to take up the synthetic chromosome, nor are all synthetic chromosomes stably maintained over multiple generations of cell division. The synthetic chromosomes of the presently disclosed cellular therapeutic compositions and methods are stably maintained over many generations of cell division and are readily portable/transfected into target cells, addressing several limitations of previous synthetic chromosomes and systems.

For example, commercially available chemical transfection methods are often used to transfect the bioengineered, flow sort purified chromosomes into recipient cell lines. However, T cells are small relative to other cell types, and their cytoplastic space has a limited capacity for the type of endocytosis relied upon in chemical transfections. Therefore, other chemical transfection methods can be used, including various methods of mechanical transfection methods (e.g., microinjection and nano straws). In some embodiments, such as when cells are used that may be more difficult to transfect, magnetic beads may be a preferable way to select and sort cells that have been successfully transfected and taken up the bioengineered synthetic chromosome.

A “telomere” is a region of repetitive nucleotide sequences—in vertebrates, TTAGGG at each end of a chromosome. Telomeres protect the chromosome from deterioration and fusion with neighbouring chromosomes.

The terms “heterologous DNA” or “foreign DNA” (or “heterologous RNA” or “foreign RNA”) are used interchangeably and refer to DNA or RNA that does not occur naturally as part of the genome in which it is present or is found in a location or locations and/or in amounts in a genome or cell that differ from that in which it occurs in nature. Examples of heterologous DNA include, but are not limited to, DNA that encodes a gene product or gene product(s) of interest. Other examples of heterologous DNA include, but are not limited to, DNA that encodes traceable marker proteins as well as regulatory DNA sequences and entire synthetic chromosomes, and the transcription products thereof.

As used herein, a “coding sequence” is a nucleic acid sequence that “encodes” a peptide, polypeptide, or a functional RNA. A coding sequence can be transcribed (e.g., such as when DNA is transcribed to mRNA) and can be translated (e.g., such as when mRNA is translated into a sequence of amino acids forming a polypeptide) in vivo, in vitro or ex vivo, when placed under the control of appropriate control sequences. The boundaries of the coding sequence often are defined by the presence of a start codon at the 5′ (amino) terminus and a translation stop codon at the 3′ (carboxy) terminus. As used herein, the term “gene” can include any DNA or RNA sequence, double-stranded or single-stranded, which encodes, directly or indirectly, a protein or an RNA (including functional RNAs (e.g., tRNAs, small interfering RNAs, or any RNA with an enzymatic activity), or structural RNAs (such as some rRNAs or long non-coding RNAs, for example)). Synthetic, non-naturally occurring nucleic acids, such as protein nucleic acids (PNAs) may be employed and encoded on the hSync synthetic chromosome.

Alternative synthetic, non-naturally occurring nucleic acids may also be used in the compositions and methods described herein. For example, fluorescently labeled Peptide nucleic acids (PNAs) are an artificially synthesized polymer similar to DNA or RNA and can be used for chromosome painting techniques used to visualize the hSyncs of the present disclosure. PNAs are commercially available through a variety of sources, such as, for example, the New England Biolabs (NEB®) SNAP- and CLIP-tag cell-permeable fusion proteins fluorescent substrates. Another example of a fluorescently labeled nucleotide useful in the methods disclosed herein is MANT-ADP (2′-(or-3′)-O—(N-Methylanthraniloyl) Adenosine 5′-Diphosphate, Disodium Salt) available from Invitrogen™.

The term DNA “control sequences” refers collectively to promoter sequences, polyadenylation signals, transcription termination sequences, upstream regulatory domains, origins of replication, internal ribosome entry sites, enhancers, and the like, which collectively provide for the replication, transcription and translation of a coding sequence in a recipient cell. Not all of these types of control sequences need to be present so long as a selected coding sequence is capable of being replicated, transcribed and translated in an appropriate host cell.

“Operably linked” refers to an arrangement of elements where the components are configured so as to perform their usual function. Thus, control sequences operably linked to a coding sequence are capable of effecting the expression of the coding sequence. The control sequences need not be contiguous with the coding sequence so long as they function to direct the expression of the coding sequence. Thus, for example, intervening untranslated yet transcribed coding or non-coding sequences can be present between a promoter sequence and the coding or non-coding coding sequence and the promoter sequence can still be considered “operably linked” to the coding sequence. In fact, such sequences need not reside on the same contiguous DNA molecule (i.e., chromosome), and may still have interactions resulting in altered regulation.

A “promoter” or “promoter sequence” is a DNA regulatory region capable of binding RNA polymerase in a cell and initiating transcription of a polynucleotide or polypeptide coding sequence such as messenger RNA, or transcription of ribosomal RNAs, small nuclear or nucleolar RNAs, functional non-coding regulatory RNAs, inhibitory RNAs (e.g., siRNAs) or any kind of RNA transcribed by any class of any RNA polymerase I, II or III. In some cases, a promoter may be inducible. In some cases, a promoter may be repressible.

“Recognition sequences” are particular sequences of nucleotides that a protein, DNA, or RNA molecule, or combinations thereof (such as, but not limited to, a restriction endonuclease, a modification methylase or a recombinase) recognizes and binds. For example, a recognition sequence for Cre recombinase is a 34 base pair sequence containing two 13 base pair inverted repeats (serving as the recombinase binding sites) flanking an 8 base pair core and designated loxP. Other examples of recognition sequences include, but are not limited to, attB and attP, attR and attL and others that are recognized by the recombinase enzyme bacteriophage Lambda Integrase. The recombination site designated attB is an approximately 33 base pair sequence containing two 9 base pair core-type Int binding sites and a 7 base pair overlap region; attP is an approximately 240 base pair sequence containing core-type Int binding sites and arm-type Int binding sites as well as sites for auxiliary proteins IHF, FIS, and Xis.

A “recombinase” is an enzyme that catalyzes the exchange of DNA segments at specific recombination sites. An integrase refers to a recombinase that is usually derived from viruses or transposons, as well as perhaps ancient viruses. “Recombination proteins” include excisive proteins, integrative proteins, enzymes, co-factors and associated proteins that are involved in recombination reactions using one or more recombination sites. The recombination proteins used in the methods herein can be delivered to a cell via an expression cassette on an appropriate vector, such as a plasmid, and the like. In other embodiments, recombination proteins can be delivered to a cell in protein form in the same reaction mixture used to deliver the desired nucleic acid(s). In yet other embodiments, the recombinase could also be encoded in the cell and expressed upon demand using a tightly controlled inducible promoter.

The hSync includes multiple possible sites for site-directed recombination (See FIG. 3, in which each potential recombination site for insertion of new genetic material is shown as a band on the human synthetic chromosome (hSync)) and loading of genetic components. A vector comprising the therapeutic and/or cellular enhancing elements and including the reciprocal recombination site (attB) is co-transfected with a unidirectional bacteriophage lambda integrase bearing a mutation that enables the integrase function without the presence of normally required helper proteins. In some embodiments, the recombinase is a unidirectional bacteriophage lambda integrase bearing a mutation that enables the integrase function without the presence of normally required helper proteins. In some embodiments, the synthetic chromosome is engineered to contain multiple recombination acceptor sites (e.g., over 50 sites; or between 10 and 100 sites; or, for example, 75 acceptor sites).

Synthetic platform chromosome technology relies on a site-specific recombination system that allows the “loading” or placement of selected regulatory control systems and genes onto the synthetic chromosome. In some embodiments, the synthetic platform chromosome comprises multiple site-specific recombination sites into each of which one or several genes of interest may be inserted. Any known recombination system can be used, including the Cre/lox recombination system using CRE recombinase from E. coli phage P1; the FLP/FRT system of yeast using the FLP recombinase from the 2p episome of Saccharomyces cerevisiae; the resolvases, including Gin recombinase of phage Mu, Cin, Hin, a5, Tn3; the Pin recombinase of E. coli; the R/RS system of the pSR1 plasmid of Zygosaccharomyces rouxii; site-specific recombinases from Kluyveromyces drosophilarium and Kluyveromyces waltii; and other systems known to those of skill in the art; however, recombination systems that operate without the need for additional factors—or by virtue of mutation do not require additional factors—are preferred. In one exemplary embodiment, a method is provided for insertion of nucleic acids into the synthetic platform chromosome via sequence-specific recombination using the recombinase activity of the bacteriophage lambda integrase.

Lambda phage-encoded integrase (designated “Int”) is a prototypical member of the integrase family. Int effects integration and excision of the phage into and out of the E. coli genome via recombination between pairs of attachment sites designated attB/attP and attL/attR. Each att site contains two inverted 9 base pair core Int binding sites and a 7 base pair overlap region that is identical in wild-type att sites. Int, like the Cre recombinase and Flp-FRT recombinase systems, executes an ordered sequential pair of strand exchanges during integrative and excisive recombination. The natural pairs of target sequences for Int, attB and attP or attL and attR are located on the same or different DNA molecules resulting in intra- or inter-molecular recombination, respectively. For example, intramolecular recombination occurs between inversely oriented attB and attP, or between attL and attR sequences, respectively, leading to inversion of the intervening DNA segment. Though wildtype Int requires additional protein factors for integrative and excisive recombination and negative supercoiling for integrative recombination, mutant Int proteins do not require accessory proteins to perform intramolecular integrative and excisive recombination in co-transfection assays in human cells and are preferred for the methods of the present invention.

In some embodiments, a mutant integrase λINTR integrase is used; in some embodiments, the integrase is derived and modified from lambda phage integrase. Transgenes (genes of interest) may be introduced using λINTR integrase-mediated targeting to the synthetic chromosome via attP×attB recombination.

“Ribosomal RNA” (rRNA) is the specialized RNA that forms part of the structure of a ribosome and participates in the synthesis of proteins. Ribosomal RNA is produced by transcription of genes which, in eukaryotic cells, are present in multiple copies. In human cells, the approximately 250 copies of rRNA genes (i.e., genes which encode rRNA) per haploid genome are spread out in clusters on at least five different chromosomes (chromosomes 13, 14, 15, 21 and 22). In human cells, multiple copies of the highly conserved rRNA genes are located in a tandemly arranged series of rDNA units, which are generally about 40-45 kb in length and contain a transcribed region and a nontranscribed region known as spacer (i.e., intergenic spacer) DNA which can vary in length and sequence.

Functional non-coding regulatory RNAs (e.g., siRNAs and antisense RNAs) are also well known and characterized and may be useful in some embodiments of the present disclosure in regulation of expression of coding or non-coding DNA sequences.

A selectable marker operative in the cellular host optionally may be present to facilitate selection of cells containing the synthetic chromosome. As used herein the term “selectable marker” refers to a gene introduced into a cell, particularly in the context of this invention into cells in culture, that confers a trait suitable for artificial selection. General use selectable markers are well-known to those of ordinary skill in the art. In some embodiments, selectable markers for use in a human synthetic chromosome system should be non-immunogenic in the human and include, but are not limited to: human nerve growth factor receptor (detected with a MAb); truncated human growth factor receptor (detected with MAb); mutant human dihydrofolate reductase (DHFR; fluorescent MTX substrate available); secreted alkaline phosphatase (SEAP; fluorescent substrate available); human thymidylate synthase (TS; confers resistance to anti-cancer agent fluorodeoxyuridine); human glutathione S-transferase alpha (GSTA1; conjugates glutathione to the stem cell selective alkylator busulfan; chemoprotective selectable marker in CD34⁺ cells); CD24 cell surface antigen in hematopoietic stem cells; human CAD gene to confer resistance to N-phosphonacetyl-L-aspartate (PALA); human multi-drug resistance-1 (MDR-1; P-glycoprotein surface protein selectable by increased drug resistance or enriched by FACS); human CD25 (IL-2a; detectable by Mab-FITC); Methylguanine-DNA methyltransferase (MGMT; selectable by carmustine); and Cytidine deaminase (CD; selectable by Ara-C). Drug selectable markers such as puromycin, hygromycin, blasticidin, G418, tetracycline, zeocin may also be employed. In addition, using FACs sorting, any fluorescent marker gene may be used for positive selection, as may chemiluminescent markers (e.g. Halotags), and the like.

“Binding” as used herein (e.g., with reference to an nucleic acid-binding domain of a polypeptide) refers to a non-covalent interaction between a polypeptide and a nucleic acid. While in a state of non-covalent interaction, the polypeptide and nucleic acid are said to be “associated”, “interacting”, or “binding”. Binding interactions are generally characterized by a dissociation constant (Kd) of less than 10⁻⁶ M to less than 10⁻¹⁵ M. “Affinity” refers to the strength of binding, increased binding affinity being correlated with a lower Kd.

By “binding domain” it is meant a polypeptide or protein domain that is able to bind non-covalently to another molecule. A binding domain can bind to, for example, a DNA molecule (a DNA-binding protein), an RNA molecule (an RNA-binding protein) and/or a protein molecule (a protein-binding protein).

“Site-specific recombination” refers to site-specific recombination that is effected between two specific sites on a single nucleic acid molecule or between two different molecules that requires the presence of an exogenous protein, such as an integrase or recombinase. Certain site-specific recombination systems can be used to specifically delete, invert, or insert DNA, with the precise event controlled by the orientation of the specific sites, the specific system and the presence of accessory proteins or factors. In addition, segments of DNA can be exchanged between chromosomes, such as in chromosome arm exchange.

A “vector” is a replicon, such as plasmid, phage, viral construct, cosmid, bacterial artificial chromosome, P-1 derived artificial chromosome or yeast artificial chromosome to which another DNA segment may be attached. In some instances, a vector may be a chromosome such as in the case of an arm exchange from one endogenous chromosome engineered to comprise a recombination site to a synthetic chromosome. Vectors are used to transduce and express a DNA segment in a cell. In some embodiments, a delivery vector is used to introduce an expression cassette onto the synthetic platform chromosome. The delivery vector may include additional elements; for example, the delivery vector may have one or two replication systems; thus allowing it to be maintained in organisms, for example in mammalian cells for expression and in a prokaryotic host for cloning and amplification.

The choice of delivery vector to be used to deliver or “load” the multiple regulatory control systems and multiple genes onto the synthetic platform chromosome will depend upon a variety of factors such as the type of cell in which propagation is desired. The choice of appropriate delivery vector is well within the skill of those in the art, and many vectors are available commercially. To prepare the delivery vector, one or more genes under the control of one or more regulatory control systems are inserted into a vector, typically by means of ligation of the gene sequences into a cleaved restriction enzyme site in the vector. The delivery vector and the desired multiple regulatory control systems may also be synthesized in whole or in fractions that are subsequently connected by in vitro methods known to those skilled in the art. Alternatively, the desired nucleotide sequences can be inserted by homologous recombination or site-specific recombination. Typically homologous recombination is accomplished by attaching regions of homology to the vector on the flanks of the desired nucleotide sequence (e.g., cre-lox, att sites, etc.). Nucleic acids containing such sequences can be added by, for example, ligation of oligonucleotides, or by polymerase chain reaction using primers comprising both the region of homology and a portion of the desired nucleotide sequence. Exemplary delivery vectors that may be used include but are not limited to those derived from recombinant bacteriophage DNA, plasmid DNA or cosmid DNA. For example, plasmid vectors such as pBR322, pUC 19/18, pUC 118, 119 and the M13 mp series of vectors may be used. Bacteriophage vectors may include λgt10, Agtl 1, λgt18-23, λZAP/R and the EMBL series of bacteriophage vectors. Cosmid vectors that may be utilized include, but are not limited to, pJB8, pCV 103, pCV 107, pCV 108, pTM, pMCS, pNNL, pHSG274, COS202, COS203, pWE15, pWE16 and the charomid 9 series of vectors. Additional vectors include bacterial artificial chromosomes (BACs) based on a functional fertility plasmid (F-plasmid), yeast artificial chromosomes (YACs), and P1-derived artificial chromosomes, DNA constructs derived from the DNA of P1 bacteriophage (PACS). Alternatively, recombinant virus vectors may be engineered, including but not limited to those derived from viruses such as herpes virus, retroviruses, vaccinia virus, poxviruses, adenoviruses, lentiviruses, adeno-associated viruses or bovine papilloma virus. Alternatively, the genes under control of the regulatory control systems may be loaded onto the synthetic platform chromosome via sequential loading using multiple delivery vectors; that is, a first gene under control of a first regulatory control system may be loaded onto the synthetic platform chromosome via a first delivery vector, a second gene under control of a second regulatory control system may be loaded onto the synthetic platform chromosome via a second delivery vector, and so on.

Using lambda integrase mediated site-specific recombination- or any other recombinase-mediated site-specific recombination—the genes under regulatory control are introduced or “loaded” from the delivery vector onto the synthetic platform chromosome. Because the synthetic platform chromosome contains multiple site-specific recombination sites, the multiple genes may be loaded onto a single synthetic platform chromosome. The recombinase that mediates the site-specific recombination may be delivered to the cell by encoding the gene for the recombinase on the delivery vector, or purified protein or encapsulated recombinase protein delivered to a recipient cell using standard technologies. Each of the multiple genes may be under the control of its own regulatory control system; alternatively, the expression of the multiple genes may be coordinately regulated via viral-based or human internal ribosome entry site (IRES) elements or as pro-peptides responsive to the host cells endogenous processing system (e.g., preproinsulin). Additionally, using IRES type elements or 2A peptides linked to a fluorescent marker downstream from the target genes—e.g., green, red or blue fluorescent proteins (GFP, RFP, BFP)—allows for the identification of synthetic platform chromosomes expressing the integrated target genes. Alternatively, or in addition, site-specific recombination events on the synthetic chromosome can be quickly screened by designing primers to detect integration by PCR.

The vectors carrying the components appropriate for synthetic chromosome production can be delivered to the cells to produce the synthetic chromosome by any method known in the art. The terms transfection and transformation refer to the taking up of exogenous nucleic acid, e.g., an expression vector, by a host cell whether or not any coding sequences are, in fact, expressed. Numerous methods of transfection are known to the ordinarily skilled artisan, for example, by Agrobacterium-mediated transformation, protoplast transformation (including polyethylene glycol (PEG)-mediated transformation, electroporation, protoplast fusion, and microcell fusion), lipid-mediated delivery, liposomes, electroporation, sonoporation, microinjection, particle bombardment and silicon carbide whisker-mediated transformation and combinations thereof; direct uptake using calcium phosphate; polyethylene glycol (PEG)-mediated DNA uptake; lipofection; microcell fusion; lipid-mediated carrier systems; or other suitable methods. Successful transfection is generally recognized by detection of the presence of the heterologous nucleic acid within the transfected cell, such as, for example, any visualization of the heterologous nucleic acid, expression of a selectable marker or any indication of the operation of a vector within the host cell.

An “antigen” (Ag) as used herein is any structural substance which serves as a target for the receptors of an adaptive immune response, TCR or antibody, respectively. Antigens are in particular proteins, polysaccharides, lipids and substructures thereof such as peptides. Lipids and nucleic acids are in particular antigenic when combined with proteins or polysaccharides.

“Effector cell” refers to a cell that carries out a specific activity in response to stimulation. The term effector cell generally is applied to certain cells in the immune system

“Cytolytic cells” refers to a cell capable off capable of destroying other cells.

“Cytolytic T lymphocytes (CTL)” refers to a T cell that normally carries CD8 on the cell surface and that functions in cell-mediated immunity by destroying a cell (such as a virus-infected cell or tumor cell) having a specific antigenic molecule displayed on its surface.

“Antigen stimulation” refers to a B cell or T cell being stimulated T or B cell receptor be recognizing a specific antigen.

In the present context “tumor associated antigen” or “TAA” is antigen that is presented by MHCI or MHCII molecules or non-classical MHC molecules on the surface of tumor cells. As used herein TAA includes “tumor-specific antigen”, which is found only on the surface of tumor cells, but not on the surface of normal cells.

“Expansion” or “clonal expansion” as used herein means production of daughter cells all arising originally from a single cell. In a clonal expansion of lymphocytes, all progeny share the same antigen specificity.

“Memory cells”, currently represented by T and B lymphocytes and natural killer cells, which determine a rapid and effective response against a second encounter with the same antigen.

“Costimulation” refers to a signaling pathway that augment antigen receptor-proximal activation events, and that intersects with antigen-specific signals synergistically to allow lymphocyte activation.

### Sequence Identity

The homology between two amino acid sequences or between two nucleic acid sequences is described by the parameter “identity”. Alignments of sequences and calculation of homology scores may be done using e.g. a full Smith-Waterman alignment, useful for both protein and DNA alignments. The default scoring matrices BLOSUM50 and the identity matrix are used for protein and DNA alignments respectively. The penalty for the first residue in a gap is −12 for proteins and −16 for DNA, while the penalty for additional residues in a gap is −2 for proteins and −4 for DNA. Alignment may be made with the FASTA package version v20u6. Multiple alignments of protein sequences may be made using “ClustalW”. Multiple alignments of DNA sequences may be done using the protein alignment as a template, replacing the amino acids with the corresponding codon from the DNA sequence. Alternatively, different software can be used for aligning amino acid sequences and DNA sequences. The alignment of two amino acid sequences is e.g. determined by using the Needle program from the EMBOSS package (http://emboss.org) version 2.8.0. The substitution matrix used is BLOSUM62, gap opening penalty is 10, and gap extension penalty is 0.5.

As used herein, the term “stem cells” can refer to embryonic stem cells, fetal stem cells, adult stem cells, amniotic stem cells, induced pluripotent stem cells (“iPS cells” or “iPSCs”), or any cell with some capacity for differentiation and/or self-renewal. iPS cells are adult cells reprogrammed to exhibit pluripotent capabilities.

As used herein, the term “adult-derived mesenchymal stem cells” (“MSCs”) refers to cells that can be isolated from bone marrow, adipose tissue, peripheral blood, dental pulp, lung tissue or heart tissue from a non-fetal animal. Human MSCs are known to positively express cell surface markers CD105 (SH2), CD73 (SH3), CD44 and CD90, and do not express cell surface markers CD45, CD34, CD14, CD11b, or HLA-DR. Adult-derived mesenchymal stem cells exhibit plastic-adherence under standard culture conditions, are able to develop as fibroblast colony forming units, and are competent for in vitro differentiation into osteoblasts, chondroblasts and adipocytes. “hMSCs” as used herein refers to human adult-derived mesenchymal stem cells.

The following figures and examples are provided below to illustrate the present invention. They are intended to be illustrative and are not to be construed as limiting in any way.

### BRIEF DESCRIPTION OF THE FIGURES

FIG. 1. Overview of the construction of a human synthetic chromosome.

FIG. 2. Fluorescent in situ hybridization of CHO transfected hSync subclone, L3, derived following limiting dilution cloning of the original clonal isolate. Note that the hSync, with no additional human chromosomes, is present. The modal chromosome number in this cell line is 21.

FIG. 3. Generalized map of one or more chemokine receptor inserts on an hSync. One or more chemokines can be expressed from a synthetic chromosome upon integration of a plasmid vector carrying the chemokines of interest onto the synthetic chromosome. The chemokines may be expressed from independent promoters (A); or may be expressed from bidirectional promoters (B); or may be transcribed from a single promoter with internal ribosomal entry sites (IRES) elements separating the individual cytokine products (C); or encoded as a single transcript from a single promoter and individual chemokines would arise from posttranslational processing at the 2A sites (D); or from a combination of A, B, C and D.

AR: Antibiotics resistance or marker gene; attR and attL: sequence products of a site-specific DNA recombination reaction between attB and attP; INS: insulator element; CR: chemokine receptor; BD promoter: Bidirectional promoter; IRES: internal ribosomal entry site; 2A: self-cleaving peptide element.

FIG. 4. Map of a chemokine receptor insert in hSynC with supporting elements PR: promoter; AR: Antibiotics resistance or marker gene; attR and attL: sequence products of a site-specific DNA recombination reaction between attB and attP; INS: insulator element; CR: chemokine receptor; Marker: marker gene; SE: functional support element(s) for cell.

FIG. 5. Figure X. Map of the hSynC-pkgCCR4_tetCCR6 chromosome attR and attL: sequence products of a site-specific DNA recombination reaction between attB and attP; INS: insulator element; BSR: Blasticidin S deaminase; trCD34: truncated CD34

FIG. 6. hSynC-pkgCCR4_tetCCR6 T cells migrate towards chemokine ligands. Expected result. T cells from healthy human donor PBMCs are activated and expanded with TransAct beads (Miltenyi Biotec) and transfected with hSynC-pkgCCR4_tetCCR6 chromosomes. 1×105 T hSynC-pkgCCR4_tetCCR6 T cells are seeded in the upper well of 5 μm pore size 96 well transwell plates. The lower wells contain the indicated chemokines (10 ng-1 μg/ml) and tetracycline when indicated. Cell migration is followed by microscopy.

FIG. 7. Activated primary T cells express the CCR4 and CCR6 chemokine receptors after electroporation with plasmids. T cells from healthy human donor PBMCs were activated and expanded with TransAct beads (Miltenyi Biotec). Activated T cells were electroporated (Lonza 4D-Nucleofector) with plasmid vectors of CCR4 (CCR4_OHu25514D_pcDNA3.1+/C—(K)-DYK) or CCR6 (CCR6_OHu23447D_pcDNA3.1+/C—(K)-DYK). Protein expression on cell surface and intracellularly (Biolegend intracellular kit) was assayed 72 hours after transfection with anti-CCR4 (1G1) and anti-CCR6 (FAB195P) antibodies and measured with flow cytometry (Miltenyi Quant).

FIG. 8 describes the modularity of this approach to cell and gene therapy: cell+bioengineered synthetic chromosome yields a therapeutic cell composition.

FIG. 9 illustrates the modular approach in which a synthetic chromosome is bioengineered to include any of several therapeutic factors/cellular enhancements and functions (components such as safety switches, chimeric antigen receptors (CARs), therapeutic genes, large genomic regions including intervening sequences, entire metabolic pathways, and elements for cell selection, for example.

FIG. 10 exemplifies the bioengineering of human synthetic chromosome (hSync) by delivering/loading the desired genetic elements onto any of multiple possible sites for site directed recombination (each recombination site (e.g., attP) is shown as a band on the metaphase chromosome) using a vector (comprising the reciprocal recombination site (e.g., attB) as well as the therapeutic and/or cellular enhancing elements) and a unidirectional bacteriophage lambda integrase.

FIG. 11 shows how the cell+hSync cellular therapeutic can be used for oncological applications. In this example, autologous or allogeneic T cells isolated from a sentinel node in the cancer patient and the bioengineered hSynC containing two safety switches (Xist and apoptotic factors BBC3 & BCL2L11), cellular enhancement factors (IL2 and CCR4), a selection element (truncated CD34) and, optionally, other therapeutic factors such as multiple cancer-specific CARs which can be optimized for the specific cancer.

FIG. 12 shows how the cell+hSync cellular therapeutic can be used for orphan genetic disease therapies. This example includes a target MSC therapeutic delivery cell and the bioengineered chromosome containing two safety switches (Xis and apoptotic factors BBC3 & BCL2L11), cell enhancement factors, a selection element (truncated CDXX) and the genomic locus of a wildtype Niemann Pick gene as the therapeutic factor.

FIG. 13 shows the genetic components of a bioengineered chromosome dubbed “OncoSync,” for therapeutic use with autologous sentinel node T cells comprising: (a) a cell selection element (truncated CD34); (b) enhancement factors IL-2, a T cell growth factor and CCR4 for homing to the tumor; and (c) two inducible safety switches (XIST and apoptotic factors BBC3 & BCL2L11). OncoSync is transfected into the patient's tumor-educated T cells to produce the therapeutic cell & gene therapy composition.

### METHODS

### Quality Control

An extensive list is used for release criteria and quality control procedures including in process controls, product integrity and quality testing, safety testing and efficacy testing as described by others previously (Yonghong et al., 2019). Examples of relevant tests are:

### Cell Count Assay

Cells are counted and a rough viability analysis is performed by using trypan blue. It will make it easy to distinguish the live cells from the dead. Both sets of cells are quantified in a microscope.

### Viability Assays

Using flow cytometry one can analyze cell viability in depth using various viability dyes. Annexin V dye will stain the Annexin V that has moved from the intracellular to the extracellular side of the cellular membrane. Propidium Iodine, DAPI and similar stains all stain nucleic acid but are impermeable to live cells. Thus, these nucleic acid stains are a marker of necrotic cells where the cell membrane has broken down.

Mitochondrial stains effectively assess the integrity of the mitochondrial membrane and are thus a good marker of apoptosis. Intact mitochondria retain the dye while apoptotic mitochondria, where the membrane has been perforated, will quickly lose fluorescence.

Caspases can be investigated using various methods. With flow cytometry the cells are first treated with a quiescent substrate of the active caspase. When the substrate is cleaved by active caspase there is a fluorescent signal. Western blot may also be used, the cells are lysed, the lysate run through a gel to separate proteins and an antibody specific for the active caspase, is used in detection.

### T Cell Phenotype

T-cells are phenotyped using flow cytometry and markers typically used are CD3, CD4 and CD8. Additional markers can be added to the panel if there is an interest to further subgroup the cells.

### Sterility Testing

Sterility of the cell media will be analyzed by a GMP compliant CRO company.

### Chromosome Integrity and Genomic Stability

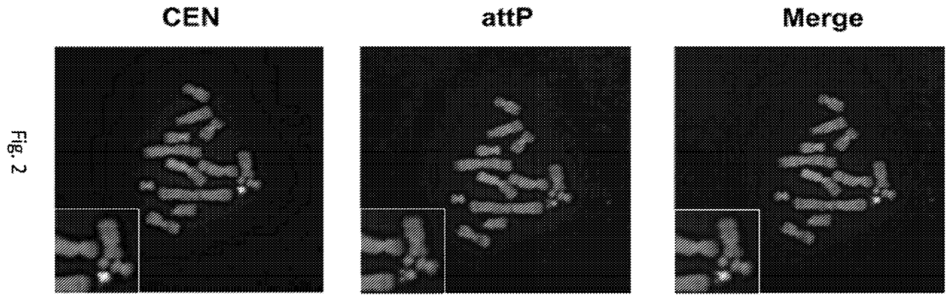

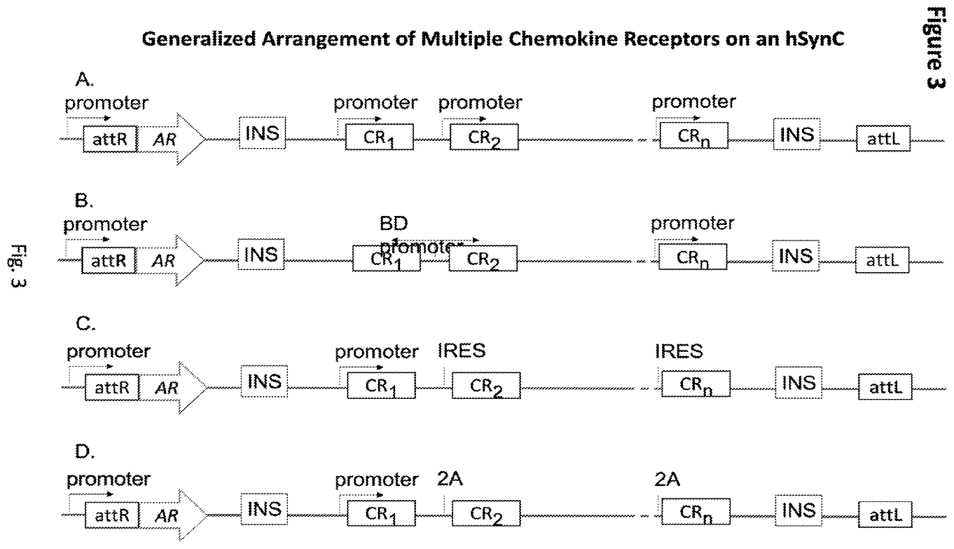

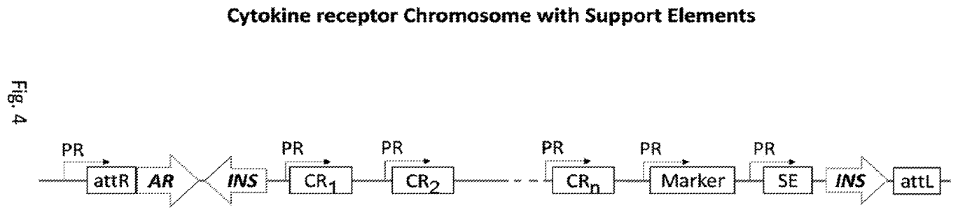

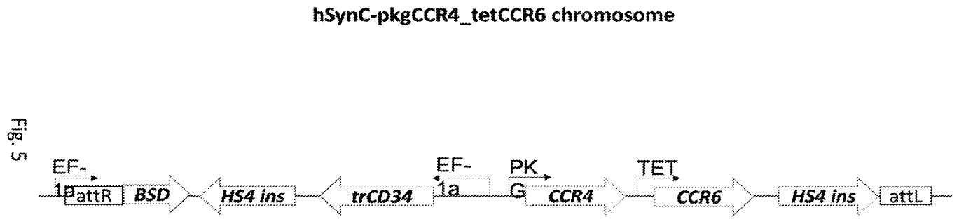

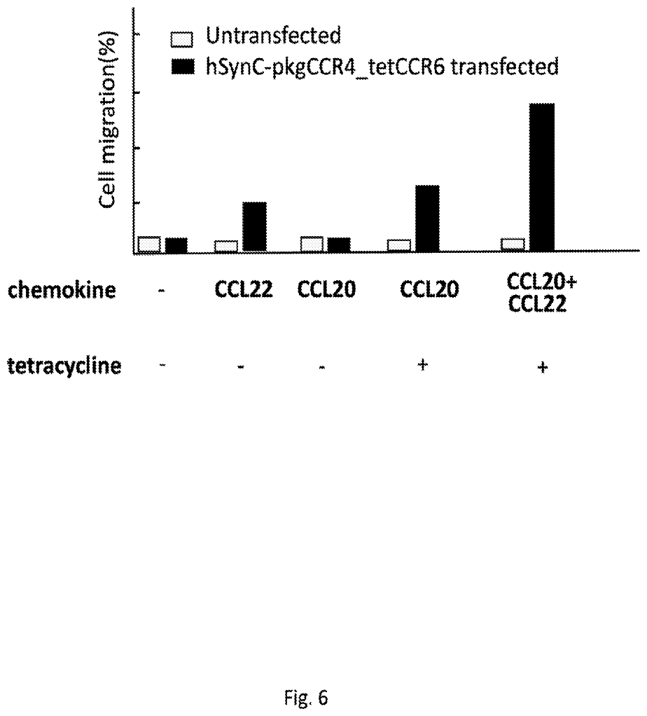

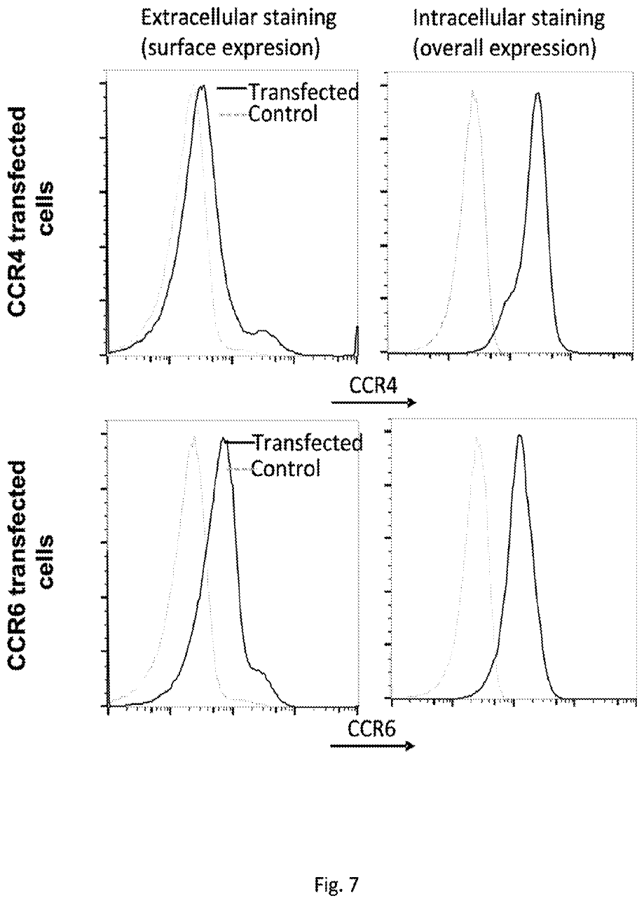

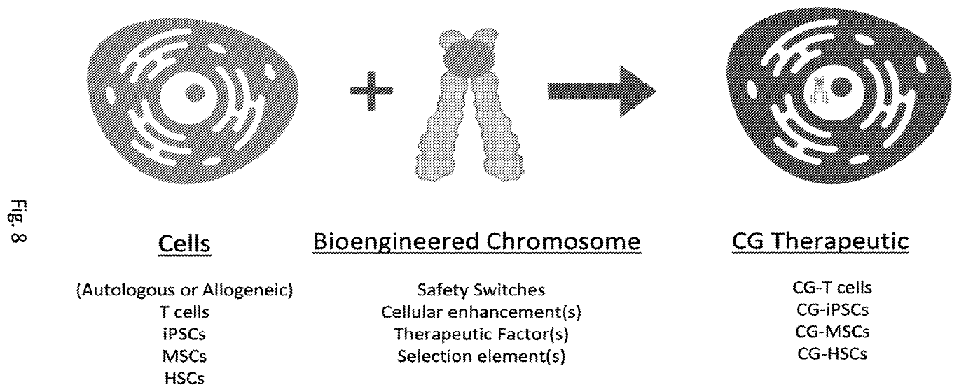

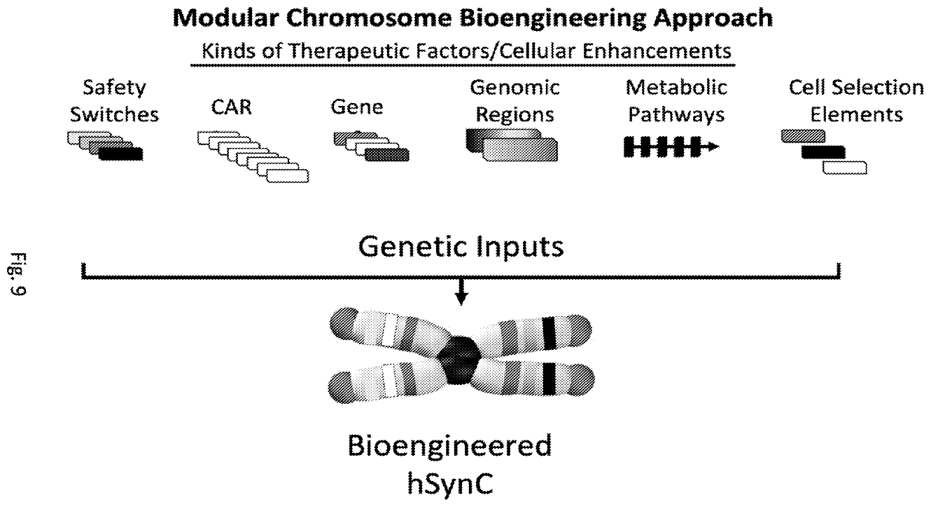

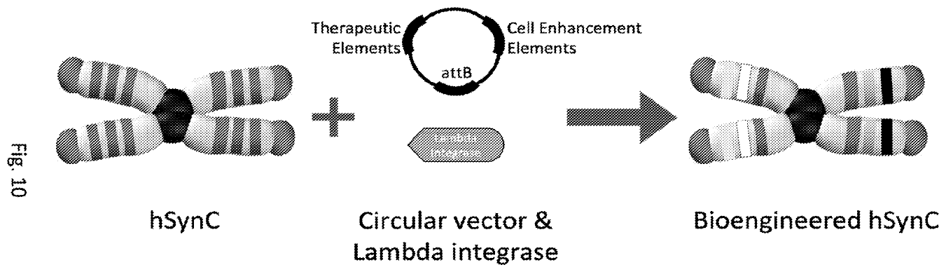

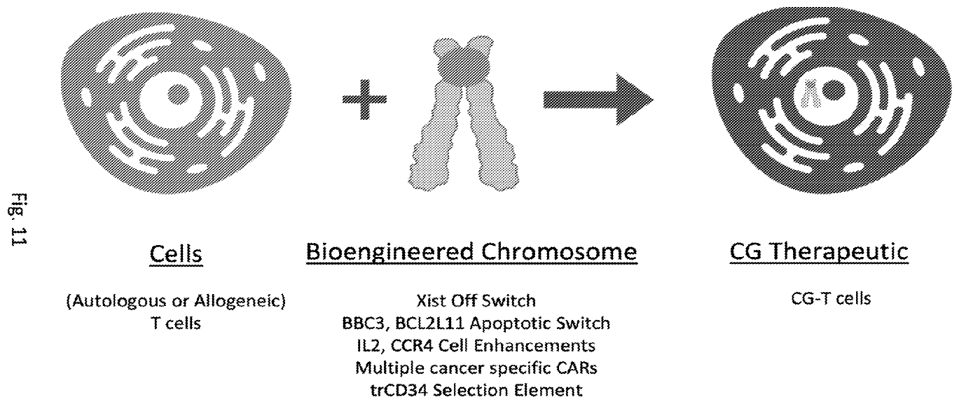

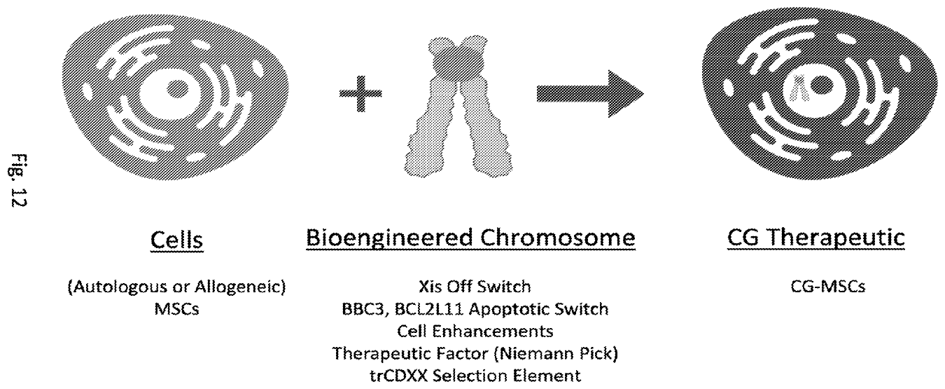

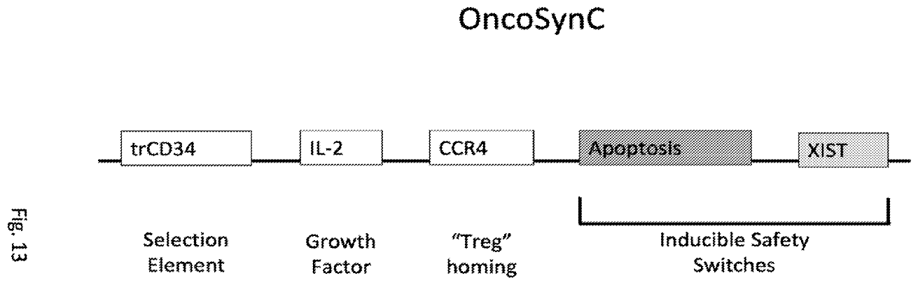

The hSync contains chromosomal structural elements necessary for integrity and stability, i.e., centromeres (FIG. 2 FISH of hSync). Telomeres are necessary for chromosome integrity, acting as caps at the ends of chromosomes, preventing the DNA strand from being detected as a double stranded DNA break and undergoing repair reactions that can lead to chromosome rearrangements. Centromeres are necessary for chromosome stability and are responsible for accurate partitioning to daughter cells at each cell division. In addition to these structural elements, the hSync contains a multitude of lambda virus attP sites, which are not present in eukaryotic genomes, that permit unidirectional integration of therapeutic DNAs. These attP sites are hSycC specific markers. After loading a therapeutic nucleic acid element onto the hSynC, the attP site is replaced by attR and attL sites that flank the therapeutic nucleic acid and are unique to it. Following each engineering or transfer step, assays to ensure the integrity and stability of the hSync and therapeutic nucleic acid(s) are carried out.

### Standard Fluorescent In Situ Hybridization

Metaphase Chromosome Preparation: Metaphase cells are prepared by treating actively dividing cultures with 10 μg/mL Karyomax (Gibco, USA, 15212-012) for 4-12 hours. Metaphase cells are collected by trypsinization, concentrated by centrifugation and treated with 75 mM KCl for 15 min at 37° C. prior to standard fixation in 3:1 methanol:acetic acid. Fixed cells were stored at −20° C. until use.

Generation of labeled probes: Probes for fluorescent in situ hybridization were generated by polymerase chain reaction (PCR) using templates and primers described in Table X. Probes specific for the attP vector sequences (4 individual PCR products) were labeled with biotin-11-dUTP (Roche, Germany, Cat No 11093070910) and alpha satellite centromeric sequences were labeled with digoxigenin-11-dUTP (Roche, Germany, Cat No 11558706910). PCR reactions contained 0.5 ng template, 400 uM each primer, 1× FastStart Taq buffer with MgCl2 provided by the manufacturer (Roche, Germany, Cat No 1232929001) and 0.1 unit FastStart Taq polymerase. For labelling reactions, the dNTP mixture contained dATP, dCTP and dGTP at 200 uM each and dTTP at 130 uM. Labeled nucleotide was added to 70 uM. Control reactions contained only unlabeled nucleotide, all at 200 uM final concentration. dNTP mixtures were prepared from Deoxynucleoside Triphosphate Set (Roche, Germany, Cat No 11277049001). All PCR reactions except for the one generating alpha satellite probe were carried out as follows: 4 min at 95° C., 35 cycles of 95 C for 30 sec, 62° C. for 30 sec and 72° C. for 30 sec, and a final 2 min at 72° C. For alpha satellite probe amplification conditions were identical except the annealing temperature was 52° C. PCR products were assessed by agarose gel electrophoresis before are purified using the Monarch PCR purification kit following the manufacturers recommendation. Probe concentrations are determined using a nanodrop.

Fluorescent in situ hybridization: Metaphase cells are spread on glass slides and aged at 65° C. overnight. Slides are treated with 100 μg/mL RNase A (Sigma, USA, Cat No R4642) for 20 min at 37° C. before being washed 2× at room temperature in 1×PBS. The slides are dehydrated by passing through a room temperature ethanol series (70%, 85%, 100%) for 2 min each and air dried. Metaphase chromosomes are denatured in 70% formamide/2× saline sodium citrate (SSC) at 70° C. for 2 min before being dehydrated by passing through a second ethanol series at −20° C. as described above and being air dried.

Probe mixtures (100 ng/slide of combined biotinylated attP probes with 100 ng/slide of digoxigenin-labeled alpha satellite probe) are combined with 60 μl/slide of Hybrisol VII (MP Biomedicals, USA, Cat No RIST1390). Denatured salmon sperm DNA (Sigma, USA, Cat No D1626) is added to a final concentration of 0.4 mg/mL. The probe mixture is denatured at 75° C. for 10 min before being snap cooled on ice. 60 μL of probe mixture is added to the slide and a coverslip was placed on the slide. The coverslip is sealed with rubber cement. Slides are hybridized overnight at 37° C.

To detect the probe signals, coverslips are removed, and slides are washed 2 times in 2×SSC at 42° C. for 8 minutes each time followed by 2 washes in 50% formamide/2×SSC at 42° C. for 8 minutes each. Slides are briefly rinsed in 1×PBD (18 mM phosphate buffer (30 mM sodium) with 0.01% Triton-X 100, pH 8.0) before being incubated for 1 hour at 37° C. in 1×ISH blocking buffer (Vector Laboratories, USA, Cat No MB-1220). Slides are incubated with Alexa Fluor 488-labeled mouse anti-digoxigenin (Jackson ImmunoResearch, USA, Cat No 200542156) and Alexa Fluor 549-labeled streptavidin (Jackson ImmunoResearch, USA, Cat No 016580084) diluted in 1×ISH buffer for 1 hour at 37° C. Slides are washed 3 times with agitation for 2 minutes each wash in 1× PBD before being incubated for 30 minutes at 37° C. with Alexa Fluor 488-labeled goat anti-mouse IgG (Jackson ImmunoResearch, USA, Cat No 200542156) and biotinylated-anti-streptavidin (Vector Laboratories, USA, Cat No BP-0500) diluted in 1×ISH buffer. Slides are washed as above with 1×PBD. Finally, slides are incubated again with Alexa Fluor 549-labeled streptavidin diluted in 1×ISH buffer for 15 min at 37° C. Slides are washed again in 1×PBD as above before being mounted using VectaShield with DAPI (Vector Laboratories, USA, Cat No H1200) following the manufacturers recommendations. Metaphase preparations are visualized using a Olympus BX53 upright fluorescence microscope and images captured using CellSens software.

### Peptide Nucleic Acid (PNA) In Situ Hybridization

Metaphase cells prepared as described above are spread on glass slides and aged at 65° C. overnight. Slides are washed 2× for 2 min each time at room temperature in 1×PBS before treated with 100 μg/mL RNase A (Sigma, USA, Cat No R4642) for 20 min at 37° C. before being washed 2× 2 min each time at room temperature in 1×PBS followed by 1 was in nuclease free H2O. The slides are dehydrated by passing through a cold (−20° C. ethanol series (70%, 85%, 100%) for 2 min each time and air dried.

Probes (PNA Bio, USA) that detect centromeric, telomeric, or LacO (specific to the hSync) sequences labeled with Alexa-488, Cy3 or Cy5 are reconstituted in deionized formamide to a final concentration of 50 mM and stored at −80° C. Probes are defrosted on ice and probe mixtures are prepared by addition of probes to a final concentration of 500 nM to hybridization buffer (20 mM Tris, pH7.4, 60% deionized formamide, 0.5% blocking reagent (Roche, USA, Cat No 11096176001)). Slides and hybridization mixes are prewarmed separately at 85° C. for 5 minutes. 20 mL of hybridization mix is added to each slide, covered with a coverslip and incubated at 85° C. for 10 minutes. Slides are incubated in the dark at room temperature for 2 hours. Following hybridization, coverslips are removed by briefly washing slides in room temperature wash solution (2×SSC, 0.1% Tween-20) before 2 washes for 10 min each in wash solution at 60° C. Slides are washed a final time in room temperature wash solution for 2 min followed by washes in 2×SSC, 1×SSC and nuclease free H2O before being mounted using VectaShield with DAPI (Vector Laboratories, USA, Cat No H1200) following the manufacturers recommendations. Metaphase preparations are visualized using a Olympus BX53 upright fluorescence microscope and images captured using CellSens software.

### PCR Assays

Genomic DNA: Cells are collected by trypsinization and centrifugation before being resuspended in 50-100 mL of 1×PBS. Genomic DNA is prepared using the QIACube Connect robot (Qiagen, USA) and the QIAamp DNA mini kit (Qiagen, USA, Cat No 51306) following the manufacturers recommendations. DNA concentration and purity is determined using a nanodrop.

Junction PCR assays: PCR amplification reactions to confirm correct integration of therapeutic DNA onto the hSync are carried out using 100-200 mg genomic DNA and OneTaq master mix (New England BioLabs, USA, Cat No M0482S) for 40 cycles using an annealing temperature of 55° C. All DNA fragments were resolved on a 1% agarose gel containing ethidium bromide.

attP: Detection of the attP site is carried out using primers:

CGB0158
(5′ CCTTGCGCTAATGCTCTGTTACAGG 3′),
and
CGB0159
(5′ CAGAGGCAGGGAGTGGGACAAAATTG 3′)

Blastcidin attR and attL: Detection of the Blasticidin attR and attL sites is carried out using primers:

attR-CGB0288
(5′ GCGCTAATGCTCTGTTACAGGT 3′)
and,
CGB0321
(5′ GCAATGGCTTCTGCACAAACA 3′)
attL-CGB0292
(5′ GAGGAAGAGTTCTTGCAGCTCGGT 3′)
and,
CGB0295
(5′ CTGGCGCCAAGCTTCTCTGC 3′)

Zeocin attR and attL: Detection of the Zeocin attR and attL sites is carried out using primers:

attR-CGB0288
(5′ GCGCTAATGCTCTGTTACAGGT 3′)
and,
CGB0567
(5′ ACCACACCGGCGAAGTCGT 3′)
attL-CGB0292
(5′ GAGGAAGAGTTCTTGCAGCTCGGT 3′)
and,
CGB0410
(5′ GGGGCTGCAGGAATTCGATATCAAGCTTC 3′)

Hygromycin attR and attL: Detection of the Hygromycin attR and attL sites is carried out using primers:

attR-CGB0288
(5′ GCGCTAATGCTCTGTTACAGGT 3′)
and,
CGB0297
(5′ CTAGGCCTTTCGCTCAAGTTAGT 3′)
attL-CGB0292
(5′ GAGGAAGAGTTCTTGCAGCTCGGT 3′)
and,
CGB0295
(5′ CTGGCGCCAAGCTTCTCTGC 3′)

PCR assays: PCR amplification reactions to confirm presence of therapeutic DNA sequences on the hSync are carried out using 100-200 mg genomic DNA and OneTaq master mix (New England BioLabs, USA, M0482S) for 40 cycles using an annealing temperature of 55° C. All DNA fragments were resolved on a 1% agarose gel containing ethidium bromide. Primers specific for each therapeutic DNA are designed to confirm presence of coding sequences.

### T-Cell Isolation and Activation

Blood from a healthy donor is collected and mononuclear cells are isolated with a density gradient. The cells are washed and stained with antibodies for CD3. The cells are sustained in t-cell media supplemented with IL-2. The cells are regularly activated with anti-CD3/CD28 beads to induce proliferation.

### QPCR

RNA is extracted from cells or tissues and translated into cDNA. CDNA is mixed with dye and primers and analyzed in a cycler. The gene of interest is normalized to a housekeeping gene and expression can thus be quantified.

### Flow Cytometry

Cells are isolated and washed. Antibodies conjugated with various fluorophores are combined to stain the markers of interest. After staining the cells are run through the analysis instrument where lasers provide photons which are absorbed by the fluorophores and then emitted at different wavelengths. The pattern of absorption and emission is acquired and analyzed to provide a vast amount of data.

### Flow Cytometry Sort

In flow cytometry-based sorting the cells are washed and stained with antibodies conjugated with fluorophores. The difference is in the hardware, in the sorter the pattern of emissions from the fluorophores controls a magnet which opens a valve to let the stained cell trough. The sorted cells are collected and so is the flowthrough.

### Magnetic Bead Sort

In magnetic bead sort antibodies are yet again used to stain surface markers on the cells but in this case the antibodies are conjugated to a magnet. After staining the cells are thoroughly washed and run through a column in a strong magnetic field. The unlabeled cells flow through the magnetic field, but the cells of interest stay. The column is then moved from the magnetic field and the cells are released.

The following examples are put forth so as to provide those of ordinary skill in the art with a complete disclosure and description of how to make and use the present invention and to highlight the features of the invention(s). However, the present disclosure shall in no way be considered to be limited to the particular embodiments described below. These Examples are not intended to limit the scope of what the inventors regard as their invention, nor are they intended to represent or imply that the experiments below are all of or the only experiments performed. It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees centigrade, and pressure is at or near atmospheric.

### EXAMPLES

### Example 1—Production of Synthetic Chromosomes

Plasmid constructions and transfections. Two vectors were constructed to contain the DNA elements desired in the synthetic chromosome. The first vector, pSTV28Hu_rDNA, contained a 10,428 bp Sall fragments encompassing a portion of the human rDNA locus and the chloramphenicol (CAP) selectable marker gene on the pSTV28 plasmid backbone. The Sall rDNA fragment was isolated from HT1080 genomic DNA and cloned into the Sall site of pSTV28 to create pSTV28Hu_rDNA (13,477 bp). The second vector, p15A72LacEF1attPPuro (8656 bp), consists of the EF1alpha promoter driving the puromycin resistance gene and contains the 282 bp attP site between the promoter and puromycin coding sequence. In addition, this vector has a 3436 bp element of the bacteriophage lambda lacO DNA element repeated 48 times in a head-to-head concatemer. In brief, the p15A replication origin was isolated as a 1591 bp Xmnl fragment from pACYC177 and ligated to a 791 bp Hpal/Xmnl fragment from pSP72 and named p15A72. The 2339 bp BamHI/BgIII fragment of p15A72 was then ligated to a 3436 bp BamHI/BgIII fragment containing the lacO repeat created in p15A72 by ligation of BamHI/BgIII lacO multimers into BamHI/BgIII digested p15A72. The resulting vector (p15A7248Lac; 5783 bp) was linearized by PvuII digestion and ligated to a 2872 bp Hpal-PvuII fragment from pEF1alphaattPPuroSV40polyAn containing the puromycin resistance gene driven by the human EF1alpha promoter and creating p15A72LacEF1attPPuro.

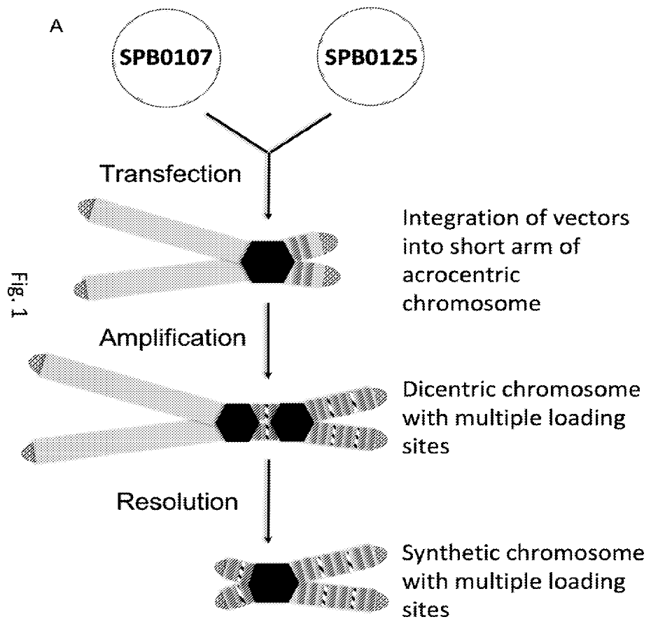

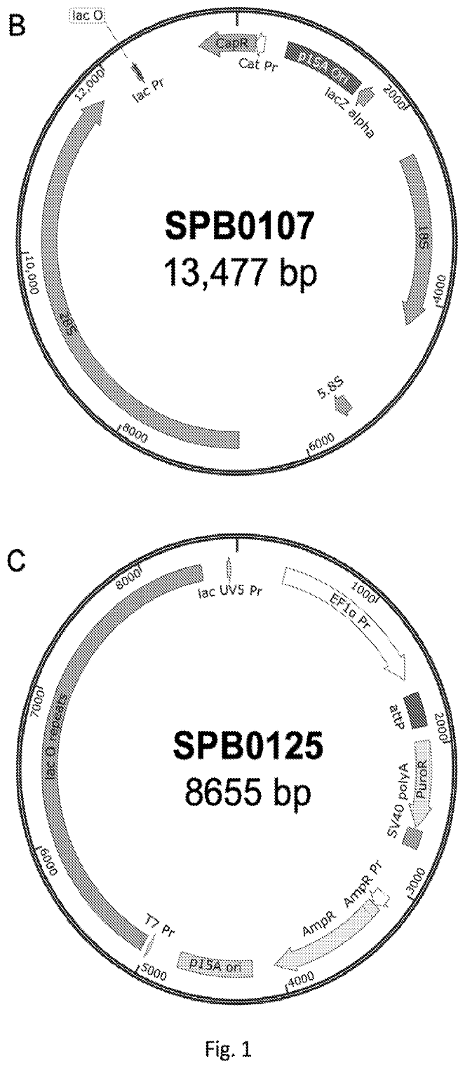

The strategy used to engineer a human synthetic chromosome is outlined in FIG. 1. The pEF1αattPPuro vector was engineered to eliminate CpG sequences in order to diminish the potential host immune response that can be generated towards unmethylated CpG in sequence specific contexts derived from standard bacterial cloning vectors for in vivo applications. In addition to the EF1α promoter, the vector contained the gene conferring puromycin resistance downstream of the promoter, the 282 bp lambda-derived attP sequence, and an array of 48 LacO repeats. The LacO arrays, which are amplified during synthetic chromosome formation, were included to allow in vivo imaging and flow sorting of the chromosome in downstream applications. Linearized pEF1αattPPuro was co-transfected with an excess of a linearized human rDNA-containing vector, thereby targeting integration of both vectors near the pericentric region of acrocentric rDNA containing chromosomes (human chromosomes 13, 14, 15, 21, and 22) and initiating synthetic chromosome formation, into the HT1080 cell line, a near diploid human cell line that exhibits clonal efficiency and genetic stability. The two plasmids, pSTV28Hu_rDNA (SPB0107) and p15A72LacEF1attPPuro (SPB0125), were co-transfected into the HT1080 cell line. Cells were maintained in a 37° C. incubator at 5% C02. HT1080 cells were purchased from ATCC and maintained following the providers recommendations. Cell culture medium was supplemented with 0.5 ug/mL puromycin (InvivoGen, San Diego, CA) to select for the hSync formation.

Drug resistant clones were screened by PCR for the presence of pEF1αattPPuro sequences and a candidate clone, HG3-4, was identified for further analysis. Fluorescent in situ hybridization was carried out to test for the presence of pEF1αattPPuro or LacO sequences on a DNA molecule that also contained elements necessary for chromosome stability, i.e., centromeric and telomeric sequences, respectively. Furthermore, as predicted based on the strategy used to engineer the synthetic chromosome, the pEF1αattPPuro sequences were located on an rDNA containing chromosome (FIG. 2). These results confirm that HG3-4 contains a human derived synthetic chromosome, the hSync. HG3-4 was then subjected to single cell cloning by limited dilution and two independent clones, HG3-4ssc3F and HG3-4ssc4D, were expanded. The hSync was present in both clones indicating mitotic stability over approximately 50 population doublings. In summary, the hSynC retains necessary structural elements to confer chromosome stability (centromeres and telomeres), is derived from an rDNA containing chromosome as would be predicted, contains pEF1αattPPuro sequences and lacO repeats for in vivo imaging and flow sort purification of the synthetic chromosome.

### Example 2—FISH Analysis of hSync Chromosome

Fluorescent in situ hybridization. Metaphase cells were spread on glass slides and aged at 65° C. overnight. Slides were treated with 100 μg/mL RNase A for 20 minutes at 37° C. before being washed twice at room temperature in 1×PBS (phosphate buffered saline). The slides were dehydrated by passing through a room temperature ethanol series (70%, 85%, 100%, in that order) for 2 min each and air dried. Metaphase chromosomes were denatured in 70% formamide/2× saline sodium citrate (SSC) at 70° C. for 2 min before being dehydrated by passing through a second ethanol series at −20° C. as described above and then air dried.

Probe mixtures (100 ng/60 μL of biotinylated attP probes with 100 ng/60 μL of digoxigenin-labeled alpha satellite probe and denatured salmon sperm DNA at a final concentration of 0.4 mg/mL were combined with Hybrisol VII (Cat No. MPRIST13901, Fisher Scientific, USA). The probe mixture was denatured at 75° C. for 10 minutes before being snap cooled on ice. 60 μL of probe mixture was added to a slide then a coverslip was placed on the slide and sealed with rubber cement. Slides were hybridized overnight at 37° C.

To detect the probe signals, coverslips were removed, and slides were washed twice in 2×SSC at 42° C. for 8 minutes each, followed by 2 washes in 50% formamide/2×SSC at 42° C. for 8 minutes each. Slides were briefly rinsed in 1×PBD (18 mM phosphate buffer (30 mM sodium) with 0.01% Triton-X 100, pH 8.0) before being incubated for 1 hour at 37° C. in 1×ISH blocking buffer (Vector Labs). Slides were incubated with Alexa Fluor 488-labeled mouse anti-digoxigenin and Alexa Fluor 549-labeled streptavidin diluted in 1×ISH buffer for 1 hour at 37° C. Slides were washed 3 times for 2 minutes each with agitation in 1×PBD before being incubated for 30 minutes at 37° C. with Alexa Fluor 488-labeled goat anti-mouse IgG and biotinylated-anti-streptavidin diluted in 1× ISH buffer. Slides were washed as above with 1×PBD. Finally, slides were incubated again with Alexa Fluor 549-labeled streptavidin diluted in 1×ISH buffer for 15 min at 37° C. Slides were washed again in 1×PBD as above before being mounted using VectaShield with DAPI following the manufacturers recommendations. Metaphase preparations were visualized using a Nikon Eclipse 80i upright fluorescence microscope and images captured using Nikon Elements software (FIG. 2).

### Example 3—Expression from and Selection of a Gene in Cell Type of Interest

Cells are not always willing to express a gene, it depends on e.g. expression of regulatory elements. Therefore, expression of the wildtype protein is tested in the cell of interest. If the WT protein is difficult to express, then another protein (or version thereof) should be chosen.

### Example 4—Chemokine Receptor Insert in hSync

This chromosome carries multiple chemokine receptor coding genes such as listed in Table 1.

Chemokine are chemotactic peptides that mediate the migration and tissue infiltration of immune cells. Introducing chemokine receptors via hSync into leukocytes can drive their migration into solid tumors and facilitate their effector function. The AR gene helps selecting out transfected cellular clones which have the correct insertion of the cytokine gene cluster on the synthetic chromosome within the cell. attR and attL elements are the recombination products of the site-specific DNA recombination reaction between attP (located on the synthetic chromosome) and attB (located on the loading vector that was constructed to carry one or more chemokine receptors) by bacteriophage lambda integrase directed loading of the chromosome.

### Example 5—Chemokine Receptor Insert in hSync with Supporting Elements

This chromosome carries multiple chemokine receptor coding genes such as listed in Table 1.

The chemokine receptor coding genes are under the regulation of promoters such as truncated or full-size eukaryotic promoters (PKG, EF1) or inducible promoters (TET or other). The chromosome insert may contain markers for selection with affinity (such as truncated CD34), or by fluorescence (such as GFP). The chromosome insert may contain other supporting elements, such as genes that modify T cell activation, proliferation, and tumor-effector function. The chromosome insert may contain genetic insulator elements (such as HS4 or similar) to avoid inappropriate genetic interactions between the cytokine insert and other parts of the chromosome. The AR gene helps selecting out transfected cell clones which have the correct insertion of the cytokine gene cluster. attR and attL elements are sequence products of the site-specific DNA recombination reaction which has built in the cytokine cluster.

Multiple chemokine genes are transcribed by independent promoters or separated by DNA sequences encoding either 2A peptides or IRES elements to form a single transcript containing multiple genes, thereby resulting in nearly equivalent expression levels of the proteins transcribed. 2A self-cleaving peptides that may be employed include but are not limited to: the porcine teschovirus-1 2A (P2A); thosea asigna virus 2A (T2A), equine rhinitis A virus 2A (E2A), foot and mouth disease virus 2A (F2A), cytoplasmic polyhedrosis virus (BmCPV 2A); and flacherie Virus 2A (BmIFV2A) (Table 2). Data indicates that addition of a short 3 amino acid peptide (glycine-serine-glycine) to the N-terminus of the self-cleaving peptide improves self-cleavage (GSG, Table 2). Thus, this example also encompasses slight modifications to improve efficiency of the 2A self-cleaving peptide activity.

TABLE 2

| Name | GSG | 2A Sequence |
|---|---|---|
| P2A | GGAAGCGGA | GCTACTAACTTCAGCCTGCTGAAGCAGGCTGGAGACGTGGAGGA |
| | | GAACCCTGGACCT [SEQ ID NO. 7] |
| T2A | GGAAGCGGA | GAGGGCAGAGGAAGTCTGCTAACATGCGGTGACGTCGAGGAGAA |
| | | TCCTGGACCT [SEQ ID NO. 8] |
| E2A | GGAAGCGGA | CAGTGTACTAATTATGCTCTCTTGAAATTGGCTGGAGATGTTGAGA |
| | | GCAACCCTGGACCT [SEQ ID NO. 9] |
| F2A | GGAAGCGGA | GTGAAACAGACTTTGAATTTTGACCTTCTCAAGTTGGCGGGAGACG |
| | | TGGAGTCCAACCCTGGACCT [SEQ ID NO. 10] |

(See, Kim, et al., PLoS ONE, 6(4), e18556. http://doi.org/10.1371/journal.pone.0018556) Internal ribosomal entry sites (IRES) elements that may be employed include but are not limited to viral and cellular IRES elements. Viral IRES elements are categorized into four types. Type I includes enterovirus (EV, PV, HRV), type II, cardiovirus (EMCV) and aphthovirus (foot-and-mouth disease virus, FMDV), type III, is used for hepatitis A virus (HAV), and the hepatitis C virus (HCV)-like IRES conforms group IV. (see Pacheco and Martinez-Salas, J Biomed Biotechnol. February 2; 2010:458927. doi: 10.1155/2010/458927. PMID: 20150968; and Hellen and Sarnow, Genes Dev., 15(13):1593-612 (2001))

### Example 6—the hSynC-pkgCCR4_tetCCR6 Chromosome

This chromosome carries the human CCR4 and CCR6 gene which supports T cell migration into solid tumors. The CCR4 gene is under the control of PKG promoter and CCR6 is under an inducible promoter such as e.g., tetracycline inducible promoter. The chromosome contains a truncated CD34 gene for affinity purification of hSynC-IL2 chromosome containing transfected cells. The truncated CD34 gene is under the control of EF1α promoter. The chromosome contains HS4 insulator to avoid inappropriate genetic interactions between the chemokine receptor insert and other parts of the chromosome. The BSD gene helps selecting out transfected clones which have the correct insertion of the chemokine receptor gene cluster.

### Example 7—hSynC-pkgCCR4_tetCCR6 T Cells Migrate Towards Chemokine Ligands

To test the function of hSynC-pkgCCR4_tetCCR6 chromosome, hSynC-pkgCCR4_tetCCR6 T cells are placed in transwell plates and their migration towards chemokines is monitored. hSynC-pkgCCR4_tetCCR6 T cells migrate towards CCL22 due to the constitutively expressed CCR4. When tetracycline is added to the T cells, migration towards CCL20 is observed.

Example 8—Activated primary T cells express the CCR4 and CCR6 chemokine receptors after electroporation with plasmids. To test if transgenic CCR4 and CCR6 can be introduced to T cells we have electroporated plasmids with CCR4 or CCR6 genes into T cells from healthy human donor PBMCs. Surface expression and the overall (intracellular) expression of the transgenic chemokine receptors could be detected after 72h with flow cytometry. Cell were stained in PBS 2% FBS with anti-CCR6(PE;FAB195P) or anti-CCR4(PE;BD 561110) for 30 min at 4° C. Since chemokine receptors can be internalized, intracellular expression was measured too. For intracellular staining, cells were fixed and permeabilized (Biolegend kit) before staining with anti-CCR6(PE; FAB195P) or anti-CCR4(PE;BD 561110) 45 min at 4° C. Fluorescent signal was read with Miltenyi Quant 16 (Miltenyi Biotech). We conclude that primary T cells are able to properly express CCR4 and CCR6 chemokine receptors as transgenes.

### Example 9—Cells+hSync=Cellular Medicinal/Therapeutic Product

Autologous tumor-specific T cells have been genetically engineered ex vivo to contain a synthetic chromosome encoding factors that facilitate tumor eradication: the genes C-C chemokine receptor type 6 (CCR6) and Interleukin-2 (IL-2) as therapeutic agents, as well as a gene expressing a truncated version of CD34 as a cell marker, and two independently regulatable (inducible) safety switches.

The compositions and methods described herein provide an autologous cellular cancer immunotherapy that enhances the T cells' inherent ability to eliminate cancer cells by expression of CCR6 and IL-2 from a bioengineered synthetic chromosome. Expression of CCR6 on the cell surface helps direct T cell migration toward tumor metastasis in the liver and improves tumor infiltration and elimination. Upon antigen recognition at the tumor, the T cells express increased amounts of IL-2, thereby facilitating T cell proliferation and cytotoxic activity.

### The Synthetic Chromosome

A synthetic chromosome, hSync, was generated from a human acrocentric chromosome and contains multiple recombination acceptor sites. It was engineered in a similar fashion as other mammalian synthetic chromosome. Briefly, a linearized pEF1αattPPuro vector was co-transfected with an excess of a linearized human rDNA-containing vector into a near diploid human fibrosarcoma cell line. The hSync chromosome was engineered to encode several factors, including: CCR6 to facilitate chemotaxis towards the metastasis site; IL-2 to facilitate T cell activation and cytotoxicity; a truncated version of CD34 (tCD34) allowing isolation of transfected cells; an X-inactivation specific transcript (Xist) lncRNA allowing inactivation of the bioengineered hSync chromosome; and a safety switch in which the antiapoptotic protein BCL2A1 was constitutively expressed at low levels, and pro-apoptotic factors (e.g., BBC3 and/or BCL2L11) were under tetracycline-inducible control, providing to ability to direct apoptosis of the hSync chromosome-bearing cells.

### Tumor Specific T Cells

The chromosome was transfected into T cells that had been harvested from tumor draining lymph nodes and expanded in the presence of a homogenate from the patient's own tumor. In previous work, such autologous T cells have been successfully administered and a therapeutic benefit was observed, but that work was not performed using cells comprising a synthetic chromosome. The presently described hSync was genetically engineered to enhance the tumoricidal activity of these T cells by introducing two therapeutic genes and two independent safety switch systems that can be used to send the synthetic chromosome-bearing transfected cells down an apoptotic pathway or to silence and inactivate the newly introduced chromosome. In addition, the cells express a truncated CD34 protein (tCD34) which was used to identify and isolate transfected cells.
The Cells+Bioengineered Chromosome→Therapeutic Composition

### Qualitative and Quantitative Composition

Dosage of the composition depends on the context of the cancer, the stage of the cancer, the patient's status, and several other factors. In one study, autologous T cells were administered at a median dose of 153×10⁶ cells per patient without any treatment related toxicity. Consequently, the dose of the cell+synthetic chromosome therapeutic composition can range from 10⁶-10⁸ viable T cells, similar to the dose range used in Chimeric antigen receptor T cell therapies. In some embodiments, if the synthetic chromosome carries multiple copies of a particular therapeutic agent, a smaller number of therapeutic cells may be used. In some embodiments, the dose can comprise as few as 10⁴ or as many as 10¹⁰ viable cells.

### Mode of Administration

The (cell+synthetic chromosome) therapeutic composition is intravenously infused according to the guidelines of the hospital in which the treatment will take place, similarly to what has previously been described. Alternative methods of delivery may include intramuscular, intracranial, direct injection into disease tissue (e.g., injection into tumor beds), intraocular, subcutaneous injection, as well as encapsulated delivery and in vivo delivery/transfection.

### Pharmaceutical Form

The transfected patient T cells were harvested, washed with saline solution and then resuspended in saline solution supplemented with 1% human serum albumin. The finished product can be provided in the form of a cell suspension for infusion.

### Mechanism of Action/Proposed Use*

Immunotherapies have revolutionized the treatment of cancer, but limitations remain and there is still room for improvements. A sentinel-node derived T cell therapy was developed for bladder cancer and colon cancer. The sentinel node is defined as the first tumor-draining lymph node along the direct drainage route from the tumor, and in case of dissemination, it is considered to be the first site of metastasis. The sentinel node is enriched for tumor-reactive T cells. In brief, this treatment modality is based upon surgically harvesting tumor-draining lymph nodes followed by in vitro expansion of the T cells using tumor extracts, and subsequent reinfusion of these autologous tumor-specific T lymphocytes. Previous clinical studies have demonstrated a significantly increased 24-month survival rate after using this treatment. Importantly, no significant side-effects were observed after intravenous administration of expanded sentinel node T cells.

While sentinel-node derived T cell therapy is promising, the majority of patients do not respond, as is the case for all cancer immunotherapies. Thus, the composition and methods described herein provide for enhancement of the tumoricidal effect of these T cells by equipping them with synthetic chromosomes that encode the IL-2 and CCR6 proteins to increase the maintenance, activation and homing of the T cells, as well as safety switches that can be used to carefully control the fate of the synthetic chromosome and chromosome transfected cells.

IL-2 was the first cytokine to be discovered and was initially known as “T cell growth factor”. IL-2 is predominantly produced by antigen-simulated CD4⁺ T cells, and acts in an autocrine or paracrine manner. IL-2 production can lead to autocrine stimulation as well as effector T cell survival. IL-2 is an important factor for the maintenance of CD4⁺ regulatory T cells and plays a critical role in the differentiation of CD4⁺ T cells. It can promote CD8⁺ T-cell and NK cell cytotoxicity activity and modulate T-cell differentiation programs in response to antigen, promoting naive CD4⁺ T cell differentiation into T helper-1 (Th1) and T helper-2 (Th2) cells. Recombinant IL-2, as a monotherapy, was approved for metastatic renal cell carcinoma in 1992 and in 1998 it was approved for metastatic melanoma by the FDA. Although IL-2 has been demonstrated to be capable of mediating tumor regression, it is insufficient to improve patients' survival due to its dual functional properties on T cells and severe adverse effect when presented in high dose. In the presently disclosed compositions and methods, expression of IL-2 is carefully controlled, and IL-2 is present at only slightly higher than normal levels (e.g., between 1.5- and 10-fold higher than average levels observed in healthy patients) upon T cell recognition of tumor antigens. This low-level expression of IL-2 facilitates anti-tumor immune T cell responses without provoking adverse side-effects. The previously observed side effects occurred when recombinant IL-2 was supplied at levels several orders of magnitude higher than normal physiological levels.

The G-protein coupled receptor CCR6 is naturally expressed in lymphatic cells. The fact that the CCR6 receptor binds specifically to one ligand, Chemokine (C-C motif) ligand 20 (CCL20), makes it particularly useful to the present compositions and methods. The CCL20-CCR6 axis is involved in tissue inflammation and homeostasis but this natural axis is often hijacked in cancer progression. The liver is a common site for metastases from many cancer types, most commonly colorectal cancer. Colorectal cancer cells express both CCL20 and CCR6. Thus, an autocrine and paracrine loop leads to increased proliferation and migration of the cancer cells. Increased CCR6 expression in colorectal tumors is strongly associated with metastasis and poor prognosis for the patient. Animal studies where CCR6 is over expressed in CAR-T cells show that the cells have an increased migration to the tumor site and also infiltrate and clear the tumor when reaching the site. By inclusion of CCR6 in the cell+synthetic chromosome therapeutic composition, the tumor's weapons are turned against itself. CCR6 helps the T cells to migrate towards the tumor site and infiltrate the tumor.

In sum, the mechanism of action is the combination of engineered tumor-specific T cells that express IL-2 to amplify anti-tumor responses and CCR6 to facilitate chemotaxis to the tumor.

### Properties

This treatment modality consists of tumor-specific T cells that express higher than normal levels of IL-2 and traffic towards CCL20 expression sites in the body, such as a colon cancer liver metastasis.

### Current Manufacturing Strategy

hSync Production

The human synthetic chromosome, hSync, was engineered as follows: In brief, an EF1αattPPuro cassette containing an EF1α promoter, a 282 bp lambda-derived attP sequence, an array of 48 LacO repeats and the gene conferring puromycin resistance was co-transfected with an excess of a linearized human rDNA-containing vector into the human HT1080 fibrosarcoma cell line. The rDNA facilitates integration of both vectors near the pericentric region of human acrocentric chromosomes and initiates synthetic chromosome formation. The pEF1αattPPuro vector was engineered to eliminate CpG sequences in order to diminish any potential host immune response that can be generated towards unmethylated CpG motifs. Drug resistant clones were evaluated by PCR targeting pEF1αattPPuro sequences and a candidate clone, HG3-4, was selected for subsequent analysis and evaluation. Presence of the synthetic chromosome was assessed by fluorescent in situ hybridization (FISH) directed towards pEF1αattPPuro or LacO sequences, centromeric and telomeric sequences. Single cell cloning and expansion of two independent clones, HG3-4ssc3F8 and HG3-4ssc4D10, demonstrated hSync mitotic stability over approximately 50 population doublings in the HT1080 cell line. The hSync was then transferred into Chinese Hamster Ovary CHO-K1 cells, an exemplary cell line for eventual bulk production of chromosomes. FISH and PCR was used to confirm the chromosomal integrity and the presence of human specific alpha satellite sequences and the pEF1αattPPuro attP sequences.

The hSync, was easily isolated and transferred to a recipient cell line while retaining all bioengineered and native structural elements and stably maintained in the recipient cell line for well over 50 population doublings.

### Constructs

The hSync synthetic chromosome specific to the composition of this Example encodes CCR6, IL-2, tCD34 and two independent safety systems. These elements are introduced into the hSync using a mutant lambda integrase (ACE integrase) and the attP/attB recombination sites. Successful recombination resulted in the drug resistance gene being integrated downstream of the EF1α promoter contained on the hSync, thereby conferring drug resistance on clones that incorporated the genes of interest onto the hSync. In addition to the attB donor recombination site and drug resistance marker, all constructs contained tCD34 expressed from the PGK1 promoter to allow quantitative tracking of cells containing the hSync. The extracellular domain of CD34 was shortened by alterations to exons 1 and 2. Additionally, modifications to exons 7 and 8 ensure that no intracellular signaling takes place in the transfected cells.

The first safety switch construct, in addition to the common elements, contains one or both of two pro-apoptotic genes, BBC3 and BCL2L11, under the control of a tetracycline responsive promoter, which allows the expression to be tightly controlled. The safety switch construct also contains BCL2A1, an antiapoptotic gene constitutively expressed from the PGK1 promoter.

The second, independent safety switch system, based on X chromosome inactivation, can be achieved by expression of Xist lncRNA under control of a regulatable promoter. In this Example, a construct was designed to allow inactivation of the hSync by expression of the Xist lncRNA element under the control of a Tamoxifen inducible promoter. In some embodiments, an estrogen receptor-based transactivation system “XVER” can be used to inactivate hSync.

In some embodiments, eHAP cells are used. In other embodiments, a safety switch is envisioned and could be designed to be regulated by a small molecule, antibiotic, or other therapeutic compound, such that the hSync chromosome can be inactivated by inducing expression of the Xist lncRNA upon administration of the small molecule, antibiotic, or other therapeutic compound.

Tamoxifen, a selective estrogen receptor modulator (SERM), is one example of a compound that can be employed to bind and regulate a promoter; in this embodiment, expression of the chromosome-silencing Xist lncRNA (or a therapeutic agent, or other component encoded on the hSync) was regulated using a Tamoxifen-inducible promoter. Tamoxifen has mixed estrogenic and antiestrogenic activity, with its profile of effects differing by tissue (i.e., it has predominantly antiestrogenic effects in the breasts but predominantly estrogenic effects in the uterus and liver).

All genetic elements were initially tested separately by transfection of plasmid constructs into cell lines or primary cells, including the CHO-K1 (ATCC Cat #CCL-61), MOLT4 (ATCC Cat #CRL-1582), Jurkat (ATCC Cat #TIB-152) and HT1080 (ATCC Cat #CCL-121) cell lines. Experimental data from transfected Jurkat T cells and primary CD4⁺ T cells indicate that the tCD34 marker can be used to sort cells both by flow cytometry, or magnetic beads can also be used. In some embodiments, such as when cells are used that may be more difficult to transfect, magnetic beads may be a preferable way to sort transfected cells. After investigating different combinations of pro- and antiapoptotic genes, it was observed that having both BBC3 and BCL2L11 under a tetracycline induced promoter in combination with a low continuous expression of BCL2A1 was beneficial.

All final constructs were sequence-verified prior to loading onto the hSync. Following transfection and selection, drug resistant colonies were ring-cloned or flow sort purified and then expanded. Genomic DNA, isolated from candidate clones using the Qiagen QIAcube Connect following the manufacturers' recommendations, was used as template in PCR reactions to confirm that the construct has recombined onto the hSync. Primers for the PCR reaction that confirm correct loading construct recombination onto the hSync were designed based on the loading vector used (i.e., which drug resistance gene was present in the targeting vector) and on the sequence of the hSync. Further characterization of newly engineered clones containing the genes of interest was accomplished by PCR of each open reading frame or exon of every expression cassette loaded onto the hSync. Clones in which the construct of interest was confirmed to have been incorporated correctly onto the hSync were subjected to functional assays (e.g., tetracycline induced apoptosis in the case of the Safety Switch).

### Transfection Methods

During chromosome bioengineering, mitotically active cells were transfected with standard lipid-based transfection reagents following the manufacturer's recommended conditions. For each cell line, transfection conditions (e.g., lipid:DNA ratio) were optimized. Constructs to be loaded onto the chromosome were co-transfected at a 1:1 molecule ratio with an engineered bacteriophage lambda mutant integrase that drives unidirectional recombination in mammalian cells. Twenty-four hours post-transfection the cells were placed on drug selection.

Transfer of engineered, flow sort purified chromosomes to recipient cell lines was performed utilizing commercially available chemical transfection methods. However, T cells are small and their cytoplastic space has a limited capacity for the type of endocytosis needed in chemical transfections. A range of chemical transfection methods can be used, as well as various methods of mechanical transfection methods (e.g., microinjection and nano straws).

### Patient Screening and Cell Harvest

Patient inclusion and exclusion criteria include cancer progression, expected survival, tumor manifestation, blue-dye allergy, history of autoimmune diseases as well as ongoing and previous treatments and medications. Patients were also screened for communicable diseases such as hepatitis B- and C virus, human immunodeficiency virus and syphilis, according to the current regulations for the donation of cells and tissues.

Once cleared, the patient undergoes surgery and T cells are obtained from sentinel lymph nodes (SLNs) as described previously. SLNs are intraoperatively identified by injection of patent blue under the serosa that surrounds the primary tumor. When visible, the SLN is excised and subjected to analysis by flow cytometry and ex vivo expansion.

### Quality Control

An extensive list of release criteria and quality control procedures including in-process controls, product integrity and quality testing, safety testing and efficacy testing have been described (Yonghong et al., 2019, “Quality Control and Nonclinical Research on CAR-T Cell Products: General Principles and Key Issues.” Engineering, 5:122-131). Tests may include:
Chromosome integrity and genomic stability (e.g. FISH, Flow-FISH, CASFISH and/or PCR)
Cell count and viability
T cell phenotype
Sterility testing (e.g., fungal, anaerobic and aerobic bacterial contamination, mycoplasma and endotoxin measurements)
Potency assays
Safety switch testing

### Outline of Non-Clinical Development

### Mouse Models

The largest risk with introducing manipulated T-cells is adverse immunological events. To address this issue, a safety mechanism was included in engineered therapeutic cell+synthetic chromosome composition that will eliminate all cells containing the hSync that have been introduced to the body. This safety switch is based on tetracycline-inducible expression of pro-apoptotic factors such as BBC3 or BCL2L11 using the Tet-on system. Tetracycline is a widely used antibiotic with few and manageable side effects. As the Tet-on system displays a low level of promoter leakiness, the antiapoptotic protein BCL2A1 is introduced at low constitutive expression levels, which facilitates cell survival. Thus, all cells in the therapeutic cell+synthetic chromosome composition have a dual-action safety switch that normally facilitates cell survival but induces cell death when triggered by administration of Tetracycline. To test this system, the Jurkat T cell line was transfected with an hSync that encodes the safety switch. These Jurkat cells were transferred into immunodeficient mice together with untransfected cells in a 1:1 ratio, followed by administration of Tetracycline intraperitoneally 1-, 2- and 4-weeks post injection. Flow cytometry was then used at 24-, 48- and 72-hours post-Tetracycline administration to determine the relative ratio of transfected and untransfected Jurkat T cells and consequently the efficiency of the safety switch.

One roadblock to wide implementation of gene-therapy is the inability to turn off gene expression once therapy is completed. Xist, a long non-coding RNA that normally facilitates X chromosome inactivation in females acts in cis to induce heterochromatinization of the chromosome from which it is expressed. A whole chromosome off switch was created based on Xist, in order to inactivate expression of the therapeutic agent(s) delivered with composition. To accomplish this, the therapeutic cell+synthetic chromosome composition was engineered such that the Xist lncRNA was expressed under regulatable control of a Tamoxifen-inducible promoter, which allows precise control of Xist lncRNA expression from the synthetic chromosome. Administration of tamoxifen results in silencing of the synthetic chromosome, while allowing the tumor-specific T cells to persist. The Xist element has also been tested in vivo using the Jurkat cell line. In brief, hSync transfected Jurkat T cells were transferred into immunodeficient mice followed by administration of tamoxifen and analysis of the degree of hSync inactivation.

### Cell Activity Assays

The mechanisms of action of IL-2 and CCR6 were tested in vitro. In brief, the synthetic chromosome-transfected primary T cells were tested using the classical Boyden Chamber Assay to determine their capability to migrate towards a gradient of CCL20, the unique ligand for CCR6. In regard to IL-2, the synthetic chromosome-transfected primary T cells were assayed for their ability to produce IL-2 using ELISA and PCR. In addition, the proliferation of these cells was monitored and compared to untransfected cells using CFSE dilution assays. Finally, the cytotoxic activity of the cell+synthetic chromosome composition transfected CD8⁺ T cells was determined.

### Example 10—Testing the Efficacy of Tamoxifen-Inducible Silencing by the Xist lncRNA

The following Example is illustrative of how inducible expression of Xist introduced as a transgene can be used to drive inactivation of target sequences on the synthetic chromosome in synthetic chromosome-bearing cells. For example, after induction of the Xist lncRNA by Tamoxifen using the system described above, the inactivation of expression of a DsRed-DR fluorescent protein marker (RFP) can be assessed in the transfected cells, as compared to the fluorescence levels of control cells (such as cells carrying the synthetic chromosome but not induced).

A synthetic chromosome has been engineered to contain RFP, for example, and DNA sequences to be loaded onto the synthetic chromosome were first transferred to the pAPP chromosome loading vector. Four vectors containing green fluorescent protein (GFP) gene fused to the blasticidin resistance gene (BSR) have been engineered for this use. In some embodiments, a vector may contain a pair of modified loxP sites flanking the GFP-BSR allowing it to be recycled for repeated synthetic chromosome loadings. Once the first DNA sequence is loaded and the chromosome analyzed, cells are transfected with Cre recombinase, resulting in excision of the GFP-BSR making the clone amenable to loading of a second DNA sequence with blasticidin selection. In this way, the GFP-BSR cassettes can be recycled. Following Cre excision, cells were sorted to isolate those that no longer express GFP. Correct excision of the GFP-BSR cassette is confirmed by PCR prior to loading a subsequent DNA sequence. At each step, the engineered synthetic chromosomes are assessed for correct integration using PCR-based assays that confirm appropriate targeted integration onto the platform synthetic chromosome. The presences of resulting attB×attP recombination products (attR and attL junctions) are confirmed by PCR.

The pAPP chromosome loading vector was engineered to contain the DsRed-DR coding sequence (Clontech, Mountain View, CA), which has a destabilized variant of Discosoma sp. derived red fluorescent protein with a short half-life, under regulation of the CMV promoter. DsRed-DR was loaded onto the synthetic chromosome and single cell clones with bright fluorescence were isolated by FACS. The tetracycline-controlled transactivator, tTA, was then loaded onto the synthetic chromosome in clones with highest DsRed-DR expression. In some embodiments, clones with undetectable background expression and high levels of expression in the absence of the tetracycline analog doxycycline (Dox) were identified using a luciferase reporter construct under control of the tetracycline responsive element (TRE). In other embodiments, the system can be designed to be “TET ON”, i.e. expression is undetectable without doxycycline, and high level expression can be induced in the presence of doxycycline.

The Xist cDNA (Origene) was cloned into the pTRE-Tight tetracycline response vector to minimize background expression. The TRE-Tight-Xist construct was transferred to the pAPP loading vector as described above and subsequently loaded on the synthetic chromosome. In this instance, DG44 cells were cultured in the presence of doxycycline to ensure the Xist cDNA is not expressed prematurely. Once clones were selected, the DG44 cells were transferred to medium either with or without doxycycline and mRNA was isolated every 24 hours for 5 days. Xist expression levels were assessed by real time PCR. Clones with tight, inducible expression of Xist were used for downstream experiments.

Xist expression in the differentiated DG44 cells did not result in inactivation of DsRed DR expression; however, the cells were assessed microscopically for red fluorescence. If red fluorescence was quenched in DG44 in the absence of doxycycline, real time PCR is used for confirmation that this is due to silenced expression. Additionally, it was determined that the synthetic chromosome had become heterochromatinized.

Loss of DsRed-DR fluorescence was confirmed to be due to silenced expression using quantitative real time PCR to assess mRNA levels. Taqman assays (Applied Biosystems, Foster City, CA) were used to detect expression of the Xist long non-coding RNA. A custom Taqman assay was designed for detection of DsRed-DR. Expression levels of DsRed-DR were normalized to the endogenous control GAPDH expression levels, expressed from host cell chromosomes. This also acted as a control to demonstrate that silencing is limited to genes on the synthetic chromosome. DsRed-DR expression levels were correlated with the frequency of red fluorescent cells in the population. Expression of Xist (-Doxycycline group) was correlated with fewer red fluorescent cells, which in turn was correlated with decreased DsRed-DR mRNA levels compared to cells cultured in the presence of doxycycline.

### Assay Heterochromatinization Following Xist Expression

Two markers of heterochromatinization were quantified to assess the levels of condensation following Xist expression: heterochromatin protein 1 alpha (HP1α), a marker of constitutive heterochromatin, and histone H3 tri-methylated on lysine 27 (triMe-H3K27), a marker of facultative heterochromatin found on the inactive X chromosome. Metaphase spreads were prepared by cytospin following hypotonic treatment in 0.07M KCl for 10 minutes at room temperature. Following fixation in 4% paraformaldehyde, cells were blocked in 3% BSA for 30 minutes. Synthetic chromosomes were incubated with a mouse monoclonal antibody to HP1α (ab151185; Abcam) or a rabbit polyclonal antibody to triMe-H3K27 (EpiGenTek) prior to incubation with appropriate fluorochrome conjugated secondary antibodies (Jackson ImmunoResearch). Synthetic chromosomes were then stained with DAPI and imaged. The synthetic chromosomes were identified by FISH with a probe directed against the attPPuro sequence. An increase in triMe-H3K27 on the synthetic chromosomes following Xist expression was observed, while HP1α levels remained unchanged at pericentromeric regions, acting as a normalization control. In addition, levels of histone H4 acetylation on the synthetic chromosomes were quantified, which follows H3K27 tri-methylation during X inactivation, during the time course of each experiment.

As an alternative approach, the EpiQuik Chromatin Accessibility Assay Kit (EpiGenTek) can be used to assess chromatin accessibility. This kit combines nuclease sensitivity with a subsequent real time PCR assay to measure the chromatin structure of specific regions. DNA prepared from cells grown in the presence and absence of doxycycline are either mock treated or treated with nuclease. Real time PCR using primers for the attB sites along the synthetic chromosome as well as ones designed for the TRE controlling DsRed-DR expression can be used to amplify the selected regions. If chromatin is condensed (heterochromatinized) the DNA is inaccessible to the nuclease and the target region is amplified. If the chromatin is in an open configuration, it is accessible to the nuclease and amplification of the target region is decreased or undetectable. Primers to control constitutively expressed and silenced regions are provided.

### Example 11—Expression of Chimeric Antigen Receptors (CARs) or Antibody Fragments, e.g., Multiple scFv Fragments-on a Synthetic Chromosome Using Two Separate Inducible Promoter Systems

Clinical experience shows that multi-targeted approaches to cancer therapy and infectious disease are generally superior to single agent treatments. Based on their plasticity and robustness, mesenchymal stem cells (MSC) have been implicated as a novel therapeutic modality for the treatment of cancer and infectious disease. As such, bioengineered MSCs, or other additional stem cell populations, hold exceptional utility as novel weapons against cancer and infectious disease for which effective therapies are lacking. Furthermore, the localized delivery of therapeutic factors delivered via stem cell-based therapy may circumvent pharmacological limitations associated with systemic delivery of particularly toxic agents. The combination of synthetic chromosomes engineered to deliver multiple and regulable therapeutic factors has enormous potential as a therapeutic approach that can be tailored to target different disease states.

Single-chain fragment variable (scFv) proteins are attractive therapeutic agents for targeted delivery of cytostatic/cytotoxic bioreagents. scFvs are small antigen-binding proteins made up of antibody VH and VL domains that can exquisitely target and penetrate tumor beds or target infectious diseases agents. The small size of scFvs makes them amenable to fusing with cytotoxic proteins for immunotoxin-based gene therapy. The regulable production of multiple scFvs from the synthetic platform chromosome both in vitro and in vivo is demonstrated utilizing a number of select tumor marker scFvs. For example, commercially available scFv DNA clones targeting Her2 (ErbB2); basigen; c-kit; and carcinoembryonic antigen (CEA) may be useful in some embodiments of the present disclosure (Source BioScience, Inc., Addgene). The scFv encoding DNA regions from commercially available constructs can be amplified by PCR and N-terminal fusions made with luciferase as a reporter (New England Biolabs, Inc). In some embodiments, a fusion construct employs the secreted Gaussia or Cypridina luciferase reporter genes. The utilization of these two ultrasensitive secreted luciferase reporters permits monitoring of expression in a dual assay format, as each luciferase utilizes a unique substrate (i.e. the detection of one luciferase can be measured without any cross-reactivity from the presence of the other in a given sample).

In some embodiments, the expression cassette can include a fusion protein cassette. In some embodiments, the expression cassette is flanked by lox sites to permit recycling of the selectable marker.

In some embodiments, expression cassettes are placed under the control of the TET ON promoter (TetP). For multiregulatable expression, the Cumate Switch ON system (system commercially available from System Biosciences Inc.) also can be utilized. Similar to the TET ON system, the Cumate Switch On system works by the binding of the Cym repressor (cymR; originally derived Pseudomonas) to cumate operator sites downstream of the CMV5 promoter to block transcription.

In the presence of cumate, the repression is relieved allowing for transcription. The Cumate Switch ON system has been used extensively in in vitro applications and is comparable to performance with the TET-ON system. scFv3 and scFv4 CLuc fusions are placed under the control of the Cumate Switch On promoter. Polyadenylation signals and strong transcription termination sequences are placed downstream of all scFv expression cassettes.

In some embodiments, a delivery vector is used, and the delivery vector contains the attB recombination sequence upstream of a GFP-fusion protein cassette. In some embodiments, the expression cassette can be an scFv expression cassette cloned in tandem onto a BAC derived pAPP delivery vector with each expression cassette separated by matrix attachment regions to promote optimal expression and to block transcriptional read through from one cassette to another.

Blasticidin resistance (BSR) is selectable in bacteria due to the presence of the bacterial E2CK promoter within an engineered intron of the GFP-BSR fusion. One exemplary vector, the scFv multi-regulable expression BAC, contains all of the scFV expression cassettes and is approximately 21 Kbp in size (pBLoVeL-TSS_DualExp_scFv). In some embodiments, useful elements are present in the constructs, including: sopA, sopB, and sopC=plasmid partitioning proteins; SV40pAn, B-Globin poly An=poly A; TTS=transcription termination signal; attB=site specific recombination site; lox=site specific recombination site; eGFP=fluorescent protein; Bsr=blasticidin resistance gene; repE=replication initiation site; Ori2=origin of replication; CmR=chloramphenicol resistance gene; polyAn=poly A; Her 2 scFv, c-Kit scFv, CEA scFv=single-chain fragment variable (scFv) proteins; Tet-responsive promoter or CMV+CuO promoter=inducible promoters)).

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims. In the claims that follow, unless the term “means” is used, none of the features or elements recited therein should be construed as means-plus-function limitations pursuant to 35 U.S.C. § 112, ¶6. All references cited herein are hereby incorporated by reference into the detailed description for all purposes.

While various specific embodiments have been illustrated and described, it will be appreciated that various changes can be made without departing from the spirit and scope of the invention(s). Therefore, it is to be understood that the disclosure is not to be limited to the specific embodiments disclosed herein, as such are presented by way of example. Although specific terms are employed herein, they are used in a generic and descriptive sense only and not for purposes of limitation.

All literature and similar materials cited in this application, including, but not limited to, patents, patent applications, articles, books, treatises, internet web pages and other publications cited in the present disclosure, regardless of the format of such literature and similar materials, are expressly incorporated by reference in their entirety for any purpose to the same extent as if each were individually indicated to be incorporated by reference. In the event that one or more of the incorporated literature and similar materials differs from or contradicts the present disclosure, including, but not limited to defined terms, term usage, described techniques, or the like, the present disclosure controls.

Extracts from the Priority Document Covering Aspects of the Invention

Several embodiments of the present disclosure are described in detail hereinafter. These embodiments may take many different forms and should not be construed as limited to those embodiments explicitly set forth herein. Rather, these embodiments are provided so that this disclosure will be thorough and complete, and will fully convey the scope of the present disclosure to those skilled in the art.

Specific embodiments disclosed are:
1. A therapeutic composition comprising:
eukaryotic cells bearing a synthetic chromosome that autonomously replicates and is stably maintained over the course of at least 10 cell divisions, said synthetic chromosome comprising:
an rDNA-amplified centromere region;
a marker allowing for isolation of synthetic chromosome-bearing cells; at least one encoded therapeutic; and
at least one safety switch.
2. The composition of embodiment 1, wherein the eukaryotic cells are autologous human T cells for administration to a patient having a solid tumor cancer.
3. The composition of embodiment 1, wherein the therapeutic facilitates chemotaxis.
4. The composition of embodiment 3, wherein the therapeutic is a CCR6 gene.
5. The composition of embodiment 1, wherein the therapeutic facilitates T cell activation and cytotoxicity.
6. The composition of embodiment 5, wherein the therapeutic is an IL-2 gene.
7. The composition of embodiment 1, wherein the marker allowing for isolation of synthetic chromosome-bearing cells is a truncated version of CD34 (tCD34).
8. The composition of embodiment 1, wherein the synthetic chromosome comprises the CCR6 gene, the IL-2 gene and a gene encoding tCD34.
9. The composition of embodiment 1, wherein the at least one safety switch comprises at least one of the group consisting of:
a whole-synthetic-chromosome-inactivation switch; and
a synthetic chromosome-bearing therapeutic cell-off switch.
10. The composition of embodiment 9, wherein the whole-synthetic-chromosome Inactivation switch comprises at least one Xic gene product selected from the group consisting of Xist and Tsix.
11. The composition of embodiment 9, wherein the synthetic chromosome-bearing therapeutic cell-off switch provokes apoptosis of the synthetic chromosome-bearing-cells.
12. The composition of embodiment 11, wherein the synthetic chromosome-bearing therapeutic cell-off switch comprises at least one pro-apoptotic factor selected from BBC3 and BCL2L11, and optionally comprises an antiapoptotic counterbalancing component, BCL2A1.
13. The composition of embodiment 12, wherein BCL2A1 is present, and is constitutively expressed at low levels.
14. The composition of embodiment 11, wherein both BBC3 and BCL2L11 are present and under control of at least one regulatable promoter.
15. The composition of embodiment 1, wherein expression of at least one of:
(i) the therapeutic(s); and
(ii) the switch from the synthetic chromosome is coordinately regulated by a complex biological circuit.
16. The composition of embodiment 1, further comprising pharmaceutically acceptable components for intravenous delivery.
17. The composition of embodiment 15, wherein expression is induced or repressed by:
(i) an agent selected from the group consisting of tamoxifen, tetracycline, cumate, or any derivative thereof;
(ii) an endogenous regulatory system; or
(iii) a synthetic promoter utilizing TALENS and CRISPR technology.
18. A eukaryotic cell comprising a synthetic chromosome that autonomously replicates and is stably maintained over the course of at least 10 cell divisions, said synthetic chromosome comprising:
an rDNA-amplified centromere region;
a marker allowing for isolation of synthetic chromosome-bearing cells; at least one encoded therapeutic; and
at least one safety switch.
19. The cell of embodiment 18, wherein the cell is an autologous human T cell.
20. A method for generating a therapeutic autologous T cell composition comprising a synthetic chromosome, said method comprising:
Isolating a tumor-draining lymph node from a subject having cancer;
harvesting educated T cells from the lymph node;
expanding the educated T cells ex vivo in the presence of tumor homogenate from the subject;
transfecting the expanded educated T cells with a stable synthetic chromosome comprising:
(i) a marker allowing for isolation of synthetic chromosome-bearing cells;
(ii) at least one safety switch; and (iii) a cassette for regulatable expression of at least one therapeutic agent;
isolating the marker-bearing transfected T cells comprising the stable synthetic chromosome;
confirming regulatable expression of the therapeutic agent; and
combining the transfected, marker-bearing T cells confirmed to have
regulatable expression of the therapeutic agent(s) with biocompatible ingredients to form a cell suspension for infusion into the subject having cancer.
21. A method for treating a solid tumor cancer comprising:
intravenously delivering the therapeutic autologous T cell composition comprising the synthetic chromosome of c embodiment 20 to the subject having a solid tumor cancer.
22. The method of embodiment 21, wherein the cancer is selected from colon cancer, urinary bladder cancer.

### Visualization, Isolation, and Transfer to Recipient Immune Cells

The production and loading of the synthetic platform chromosomes of the present invention can be monitored by various methods. Lindenbaum, M., Perkins, E., et al., Nucleic Acid Research, 32(21):e172 (2004) describe the production of a mammalian satellite DNA based Artificial Chromosome Expression (ACE) System. In this system, conventional single color and two-color FISH analysis and high-resolution FISH were carried out using PCR generated probes or nick-translated probes. For detection of telomere sequences, mitotic spreads were hybridized with a commercially obtained peptide nucleic acid probe. Microscopy was performed using fluorescent microscopy. Alternatively, Perkins and Greene, PCT/US16/17179 filed 9 Feb. 2016, describes compositions and methods to allow one to monitor formation of synthetic chromosomes in real-time via standardized fluorescent technology using two labeled tags: one labeled tag specific to endogenous chromosomes in the cell line used to produce the synthetic platform chromosomes, and one differently-labeled tag specific to a sequence on the synthetic chromosome that is to be produced.

Isolation and transfer of synthetic chromosomes typically involves utilizing microcell mediated cell transfer (MMCT) technology or dye-dependent, chromosome staining with subsequent flow cytometric-based sorting. In the MMCT technique, donor cells are chemically induced to multinucleate their chromosomes with subsequent packaging into microcells and eventual fusion into recipient cells. Establishing that the synthetic chromosomes have been transferred to recipient cells is carried out with drug selection and intact delivery of the transferred chromosome confirmed by FISH. Alternatively, flow cytometric-based transfer can be used. For flow cytometric-based transfer, mitotically arrested chromosomes are isolated and stained with DNA specific dyes and flow sorted based on size and differential dye staining. The flow-sorted chromosomes are then delivered into recipient cells via standard DNA transfection technology, and delivery of intact chromosomes is determined by FISH or Flow-FISH. In yet another alternative, in addition to the visualization and monitoring of synthetic chromosome production, the synthetic chromosome tags can be used to isolate the synthetic chromosomes from the synthetic chromosome production cells via flow cytometry, as well as to monitor the transfer of the synthetic chromosomes into recipient cells.

### Transforming Mammalian Target Cells

To date, isolation and transfer of artificial chromosomes has involved utilizing microcell mediated cell transfer (MMCT) technology or dye-dependent chromosome staining with subsequent flow cytometric-based sorting. In the MMCT technique, donor cells are chemically induced to multinucleate their chromosomes with subsequent packaging into microcells and eventual fusion into recipient cells. The establishment of transferred chromosomes in the recipient cells is carried out with drug selection and intact delivery of the transferred chromosome confirmed by FISH. For flow cytometric-based transfer, mitotically arrested chromosomes are isolated and stained with DNA specific dyes or DNA sequence specific probes or DNA sequence-specific engineered proteins such as native repressors (e.g. lac repressor), TALON engineered proteins, CRISPR-Cas9 derivatives, and engineered Zn finger nucleases. Using these methods, the synthetic chromosomes can be simply flow-sorted based on size and differential dye staining, and the flow-sorted chromosomes are then delivered into recipient cells via standard DNA transfection technology, and delivery of intact chromosomes is determined by FISH or Flow-FISH.

Peptide nucleic acids (PNAs) are an artificially synthesized polymer similar to DNA or RNA. Commercially available fluorescently labeled PNAs can be used to visualize the hSyncs of the present disclosure. For example, New England Biolabs (NEB®) offers a selection of fluorescent labels (substrates) for SNAP- and CLIP-tag fusion proteins. SNAP tag® substrates consist of a fluorophore conjugated to guanine or chloropyrimidine leaving groups via a benzyl linker, while CLIP-tag™ substrates consist of a fluorophore conjugated to a cytosine leaving group via a benzyl linker. These substrates will label their respective tags without the need for additional enzymes. Cell-permeable substrates (SNAP-Cell® and CLIP-Cell™) are suitable for both intracellular and cell-surface labeling, whereas non-cell-permeable substrates (SNAP-Surface® and CLIP-Surface™) are specific for fusion proteins expressed on the cell surface only.

As an alternative, CRISPR editing technologies can be adapted to visualize the synthetic chromosomes and to isolate and purify the synthetic chromosomes prior to delivery to target cells. In this process, unique DNA elements/sequences are incorporated into the synthetic chromosomes during production in the synthetic chromosome production cells. The presence of these unique DNA elements/sequences on the synthetic chromosome permits specific targeting of an engineered, nuclease deficient CRISPR/Cas-fluorescent protein visualization complex (CRISPR/CAS-FP) directly to the synthetic chromosome without binding to native, endogenous chromosomes. Subsequently, the binding of the CRISPR/CAS-FP to the synthetic chromosome provides a means to purify the synthetic chromosome by flow cytometry/flow sorting for eventual delivery into recipient cells. The synthetic chromosome production cells are subjected to mitotic arrest followed by purification of the synthetic chromosome by flow cytometry/flow sorting based on the unique CRISPR-fluorescent tag binding to the synthetic chromosome.

The use of CRISPR/CAS-FP bypasses the need for using potentially mutagenic chromosome dyes and alleviates the potential contamination of dye-stained endogenous chromosomes contaminating preparations of flow-sorted synthetic chromosomes. In addition, purified synthetic chromosomes bound with CRISPR/Cas-FP can be utilized for assessing the efficiency of delivery of flow-sorted synthetic chromosomes into recipient target cells by simple measurement of fluorescent signal quantity in a transfected recipient cell population. The CRISPR/Cas-FP bound synthetic chromosomes also can be utilized to flow sort purify or enrich for synthetic chromosome transfected cells. Fluorescent proteins of particular use include but are not limited to TagBFP, TagCFP, TagGFP2, TagYFP, TagRFP, FusionRed, mKate2, TurboGFP, TurboYFP, TurboRFP, TurboFP602, TurboFP635, or TurboFP650 (all available from Evrogen, Moscow); AmCyan1, AcvGFP1, ZsGreen1, ZsYellow1, mBanana, mOrange, mOrange2, DsRed-Express2, DsRed-Express, tdTomato, DsRed-Monomer, DsRed2, AsRed2, mStrawberry, mCherry, HcRed1, mRaspberry, E2-Crimson, mPlum, Dendra 2, Timer, and PAmCherry (all available from Clontech, Palo Alto, CA); HALO-tags; infrared (far red shifted) tags (available from Promega, Madison, WI); and other fluorescent tags known in the art, as well as fluorescent tags subsequently discovered. For example, in some embodiments, SNAP-tags may be used to identify transfected cells following transfection.

In some embodiments, a safety switch is used to regulate the activity of one or more genes encoded upon and/or expressed from the synthetic chromosome. In some embodiments, the safety switch includes nucleic acid sequences encoding one or more pro apoptotic proteins or regulatory nucleic acids. In some embodiments, one or more genes may be present on the synthetic chromosome, or may be engineered into the target cell intended to carry the synthetic chromosome, to encode counterbalancing anti-apoptotic proteins or regulatory nucleic acids.

Progress in bioengineering of cells for gene-based therapies has been held back by the absence of the one indispensable tool required to address complex polygenicity and/or delivery of large genetic payloads: a stable, non-integrating, self-replicating and biocompatible intracellular platform that ensures controlled expression. The present disclosure provides synthetic chromosomes comprising multiple, regulatable expression cassettes, representing a significant breakthrough in cellular therapeutic technologies and providing the ability to coordinately control and manage expression of large genetic payloads and complex polygenic systems. As described herein, synthetic chromosomes provide a chromosome-vector based bioengineering system that can be readily purified from host (engineering) cells and transferred to recipient (patient) cells by standard transfection protocols. Further provided is the ability to turn off gene expression once therapy is completed and the expression of gene products from the synthetic chromosome is no longer necessary for the patient. An off switch or an inactivation switch may be used if there is an adverse reaction to the expression of the gene products from the synthetic chromosome requiring termination of treatment. For example, a whole-chromosome-inactivation switch may be used, such that expression of genes on the synthetic chromosome are inactivated but the chromosome-containing cells remain alive. Alternatively, a synthetic chromosome bearing therapeutic cell-off switch could be used in a cell-based treatment wherein, if the synthetic chromosome is contained within a specific type of cell and the cells transform into an undesired cell type or migrate to an undesirable location and/or the expression of the factors on the synthetic chromosome is deleterious, the switch can be used to kill the cells containing the synthetic chromosome, specifically.

Chromosome inactivation mechanisms have evolved in nature, to compensate for gene dosage in species in which the sexes have different complements of a sex chromosome. In humans, the homogametic sex is female containing two copies of the X chromosome, whereas the heterogametic sex is male and contains only one copy of an X chromosome in addition to one copy of a Y chromosome. A means to inactivate one X chromosome evolved to ensure that males and females have similar expression of genes from the X chromosome. Inactivation is achieved by expression of a long non-coding RNA called Xist (X-inactive specific transcript) that is essential for initiation of X chromosome inactivation but is dispensable for maintenance of the inactive state of the X chromosome in differentiated cells. Xist acts in cis to induce heterchromatization of the chromosome from which it is expressed. The Xist gene is located within a region on the X chromosome called the X inactivation center (Xic) that spans over 1 megabase of DNA and contains both long non-coding RNAs and protein coding genes necessary and sufficient for initiation of X chromosome inactivation. Xist expression is regulated in part by Tsix, which is transcribed antisense across Xist. Expression of Tsix prevents expression of Xist on the active chromosome and deletion of Tsix leads to skewed X inactivation such that the mutated chromosome is always inactivated. Inactivation occurs whenever there is more than one Xic present in a cell; thus, inactivation of the synthetic chromosome incorporating an Xic or specific Xic gene products would occur regardless of the sex of the cell into which it is introduced. Notably, evidence indicates that Xist-induced silencing also can occur on autosomes. The Xist cDNA has been inducibly expressed on one chromosome 21 in trisomy 21-induced pluripotent stem cells and demonstrated to induce heterochromatization and silencing of that chromosome 21. Because Xic contains all the cis acting elements necessary for Xist expression and subsequent chromosome inactivation, Xic more accurately recapitulates natural silencing. Pluripotency factors expressed in stem cells and induced pluripotent stem cells (iPSCs) prevent Xist expression; therefore, expression of a therapeutic from a synthetic chromosome incorporating Xic would occur in stem cells and be silenced through chromosome inactivation as the cells become differentiated. Thus, embodiments of the invention contemplate inclusion on a synthetic chromosome of an entire Xic region, or inclusion of select regions, including Xist with or without Tsix.

In some embodiments, one or more regulatory switches may be included as 1) whole chromosome inactivating switches (comprising an X chromosome inactivation center (Xic) taken from an X chromosome, and/or specific gene sequences from the Xic, including Xist with or without Tsix) and/or 2) gene expression cassette regulatory switches that do not inactivate the whole synthetic chromosome, but instead regulate expression of one or more individual genes on the synthetic chromosome.

In some embodiments, an independent safety switch based on X-chromosome inactivation is employed, in which expression of an X-inactivation specific transcript (Xist) lncRNA results in inactivation of the hSync chromosome. In some embodiments, the synthetic chromosome comprises an entire Xic region from an X chromosome, and in other embodiments, the synthetic chromosome comprises select sequences from the Xic region of the X chromosome, including the Xist locus, and in some embodiments, further comprising a Tsix locus.

In some embodiments, a regulatory RNA (e.g., an inhibitory RNA) may be produced by induction of the promoter. In some embodiments, a regulatory RNA may be used to regulate an endogenous gene product, or a promoter or a transcript produced by the synthetic chromosome.

As used herein, the term “Xic” refers to sequences at the X inactivation center present on the X chromosome that control the silencing of that X chromosome. As used herein, the term “Xist” refers to the X-inactive specific transcript gene that encodes a large non-coding RNA that is responsible for mediating silencing of the X chromosome from which it is transcribed. “Xist” refers to the RNA transcript. As used herein, the term “Tsix” refers to a gene that encodes a large RNA which is not believed to encode a protein. “Tsix” refers to the Tsix RNA, which is transcribed antisense to Xist; that is, the Tsix gene overlaps the Xist gene and is transcribed on the opposite strand of DNA from the Xist gene. Tsix is a negative regulator of Xist. As used herein, the term “Xic” also refers to genes and nucleic acid sequences derived from nonhuman species and human gene variants with homology to the sequences at the X inactivation center present on the X chromosome that control the silencing of that X chromosome in humans.

In some embodiments, the Xic or select Xic gene product expression cassette is inserted into a synthetic chromosome to provide transcriptional and translational regulatory sequences, and in some embodiments provides for inducible or repressible expression of Xic gene products. In general, the transcriptional and translational regulatory sequences may include, but are not limited to, promoter sequences, ribosomal binding sites, transcriptional start and stop sequences, translational start and stop sequences, repressible sequences, and enhancer or activator sequences.

In general, the regulatable (inducible/repressible) promoters of use in the present invention are not limited, as long as the promoter is capable of inducing (i.e., “turning on” or “upregulating”) or repressing (i.e., “turning off” or “downregulating”) expression of the downstream gene in response to an external stimulus. One such system involves tetracycline controlled transcriptional activation where transcription is reversibly turned on (Tet-On) or off (Tet-Off) in the presence of the antibiotic tetracycline or a derivative thereof, such as doxycycline. In a Tet-Off system, expression of tetracycline response element-controlled genes can be repressed by tetracycline and its derivatives. Tetracycline binds the tetracycline transactivator protein, rendering it incapable of binding to the tetracycline response element sequences, preventing transactivation of tetracycline response element-controlled genes. In a Tet-On system on the other hand, the tetracycline transactivator protein is capable of initiating expression only if bound by tetracycline; thus, introduction of tetracycline or doxycycline initiates the transcription of the Xic gene product in toto or specific Xic genes. Another inducible promoter system known in the art is the estrogen receptor conditional gene expression system. Compared to the Tet system, the estrogen receptor system is not as tightly controlled; however, because the Tet system depends on transcription and subsequent translation of a target gene, the Tet system is not as fast-acting as the estrogen receptor system. Alternatively, a Cumate Switch Inducible expression system—in the repressor configuration—may be employed. The Cumate Switch Inducible expression system is based on the bacterial repressor controlling the degradative pathway for p-cymene in Pseudomonas putida. High levels of the reaction product, p-cumate, allow binding of the repressor CymR to the operator sequences (CmO) of the p-cym and p-cmt operon. Other regulatable (inducible/repressible) systems employing small molecules are also envisioned as useful in the methods and compositions of the present disclosure.

The entire Xic region may be loaded on to the synthetic chromosome due to the ability of synthetic chromosomes to accommodate very large genetic payloads (>100 Kilo basepairs and up to Megabasepairs (Mbps) in length), or select regions from Xic may be used, including Xist with or without Tsix. The Tsix-Xist genomic region is located on the long arm of the X chromosome at Xq13.2. The Xist and Tsix long non-coding RNAs are transcribed in antisense directions. The Xist gene is over 32 Kb in length while the Tsix gene is over 37 Kb in length. In addition, the entire X chromosome inactivation center, Xic (>1 Mbp in size), may be loaded onto the synthetic chromosome, e.g., as a series of overlapping, engineered BACs.

Illustrative publications describing components of precursor compositions, as well as methods for preparing certain compositions include the following:

Incorporated by reference in their entirety are: U.S. Patent Publication Nos. US2018/0010150 (Ser. No. 15/548,236); US2020/0157553 (Ser. No. 16/092,828); US2019/0345259 (U.S. Ser. No. 16/092,841); US2020/0131530 (U.S. Ser. No. 16/494,252); US2018/0171355 (U.S. Ser. No. 15/844,014); US2019/0071738 (U.S. Ser. No. 16/120,638); and PCT Publication WO 2017/180665 (U.S. Ser. No. 16/092,837).

Certain patents and patent application publications of interest to the present disclosure and incorporated by reference in their entirety are: U.S. Pat. No. 8,709,404 (describing method of cancer immunotherapy in which lymphocytes are collected from sentinel lymph nodes and cultured and expanded in vitro); U.S. Pat. No. 8,101,173 (describing an immunotherapeutic method for treating a patient suffering from urinary bladder cancer by administering expanded tumor-reactive T-lymphocytes from sentinel lymph nodes draining a tumor in the bladder, and/or metinel lymph nodes (metastasis-draining lymph nodes draining a metastasis arising from a tumor in the bladder); and U.S. Pat. No. 8,206,702 (describing a method useful in treating and/or preventing cancer in which tumor-reactive lymphocytes, such as CD4⁺ helper and/or CD8⁺ T-lymphocytes, are stimulated with tumor-derived antigen and at least one substance having agonistic activity towards the IL-2 receptor to promote survival, growth/expansion, a second phase is initiated when the CD25 cell surface marker (or IL-2R marker) is down-regulated on CD4⁺ T helper and/or CD8⁺ T-lymphocytes).

As used herein, a “sentinel node” is defined as the first tumor-draining lymph node along the direct drainage route from the tumor, and in case of dissemination it is considered to be the first site of metastasis. As used herein, “metinel nodes” are metastasis-draining lymph nodes draining a metastasis.

Also of note are recent advances in surgery and basic immunology and the identification of a natural immune response harbored in sentinel nodes, tumor draining lymph nodes. The sentinel node is rich in tumor-recognizing T lymphocytes for expansion and use in immunotherapy. Lymphocytes acquired from the sentinel node can be used in adoptive immunotherapy of colon cancer.

Researchers conducted a flow cytometric investigation of tumor draining lymph node (sentinel node) derived B cell activation by autologous tumor extract in patients with muscle invasive urothelial bladder cancer (MIBC), and results indicated the potential for enhanced survival of patients with MIBC, which had remained around 50% (5 years) using combined radical surgery and neoadjuvant chemotherapy. Sentinel nodes (SNs) from 28 patients with MIBC were detected by a Geiger meter at cystectomy after peritumoral injection with radioactive isotope. Lymphocytes were isolated from freshly received SNs where they were stimulated with autologous tumor extract in a sterile environment. After cultivation for 7 days, the cells were analyzed by multi-color flow cytometry using FASCIA (Flow cytometric Assay of Specific Cell-mediated Immune response in Activated whole blood). Patients displayed an increased B cell activation in SNs after stimulation with autologous tumor extract compared to when SN acquired lymphocytes were stimulated with autologous extract of macroscopically non-malignant bladder. CD4⁺ T cells from SNs were activated and formed blasts after co-culture with SN acquired B cells in the presence of tumor antigen. However, CD4⁺ T cells were not activated and did not blast when co-cultured with B cells incubated with HLA-DR-blocking antibodies, indicating the antigen presenting ability of SN acquired B cells. SN-acquired B lymphocytes can be activated in culture upon stimulation with autologous tumor extract but not with extract of non-malignant epithelium of the bladder, after 7 days. A lower number of SN-acquired CD4⁺ T cells cultured with HLA-DR blocked CD19⁺ cells in presence of tumor antigen, indicating functional antigen presenting ability of B cells in sentinel nodes. Thus, in vitro expansions of sentinel node-acquired autologous tumor specific CD4⁺ T cells showed promise for adoptive immunotherapy. Researchers also reported that naive T helper cells need effective APCs presenting tumor antigens to become activated. These researchers observed that B cells in cancer patients were tumor-antigen experienced, and from their phenotypes a CD4⁺ T cell dependent anti-tumoral response was suggested.

Also of interest is a report showing that infusion of expanded, autologous, tumor specific T-helper cells is a potential treatment option in metastasized urinary bladder cancer.

Also of interest as useful components of the synthetic chromosome are sequences encoding Chimeric antigen receptor T cells (also known as CARs, CAR T cells, chimeric immunoreceptors, chimeric T cell receptors or artificial T cell receptors). CAR T cells have been genetically engineered to combine both antigen-binding and T cell activating functions into a single receptor, thereby producing an artificial T cell receptor that can be used in immunotherapy, because they are receptor proteins engineered to target T cells to a specific protein ligand. In some embodiments, cells carrying synthetic chromosomes may encode one or multiple modified chimeric antigen receptor (CAR) genes, and these synthetic chromosome carrying cells may be used as cellular therapeutic agents.

CARs are composed of an extracellular binding domain, a hinge region, a transmembrane domain, and at least one intracellular signaling domain (CD3ζ chain domain). Single-chain variable fragments (scFvs) derived from tumor antigen-reactive antibodies are commonly used as extracellular binding domains in CARs. Second- or third-generation CARs also contain costimulatory domains, like CD28 and/or 4-1BB, to improve proliferation, cytokine secretion, resistance to apoptosis, and in vivo persistence. Third-generation CARs exhibit improved effector functions and in vivo persistence as compared to second-generation CARs, whereas fourth-generation CARs, so-called TRUCKs or armored CARs, combine the expression of a second-generation CAR with factors that enhance anti-tumoral activity, such as cytokines, costimulatory ligands, or enzymes that degrade the extracellular matrix of solid tumors. So-called smart T cells may also be equipped with a “suicide gene” or include synthetic control devices to enhance the safety of CAR T cell therapy. (Hartmann et al., 2017, EMBO Mol. Med., 9(9):1183-1197).

### Cell Types

Synthetic chromosomes of the present disclosure are created in cultured cells in vitro before the synthetic chromosome is then used to transfect target cells. Potential cells of use include any living cell, but those from eukaryotes, most often mammalian cells, are specifically contemplated. Cells from humans are specifically contemplated. In some embodiments, the cells used to engineer and produce the synthetic chromosome can be cells naturally occurring in a subject (human patient, animal or plant). In some embodiments, the cell line comprises endogenous, heterologous and/or bioengineered genes or regulatory sequences that interact with and/or bind to nucleic acid sequences integrated into the synthetic chromosome.

The target cells can also be engineered to incorporate one or more safety switches, which can inactivate specific genes on or the entire synthetic chromosome or can initiate an apoptotic pathway to specifically kill cells comprising the synthetic chromosome. One such safety switch may employ an X inactivation center (Xic), or one or more genes from Xic. The Xic or Xic genes may be engineered into the cell line, and/or into the synthetic chromosome by any method currently employed in the art.

Gene expression regulatory systems and/or synthetic chromosome-bearing therapeutic cell-off safety switches can be designed to employ genes involved in apoptosis as components on the synthetic chromosome for use of the cell+bioengineered chromosome compositions in treating immune responses to infection, autoimmune diseases, and cancer. Apoptotic signalling pathways include (i) an extrinsic pathway, in which apoptosis is initiated at the cell surface by ligation of death receptors resulting in the activation of caspase-8 at the death inducing signalling complex (DISC) and, in some circumstances, cleavage of the BH3—only protein BID; and (ii) an intrinsic pathway, in which apoptosis is initiated at the mitochondria and is regulated by BCL2-proteins. Activation of the intrinsic pathway results in loss of mitochondrial membrane potential, release of cytochrome c, and activation of caspase-9 in the Apaf-1 containing apoptosome. Both pathways converge into the activation of the executioner caspases, (e.g., caspase 3). Caspases may be inhibited by the Inhibitor of apoptosis proteins (IAPs). The activities of various antiapoptotic BCL-2 proteins and their role in solid tumors is under active research, and several strategies have been developed to inhibit BCL2, BCL-XL, BCLw, and MCL1. Studies of several small molecule BCL-2 inhibitors (e.g., ABT-737, ABT-263, ABT-199, TW-37, sabutoclax, obatoclax, and MIM1) have demonstrated their potential to act as anticancer therapeutics. The BCL2-family includes: the multidomain pro-apoptotic proteins BAX and BAK mediating release of cytochrome c from mitochondria into cytosol. BAX and BAK are inhibited by the antiapoptotic BCL2-proteins (BCL2, BCL-XL, BCL-w, MCL1, and BCL2A1). BH3-only proteins (e.g., BIM, BID, PUMA, BAD, BMF, and NOXA) can neutralize the function of the antiapoptotic BCL2-proteins and may also directly activate BAX and BAK.

Bcl-2 proteins can be further characterized as having antiapoptotic or pro-apoptotic function, and the pro-apoptotic group is further divided into BH3-only proteins (‘activators’ and ‘sensitizers’) as well as non-BH3-only ‘executioners’. Enhanced expression and/or post transcriptional modification empowers ‘activators’ (Bim, Puma, tBid and Bad) to induce a conformational change in ‘executioners’ (Bax and Bak) to polymerize on the surface of mitochondria, thereby creating holes in the outer membrane and allowing cytochrome c (cyto c) to escape from the intermembrane space. In the cytoplasm, cyto c initiates the formation of high-molecular-weight scaffolds to activate dormant caspases, which catalyze proteolytic intracellular disintegration. Destruction of the cell culminates in the formation of apoptotic bodies that are engulfed by macrophages. Antiapoptotic Bcl-2 proteins like Bcl-2, Mcl-1, Bcl-XL and A1, also known as ‘guardians’, interfere with the induction of apoptosis by binding and thereby neutralizing the pro-apoptotic members.

Target cells can be primary-culture cell lines established for the purpose of synthetic chromosome production specific for an individual. Alternatively, in some embodiments, the cells to be engineered and/or produce the synthetic chromosome are from an established cell line.

Also contemplated are embryonic cell lines; pluripotent cell lines; adult derived stem cells; or broadly embryonic or reprogrammed cell lines. Further contemplated are primary or cultured cell lines from domesticated pet, livestock and/or agriculturally significant animals, such as dogs, cats, rabbits, hares, pikas, cows, sheep, goats, horses, donkeys, mules, pigs, chickens, ducks, fishes, lobsters, shrimp, crayfish, eels, or any other food source animal or plant cell line of any species. Specifically contemplated are avian, bovine, canine, feline, porcine and rodent (rats, mice, etc.) cells, as well as cells from any ungulate, e.g., sheep, deer, camel goat, llama, alpaca, zebra, or donkey. Cell lines from eukaryotic laboratory research model systems, such as Drosophila and zebrafish, are specifically contemplated. Primary cell lines from zebras, camels, dogs, cats, horses, and chickens (e.g., chicken DT40 cells), are specifically contemplated.

Also contemplated are methods of rescuing wildlife or endangered species (polar bears, ringed seals, spider monkeys, tigers, whales, sea otters, sea turtles, bison, for example) at risk of becoming extinct due to factors such as habitat loss (e.g., due to invasion of another species, human development and/or global warming) or poaching. Species (plant or animal) that may become endangered and may be in need of rescue due to global warming trends are explicitly contemplated. Also contemplated is the use of the presently claimed cell+synthetic chromosome composition to engineer plant cells to become more nutritive, such as engineering crop plant cells to comprise synthetic chromosomes to carry one or more genes (i) enhancing survival of the plant cell, and/or (ii) enhancing its nutritive value when the plant is eaten.

In some embodiments, the preferred cell lines are mammalian. In some embodiments, the cell lines are human. In some embodiments, the cell lines are from domesticated animals or agricultural livestock. In some embodiments, the cell lines are mesenchymal stem cells, including human mesenchymal stem cells (hMSCs). In some embodiments, the cell lines are pluripotent or induced pluripotent stem cells (iPSCs).

In some embodiments, the cells to be engineered and/or produce the synthetic chromosome are from an established cell line. A wide variety of cell lines for tissue culture are known in the art. Examples of cell lines include but are not limited to human cells lines such as 293-T (embryonic kidney), 721 (melanoma), A2780 (ovary), A172 (glioblastoma), A253 (carcinoma), A431 (epithelium), A549 (carcinoma), BCP-1 (lymphoma), BEAS-2B (lung), BR 293 (breast), BxPC3 (pancreatic carcinoma), Cal-27 (tongue), COR-L23 (lung), COV-434 (ovary), CML T1 (leukemia), DUl45 (prostate), DuCaP (prostate), eHAP fully haploid engineered HEK293/HeLa wild-type cells, FM3 (lymph node), H1299 (lung), H69 (lung), HCA2 (fibroblast), HEK0293 (embryonic kidney), HeLa (cervix), HL-60 (myeloblast), HMEC (epithelium), HT-29 (colon), HT1080 (fibrosarcoma), HUVEC (umbilical vein epithelium), Jurkat (T cell leukemia), JY (lymphoblastoid), K562 (lymphoblastoid), KBM-7 (lymphoblastoid), Ku812 (lymphoblastoid), KCL22 (lymphoblastoid), KGI (lymphoblastoid), KYO1 (lymphoblastoid), LNCap (prostate), Ma-Mel (melanoma), MCF-7 (mammary gland), MDF-10A (mammary gland), MDA-MB-231, -468 and -435 (breast), MG63 (osteosarcoma), MOR/0.2R (lung), MONO-MAC6 (white blood cells), MRC5 (lung), NCI-H69 (lung), NALM-1 (peripheral blood), NW-145 (melanoma), OPCN/OPCT (prostate), Peer (leukemia), Raji (B lymphoma), Saos-2 (osteosarcoma), Sf21 (ovary), Sf9 (ovary), SiHa (cervical cancer), SKBR3 (breast carcinoma), SKOV-2 (ovary carcinoma), T-47D (mammary gland), T84 (lung), U373 (glioblastoma), U87 (glioblastoma), U937 (lymphoma), VCaP (prostate), WM39 (skin), WT-49 (lymphoblastoid), and YAR (B cell). In some embodiments non-human cell lines may be employed. Rodent cell lines of interest include but are not limited to 3T3 (mouse fibroblast), 4T1 (mouse mammary), 9 L (rat glioblastoma), A20 (mouse lymphoma), ALC (mouse bone marrow), B16 (mouse melanoma), B35 (rat neuroblastoma), bEnd.3 (mouse brain), C2C12 (mouse myoblast), C6 (rat glioma), CGR8 (mouse embryonic), CT26 (mouse carcinoma), E14Tg2a (mouse embryo), EL4 mouse leukemia), EMT6/AR1 (mouse mammary), Hepa1c1c7 (mouse hepatoma), J558L (mouse myeloma), MC-38 (mouse adenocarcinoma), MTD-1A (mouse epithelium), RBL (rat leukemia), RenCa (mouse carcinoma), X63 (mouse lymphoma), YAC-1 (mouse Be cell), BHK-1 (hamster kidney), DG44 Chinese Hamster Ovary cell line, and CHO (hamster ovary). Plant cell lines of use include but are not limited to BY-2, Xan-1, GV7, GF11, GT16, TBY-AtRER1B, 3n-3, and G89 (tobacco); VR, VW, and YU-1 (grape); PAR, PAP, and PAW (pokeweed); Spi-WT, Spi-1-1, and Spi12F (spinach); PSB, PSW and PSG (sesame); A.per, A.pas, A.plo (asparagus); Pn and Pb (bamboo); and DG330 (soybean). These cell lines and others are available from a variety of sources known to those with skill in the art (see, e.g., the American Type Culture Collection (ATCC) (Manassas, Va.)). These cell lines and others are available from a variety of sources known to those with skill in the art (see, e.g., the American Type Culture Collection (ATCC) (Manassas, Va.)).

Of particular interest are patient autologous cell lines, allogeneic cells, as well as cell lines from a heterologous patient with a similar condition to be treated. In some embodiments, the HT1080 human cell line is employed.

A cell transfected with one or more vectors described herein is used to establish a new cell line, which may comprise one or more vector-derived sequences. The synthetic chromosome producing cell line can then be maintained in culture, or alternatively, the synthetic chromosome(s) can be isolated from the synthetic chromosome producing cell line and transfected into a different cell line for maintenance before ultimately being transfected into a target cell, such as a mammalian cell.

### Synthetic Chromosome Production

The synthetic chromosomes of the present disclosure may be produced by any currently employed methods of synthetic chromosome production. As discussed briefly, above, the real-time monitoring methods of the present invention are applicable to all of the “bottom up”, “top down”, engineering of minichromosomes, and induced de novo chromosome generation methods used in the art.

The “bottom up” approach of synthetic chromosome formation relies on cell-mediated de novo chromosome formation following transfection of a permissive cell line with cloned a satellite sequences, which comprise typical host cell-appropriate centromeres and selectable marker gene(s), with or without telomeric and genomic DNA. Both synthetic and naturally occurring a-satellite arrays, cloned into yeast artificial chromosomes, bacterial artificial chromosomes, or P1-derived artificial chromosome vectors have been used in the art for de novo synthetic chromosome formation. The products of bottom-up assembly can be linear or circular, comprise simplified and/or concatamerized input DNA with an a-satellite DNA based centromere, and typically range between 1 and 10 Mb in size. Bottom up-derived synthetic chromosomes also are engineered to incorporate nucleic acid sequences that permit site specific integration of target DNA sequences onto the synthetic chromosome.

The “top down” approach of producing synthetic chromosomes involves sequential rounds of random and/or targeted truncation of pre-existing chromosome arms to result in a pared down synthetic chromosome comprising a centromere, telomeres, and DNA replication origins. “Top down” synthetic chromosomes are constructed optimally to be devoid of naturally occurring expressed genes and are engineered to contain DNA sequences that permit site specific integration of target DNA sequences onto the truncated chromosome, mediated, e.g., by site-specific DNA integrases.

A third method of producing synthetic chromosomes known in the art is engineering of naturally occurring minichromosomes. This production method typically involves irradiation induced fragmentation of a chromosome containing a neocentromere possessing centromere activity in human cells yet lacking a-satellite DNA sequences and engineered to be devoid of non-essential DNA. As with other methods for generating synthetic chromosomes, minichromosomes can be engineered to contain DNA sequences that permit site-specific integration of target DNA sequences.

The fourth approach for production of synthetic chromosomes involves induced de novo chromosome generation by targeted amplification of specific chromosomal segments. This approach involves large-scale amplification of pericentromeric/ribosomal DNA regions situated on acrocentric chromosomes. The amplification is triggered by co-transfection of excess exogenous DNA specific to the pericentric region of chromosomes, e.g., ribosomal RNA, along with DNA sequences that allow for site-specific integration of target DNA sequences and also a selectable marker, which integrates into the pericentric heterochromatic regions of acrocentric chromosomes. During this process, upon targeting and integration into the pericentric regions of the acrocentric chromosomes, the co-transfected DNA induces large-scale amplification of the short arms of the acrocentric chromosome (rDNA/centromere region), resulting in duplication/activation of centromere sequences, formation of a dicentric chromosome with two active centromeres, and subsequent mitotic events result in cleavage and resolution of the dicentric chromosome, leading to a “break-off” satellite DNA-based synthetic chromosome approximately 40-80 Mb in size comprised largely of satellite repeat sequences with subdomains of co-amplified transfected transgene that may also contain amplified copies of rDNA, as well as multiple site-specific integration sites. The newly-generated synthetic chromosome can be validated by observation of fluorescent chromosome painting or FISH or FlowFISH or CASFISH (via markers that have been incorporated, such as an endogenous chromosome tag and a synthetic chromosome tag, which were engineered into the synthetic chromosome production cell line and/or the synthetic chromosome itself, as the synthetic chromosome was being made.

An artificial chromosome expression system (ACE system) has been described previously as a means to introduce large payloads of genetic information into the cell. Synthetic or ACE platform chromosomes are synthetic chromosomes that can be employed in a variety of cell-based protein production, modulation of gene expression or therapeutic applications. During the generation of synthetic platform chromosomes, unique DNA elements/sequences required for integrase mediated site-specific integration of heterologous nucleic acids are incorporated into the synthetic chromosome which allows for engineering of the synthetic chromosome. By design, and because the integrase targeting sequences are amplified during synthetic chromosome production, a large number of site-specific recombination sites are incorporated onto the synthetic chromosome and are available for the multiple loading of the synthetic platform chromosome by delivery vectors containing multiple gene regulatory control systems.

Thus, the ACE System consists of a platform chromosome (ACE chromosome) containing approximately 75 site-specific recombination acceptor sites that can carry single or multiple copies of genes of interest using specially designed ACE targeting vectors (pAPP) and a site-specific integrase (ACE Integrase). The ACE Integrase is a derivative of the bacteriophage lambda integrase (INT) engineered to direct site-specific unidirectional recombination in mammalian cells in lieu of bacterial encoded, host integration accessory factors (λINTR). Use of a unidirectional integrase allows for multiple and/or repeated integration events using the same, recombination system without risking reversal (i.e., pop-out) of previous integration/insertions of bioengineered expression cassettes. The transfer of an ACE chromosome carrying multiple copies of a red fluorescent protein reporter gene into human MSCs has been demonstrated. Fluorescent in situ hybridization and fluorescent microscopy demonstrated that the ACEs were stably maintained as single chromosomes and expression of transgenes in both MSCs and differentiated cell types is maintained.

### Chromosome Transfer

Adipose-derived MSCs can be obtained from Lonza and cultured as recommended by the manufacturer, in which the cells are cultured under a physiological oxygen environment (e.g., 3% O2). A low oxygen culture condition more closely recapitulates the in vivo environment and has been demonstrated to extend the lifespan and functionality of MSCs. Engineered platform chromosomes can be purified away from the endogenous chromosomes of the synthetic chromosome production cells by high-speed, flow cytometry and chromosome sorting, for example, and then delivered into MSCs by commercially available lipid-based transfection reagents. Delivery of intact, engineered ACE platform chromosomes can be confirmed by FISH, Flow-FISH, CASFISH and/or PCR analysis.

Functional Elements which May be Integrated into the Synthetic Chromosome:

### 1. Coordinated Expression of Multiple Genes in a Biochemical Pathway as “Cellular Enhancements” for Cellular Gene Therapy

The use of a synthetic chromosome able to carry extremely large inserts allows for the expression of multiple expression cassettes comprising large genomic sequences, and multiple genes comprising entire biosynthetic pathways, for example. As one example, several genes involved in a biosynthetic pathway can be inserted onto and expressed from the synthetic chromosome to confer upon the cells in which the synthetic chromosome resides an ability to produce cellular metabolites such as amino acids, nucleic acids, glycoproteins and the like. Thus, a synthetic chromosome-carrying cell's ability to produce such metabolites can be orchestrated by the coordinated expression of multiple gene products that make up the biochemical pathway for metabolite synthesis. In some disease states, mammalian cells lack one or more enzymes needed to make essential amino acids; to enable cells to make these amino acids, cells can be engineered to express heterologous genes found in fungi or bacteria. Previously, multiple iterations of transfection or transduction events were necessary in order to generate an entire biochemical or biosynthetic pathway in the recipient cells. Furthermore, viral-based systems, plasmid-based systems, bacterial artificial chromosomes (BACs), and even some previously dubbed “mammalian artificial chromosomes (MACs)” or “human artificial chromosomes (HACs)” were inadequate as delivery systems for various reasons, such as their limited payload capacity, instability over generations of cell division, propensity to rearrangements, lack of engineerability and/or portability of the alleged “chromosome” into target cells. The hSyncs described herein are easily bioengineered and are readily portable from one cell or cell type into other cells.

As one non-limiting example of a disease that could be treated using the therapeutic composition disclosed herein, Niemann-Pick is a rare, inherited disease that affects the body's ability to metabolize fat (cholesterol and lipids) within cells. Niemann-Pick disease is divided into four main types: type A, type B, type C1, and type C2. Overall, Niemann-Pick diseased cells malfunction and die over time. Types A and B of Niemann-Pick disease are caused by mutations in the SMPD1 gene, which encodes an enzyme called acid sphingomyelinase found in lysosomes, the waste disposal and recycling compartments within cells. Affected children can be identified in an eye examination, as they have an eye abnormality called a cherry-red spot. Infants with Niemann-Pick disease type A usually develop an enlarged liver and spleen (hepatosplenomegaly) by age 3 months and fail to gain weight and grow at the expected rate (failure to thrive). Affected children with type A develop normally until around age 1 year when they experience a progressive loss of mental abilities and movement (psychomotor regression); these children also develop widespread lung damage (interstitial lung disease) that can cause recurrent lung infections and eventually lead to respiratory failure. Children with Niemann-Pick disease type A generally do not survive past early childhood.

Niemann-Pick disease type B usually presents in mid-childhood. About one-third of affected individuals have the cherry-red spot eye abnormality or neurological impairment. The signs and symptoms of this type are similar to, but less severe than, type A. People with Niemann-Pick disease type B often have hepatosplenomegaly, recurrent lung infections, and a low number of platelets in the blood (thrombocytopenia). They also have short stature and slowed mineralization of bone (delayed bone age). People with Niemann-Pick disease type B usually survive into adulthood.

Niemann-Pick type C (NPC) disease is a panethnic lysosomal lipidosis resulting in severe cerebellar impairment and death and is proposed to be a consequence of defective metabolite transport. The signs and symptoms of Niemann-Pick disease types C1 and C2 are very similar; these types differ only in their genetic cause. Niemann-Pick disease types C1 and C2 usually become apparent in childhood, although signs and symptoms can develop at any time. People with these types usually develop difficulty coordinating movements (ataxia), an inability to move the eyes vertically (vertical supranuclear gaze palsy), poor muscle tone (dystonia), severe liver disease, and interstitial lung disease. Individuals with Niemann-Pick disease types C1 and C2 have problems with speech and swallowing that worsen over time, eventually interfering with feeding. Affected individuals often experience progressive decline in intellectual function and about one-third have seizures. People with these types may survive into adulthood.

Niemann-Pick disease is an example of a disease that can be treated by supplying multiple genes in the biochemical pathway (e.g., sphingomyelinase, as well as other metabolites and/or components of the lysosomal pathway that are defective and lead to Niemann-Pick lipidosis) to correct the pathway. The bioengineered hSync is used to transfect mesenchymal (or other) stem cells, and the therapeutic cell composition is administered to the individuals affected by Niemann-Pick to provide cells that properly metabolize lipids and cholesterol due to the expression of the necessary genes from the bioengineered hSync, thereby correcting the lysosomal transport and/or processing defects using the therapeutic cell composition.

Another example of a cellular environment enhancement provided by the cell+bioengineered synthetic chromosome compositions disclosed herein, the synthetic chromosomes may be engineered to comprise multiple genes capable of effectuating tryptophan biosynthesis, such as the five genes necessary for synthesis of tryptophan in Saccharomyces cerevisiae. Indoleamine 2,3-dioxygenase (IDO) is the first and rate-limiting enzyme of tryptophan catabolism through the kynurenine pathway. The IDO enzyme is believed to play a role in mechanisms of tolerance; one of its physiological functions the suppression of potentially dangerous inflammatory processes in the body, as well as in cancer. IDO is expressed in tumors and tumor-draining lymph nodes and degrades tryptophan (Trp) to create an immunosuppressive micro milieu both by depleting Trp from the tumor environment, and by accumulating immunosuppressive metabolites of the kynurenine (kyn) pathway, preventing non-cancerous cells in the same milieu from surviving. Clinical studies have tested 1-methyl-D-tryptophan (1-D-MT) in patients with relapsed or refractory solid tumors with the aim of inhibiting IDO-mediated tumor immune escape. According to one study, proliferation of alloreactive T-cells co-cultured with IDO1-positive human cancer cells was actually inhibited by 1-D-MT; furthermore, incubation with 1-D-MT increased kyn production. It was found that 1-D-MT did not alter IDO1 enzymatic activity, but rather, 1-D MT induced IDO1 mRNA and protein expression through pathways involving p38 MAPK and JNK signalling. Thus, treatment of cancer patients with 1-D-MT has transcriptional effects that may promote rather than suppress anti-tumor immune escape by increasing IDO1 in the cancer cells. Such off-target effects should be carefully analyzed in the ongoing clinical trials with 1-D-MT. In some embodiments, the cell+bioengineered synthetic chromosome composition is used to prevent T cell exhaustion by providing on the synthetic chromosome all of the genes necessary for the tryptophan biosynthetic pathway.

In some aspects, in addition to delivering the multiple genes capable of effectuating a biosynthetic pathway, the delivery vector further comprises one or more of a) one or more genes that interfere with or block tumor cell ability to inhibit immune cell cycle progression, b) one or more genes that code for factors that enhance immune cell activation and growth, or c) one or more genes that increase specificity of immune cells to developing tumors.

In some aspects, the method further comprises the steps of: isolating the synthetic chromosome expressing the biosynthetic pathway; and transferring the synthetic chromosome to a second recipient cell. In some aspects, the second recipient cell is selected from a universal donor T-cell or a patient autologous T-cell. Other aspects of the invention provide the synthetic chromosome expressing the biosynthetic pathway, and yet other aspects provide the second recipient cell.

### 2. Complex, Coordinately Regulated Biological Circuits

Another use of the synthetic chromosome is to encode the multiple components of a complex and interdependent biological circuit, expression of which components can be coordinately regulated for specific expression, spatially (targeted to specific tissues or tumor environments), temporally (such as induction or repression of expression, in a particular sequence), or both. Thus, the present invention encompasses compositions and methods to allow one to deliver and express multiple genes from multiple gene regulatory control systems all from a single synthetic chromosome.

For example, in some embodiments, the compositions and methods of the present disclosure comprise a synthetic chromosome expressing a first target nucleic acid under control of a first regulatory control system; and a second target nucleic acid under control of a second regulatory control system. In some embodiments, the synthetic chromosome expresses the first target nucleic acid under control of the first regulatory control system and the second target nucleic acid under control of the second regulatory control system.

In some embodiments, the method can comprise a step of inducing transcription of the first and second target nucleic acids via the first and second regulatory control systems.

In some embodiments a gene product of the first target nucleic acid regulates transcription of a second target nucleic acid. In some embodiments, the gene product of the first target nucleic acid induces transcription of the second target nucleic acid; and in some embodiments, the gene product of the first target nucleic acid suppresses transcription of the second target nucleic acid.

Thus, in some embodiments, the method can comprise inducing transcription of the first target nucleic acid via the first regulatory control system to produce the first gene product and regulating transcription of the second target nucleic acid via the first gene product.

The cells containing the synthetic chromosome may comprise first, second and third target nucleic acids, wherein each of the first, second and third target nucleic acids is under control of an independent regulatory control system.

Still other embodiments of the present compositions and methods may involve engineering a recipient cell with at least three target nucleic acids, each under control of a regulatory control system that is complex and interdependent. For example, the gene products of the first and second target nucleic acids can act together to regulate transcription of the third target nucleic acid via the third regulatory control system. Accordingly, in some embodiments, transcription of the first and second target nucleic acids via the first and second regulatory control systems is induced produce the first and second gene products, wherein the first and second gene products act together to regulate (induce or repress) transcription of the third target nucleic acid. In one aspect of this embodiment, both the first and second gene products are necessary to regulate transcription of the third target nucleic acid; in another embodiment, either the first or the second gene product regulates transcription of the third target nucleic acid. In some embodiments, regulation of the third target nucleic acid is inducing transcription of the third target nucleic acid, and in other embodiments, regulation of the third target nucleic acid is suppressing transcription of the third target nucleic acid.

In certain aspects of all the embodiments, the first, second and/or third regulatory control systems may be selected from the group consisting of a Tet-On, Tet-Off, Lac switch inducible, ecdysone-inducible, cumate gene-switch and a tamoxifen-inducible system.

Additionally, aspects of all embodiments include the isolated cells comprising the synthetic chromosomes comprising the first; the first and second; and/or the first, second and third target nucleic acids; as well as the synthetic chromosomes upon which are loaded the first; the first and second; and the first, second and third target nucleic acids.

For example, a biological circuit may be included on a synthetic chromosome to provide amplification of signal output. In some embodiments, there is no production of either gene product 1 or gene product 2 when inducer 1 is absent. However, when inducer 1 is present, gene 1 is transcribed, gene product 1 is expressed, and gene product 1 in turn induces the transcription and translation of gene 2 and the synthesis of gene product 2. One example of a use of this embodiment is the concerted expression of multiply-loaded genes that confer increased and enhanced cell and/or whole animal survival. In this scenario, multiply-loaded genes are positioned and expressed from a synthetic chromosome that confers increased immune cell survival in response to tumor challenge. Tumor cells employ a variety of means to escape recognition and reduce T-cell function; however, this challenge may be circumvented by engineering T-cells to express from a common regulatory control system multiply-loaded factors that inhibit cell cycle arrest response; e.g., expression of genes that code for inhibitors to the immune and cell cycle checkpoint proteins, such as anti-PD-1 (programmed cell death protein 1) and anti-CTLA-4 (central T-Cell activation and inhibition 4). Thus, from one inducing regulatory control system, multiple gene products can be produced to enhance immune cell function.

In other embodiments of the present invention, more complex “logic” circuits are constructed. For example, a logical “AND” switch can be built such that the expression of two genes and the production of two gene products leads to the expression of a third gene and a production of a third product.

In another embodiment, a logical “OR” switch is constructed whereby the presence of inducer 1 OR inducer 2 can lead to the expression of gene 1 or gene 2, the production of gene product 1 or gene product 2, and the expression of gene 3 and production of gene product 3. Such circuits and logical switches (“AND”/“OR”) outlined above also may be coordinated to function with endogenous cellular inducers or inducers encoded on additional exogenous DNA (e.g., vectors aside from the synthetic chromosome) residing in the cell. For example, a regulatory control system could be engineered on the synthetic chromosome to respond to exogenous signals emanating from the tissue environment, such as an IL-2 responsive promoter driving expression of a factor (e.g. an anti-tumor factor) that would be expressed in a tumor microenvironment.

In some embodiments the therapeutic agent, therapeutic composition, or the synthetic chromosome is under expression control of an endogenous regulatory factor. In one such aspect, the therapeutic agent, therapeutic composition, and/or the synthetic chromosome could be engineered to respond to a signal produced by cancerous cells; thus, the therapeutic agent, therapeutic composition, and/or the synthetic chromosome can be engineered to be self-titrating, minimizing any potential risks of toxicity to the subject. In some embodiments, an endogenous regulatory system can be employed such that T cell receptor—coupled IL-2 gene expression via the NFAT-AP-1 complex regulates expression of the therapeutic agent from the synthetic chromosome.

One example of such a circuit involves the use of Interferon Response Factor 9 (IRF9). The hSync can be engineered to include components of a circuit in which IRF9 binds Interferon Response Elements (ISREs) within the PD-1 gene, in order to make an interferon inducible system for promoting transcription of a PD-1 siRNA during T cell activation. In such a circuit, the regulated induction of siRNA production provides controlled silencing of the expression of the checkpoint PD-1 mRNA via the small interfering RNA. Thus, the presently disclosed system can be used to reverse the tumor immune escape mechanism.

3. Regulation of Gene Cassettes on the hSync Platform Utilizing Synthetic Programmable Transcriptional Regulators

Control of gene expression requires precise and predictable up and down spatiotemporal regulation. Modern molecular biology has taken advantage of naturally occurring gene expression systems that respond to developmental, environmental, and physiological cues and usurped evolved protein DNA binding domains to control expression of heterologous proteins. Naturally occurring bacterial systems such as those found in the DNA binding domains conferring tetracycline resistance (TetR), lactose metabolism (Lac), response to DNA damage (LexA), and cumate metabolism (CymR) have been adapted and engineered to control gene expression in mammalian cells. Likewise, naturally occurring animal and insect gene control systems such as heat shock control, hormone metabolism, and heavy metal metabolism have been engineered to control production of heterologous proteins in mammalian cells and transgenic animals.

Advances in synthetic biology bioengineering approaches have provided the tool sets required to produce synthetic transcriptional regulators. This approach builds upon adding known biological components such as DNA-binding domains from zinc finger proteins (ZF) or transcriptional activator-like proteins (TALE) and fusing them to transcriptional activation domains (AD) to interact with the RNA polymerase machinery and control gene expression. In turn, these synthetic regulators can be designed to bind to precise DNA sequences in gene promoter regions to either activate or repress gene expression as well as block transcription by terminating transcriptional elongation. Recently the bacterial native defense system, clustered regularly interspaced short palindromic repeat and Cas9 associated protein or CRISPR/Cas9, has been developed to circumvent the need to re-engineer DNA binding domains in ZF and TALE systems enabling targeting precise DNA sequences via RNA-DNA interactions dictated by the CRISPR/Cas9 system. For example, the guide element in the CRISPR can be designed to recognize specific DNA sequences and a mutated Cas9 nuclease domain (dCAS9) can be fused to effector domains such as repressors and activators to control transcription.

The hSync platform chromosome contains sufficient genetic bandwidth to control individual loaded genes or gene circuits with both engineered transcriptional regulators (e.g., TetR and CymR) or synthetic programmable transcriptional regulators. The hSync can be bioengineered to express multiple genes using DNA-binding domains (e.g. ZF and TALE) fused to activation domains or CRISPR/dCAS9 systems designed to target a variety of specific DNA sequences in promoters specified by a variety of guide RNAs.

### 4. Tracking Lineage and Spatiotemporal Analysis

The ability to define the status of a single cell within a diverse population has been impeded by the paucity of tools that have the capability to delineate multiple states within a single population. Synthetic chromosomes rationally engineered to contain select large genetic payloads without alteration of the host chromosomes significantly advance development of complex cell-based therapies. Such synthetic chromosomes can be used in vitro to screen the effect of exogenous stimuli on cell fate and/or pathway activation and in vivo to establish the effect of exposure to exogenous or endogenous signals on development with single cell resolution.

In some embodiments, the synthetic chromosome comprises a plurality of reporter genes driven by lineage-specific promoters.

In some embodiments, the lineage-specific promoters include promoters for Oct4 (pluripotency), GATA4 (endoderm), Brachyury (mesoderm), and Otx2 (ectoderm). In some embodiments, the synthetic chromosome comprises a plurality of reporter genes driven by damage- or toxin-responsive promoters. In some aspects, the promoters are promoters responsive to irradiation, heavy metals, and the like. In some embodiments, the present disclosure employs a synthetic chromosome comprising lineage-specific promoters linked to different fluorescent markers to provide readout for cell lineage fate determination.

In some embodiments, the synthetic chromosome may comprise an expression cassette to deliver a therapeutic agent such as a peptide, polypeptide or nucleic acid (natural or synthetic).

In some embodiments, the present invention provides a method of tracking transplanted cells bearing the synthetic chromosome in a live animal by tracking a reporter gene encoded on the synthetic chromosome in cells in the live animal.

The synthetic chromosome system described herein not only has the bandwidth to allow loading of large genomic regions, including endogenous regulatory elements, but also provides a stably maintained autonomously replicating and non-integrated chromosome which can serve as a cell-based biosensor for in situ analysis of single cell status within a diverse population in response to specific signals. The synthetic chromosome allows analysis of cell fate following exposure to exogenous stimuli and/or isolation of specific cells from a diverse population, with single cell resolution. Thus, in some embodiments, the present invention encompasses compositions and methods that allow one to perform single cell spatiotemporal analysis in response to differentiation cues, and/or to label transplanted cells to monitor their fate and function in a patient recipient. In some embodiments, the present disclosure provides an induced pluripotent stem cell comprising a synthetic chromosome comprising lineage specific promoters linked to different fluorescent markers to provide readout for cell lineage fate determination.

In some embodiments, human iPSCs are differentiated into embryoid bodies (EBs) and the EBs are monitored by confocal microscopy over time to confirm the presence of endo-, meso- and ectoderm lineages. Thus, the compositions and methods described herein provide a tool for single cell spatiotemporal analysis. In some embodiments, the present disclosure provides a method for differentiating into EBs induced pluripotent stem cells comprising a synthetic chromosome where the synthetic chromosome comprises lineage specific promoters, dissociating the embryoid bodies, and sorting and isolating cells of each lineage. In some embodiments, the present composition and methods allow isolation of cells of different lineages upon differentiation of pluripotent stem cells into EBs, dissociating the EBs, and sorting and isolating cells of each lineage. Microscopic imaging and quantitative RT-PCR can be used to quantify expression of lineage specific markers, and assess the degree of cell enrichment.

Additionally, the present invention provides an engineered synthetic chromosome utilizing mouse regulatory elements used to generate transgenic mice wherein the fate of single cells within a tissue and/or the organism is monitored following exposure to specific signals. Additionally, the present invention provides engineered synthetic chromosomes containing reporter genes driven by damage or toxins (e.g., irradiation, heavy metals, etc.) responsive promoters. The present invention further provides a human synthetic chromosome to be used to deliver stem cell-based therapeutics for regenerative or oncologic medicine, as well as containing reporters to allow tracking the transplanted cells.

5. Engineering Stem Cells, Reversing Senescence, Preventing Oxidative Stress and/or Inflammation, and Enhancing Reproductive Lifespan

Another use of the synthetic chromosome is in the engineering of stem cells for use in cell-based regenerative medicine. Inflammation is associated with aging via certain mediators of the senescence-associated secretory phenotype, IL-6 and IL-8. Klotho interacts with retinoic acid-inducible gene-1 (RIG-1) to inhibit RIG-1 dependent expression of IL-6 and IL-8, thereby delaying aging. In addition, evidence suggests that Klotho may delay aging by inhibiting the p53 DNA damage pathway.

Peroxisome proliferator-activated receptors gamma and delta are transcription factors that play a role in the anti-oxidant and anti-inflammatory cellular responses through activation of downstream gene expression including expression of Klotho. Crosstalk between these pathways leads to a complicated network of cellular factors contributing to cellular responses to limit damage and subsequent aging.

More general and poorly understood changes in global gene expression as a result of changes in chromatin conformation-through changes in expression in DNA methyltransferases, histone deacetylases and the non-histone high mobility group protein A2—have also been reported during aging. Changes in nuclear architecture also occur through alterations in maturation of nuclear lamin A from the prelamin A precursor.

Aging of somatic cells, including stem cells generally, is believed to be driven at least in part through attrition of chromosome ends, e.g., telomeres, as a consequence of imperfect end-replication and end-processing reactions. Germline and stem cells overcome these issues through the action of the specialized reverse transcriptase, telomerase, which adds DNA de novo to chromosome ends. However, numerous studies have shown that telomerase in stem cells is not sufficient to completely overcome telomere loss, ultimately limiting the number of divisions stem cells can undergo. Both differentiation potential and regenerative capacity of bone-marrow derived stem cells are reduced following serial transplantation; similarly, it has been demonstrated that telomeres are shorter in human allogeneic transplant recipients than in their respective donors, and both proliferative capacity and differentiation potential of circulating myeloid cells was significantly reduced in recipients as compared to their respective donors. Further, in addition to its essential role at chromosome ends, telomerase may also play a role in responding to oxidative stress. Production of reactive oxygen species increases as cells age-likely as a result of mitochondrial damage—and oxidative damage is thought to be a major driver of aging. In recent years it has been demonstrated that telomerase relocates to mitochondria when the cell is under oxidative stress, and increasing evidence suggests that relocation of the catalytic subunit of human telomerase, hTERT, to the mitochondria is essential in limiting oxidative damage. Damaged mitochondria result in higher production of reactive oxygen species leading to a dangerous cycle of ever increasing oxidative damage.

Additionally, expression of SIRT1, an NAD+-dependent protein deacetylase, is decreased in aged stem cells and it has been found that forced ectopic expression of SIRT1 can delay senescence of stem cells. SIRT1 has been shown to regulate oxidative stress and mediate the longevity effected by caloric restriction and has also been shown to regulate Wnt/β-catenin signaling that is important in the maintenance of stem cell pluripotency. Importantly, SIRT1 affects replicative senescence via upregulation of hTERT, thereby limiting oxidative damage to telomeres and mitochondria resulting in an extension of cellular replicative lifespan.

Nuclear factor erythroid 2-related factor (NFE2L2), a master regulator of the cellular oxidative stress response, is a transcription factor that activates antioxidant responsive element (ARE)-dependent genes encoding cellular redox regulators. In the absence of oxidative stress, NFE2L2 is bound to its inhibitor KEAP1 and targeted for proteasome mediated degradation. In the presence of stress, NFE2L2 is released from this complex and translocates to the nucleus to activate genes involved in the antioxidant response. NFE2L2 also positively regulates SIRT1 mRNA and protein through negative regulation of p53. In addition, NFE2L2 activates expression of subunits of the 20S proteasome. Aged cells contain high levels of oxidized proteins that can form aggregates resistant to degradation. Activation of the 20S proteasome via NFE2L2-dependent gene expression has also been found to result in extension of lifespan and stemness, presumably through proteasome-dependent degradation of oxidized proteins. Given the role of NFE2L2 in multiple pathways it is not surprising that forced expression of NFE2L2 results in improved differentiation potential and maintenance of stemness in stem cells.

In some embodiments, the present compositions and methods are useful in autologous transplantation for age-associated degenerative conditions such as osteoarthritis, in which cellular lifespan is limited and cells lose differentiation potential. For example, aging and cellular replicative lifespan are regulated via a series of interrelated pathways; in humans, expression of each of the hTERT, SIRT1 and NFE2L2 genes has been demonstrated to play a role in extending lifespan, perhaps through pathways that interact to regulate telomere damage and oxidative stress. Thus, these genes are excellent targets for manipulation to be used in rejuvenating stem cells, and for enhancing lifespan of a cellular therapeutic.

1. A mammalian synthetic chromosome comprising a nucleic acid sequence encoding a chemokine receptor.

2. A mammalian synthetic chromosome according to claim 1, wherein the chemokine receptor is selected from: CCR4, CCR6, CXCR4, CCR7, CXCR3, and CX3CR1.

3. A mammalian synthetic chromosome according to claim 1, wherein the chemokine receptor is CCR4 or CCR6.

4. A mammalian synthetic chromosome according to claim 1, wherein the chromosome comprises two or more nucleic acid sequences encoding two or more chemokine receptors, wherein the two or more chemokine receptors are the same or different.

5. A mammalian synthetic chromosome according to claim 4, wherein the nucleic acid sequences encode CCR4 and CCR6, respectively.

6. A mammalian synthetic chromosome according to claim 1, comprising one or more promoters independently controlling expression of one or more chemokine receptors.

7. A mammalian synthetic chromosome according to claim 1, wherein one or more promoters are inducible.

8. A mammalian synthetic chromosome according to claim 1, wherein one or more promoters are constitutive.

9. A mammalian synthetic chromosome according to claim 1, comprising two or more promoters, one of which is inducible and another is constitutive.

10. A mammalian synthetic chromosome according to claim 1, wherein the chemokine receptor is CCR6 and expression of CCR6 is inducible.

11. A mammalian synthetic chromosome according to claim 1, wherein the chemokine receptor is CCR4 and expression of CCR4 is constitutive.

12. A mammalian synthetic chromosome according to claim 1, comprising one or more insulators.

13. A mammalian synthetic chromosome according to claim 1, for use in cellular homing.

14. A mammalian synthetic chromosome for use in enhancing an therapeutic response in or in the vicinity of a target tissue by providing chemokine receptors expressed by cells carrying the chromosome.

15. A cell comprising a mammalian synthetic chromosome according to claim 1.

16. A cell according to claim 15, wherein the cell is a leukocyte.

17. A cell according to claim 15, for medical use, veterinary use, or diagnostic use.

18. A cell comprising a mammalian synthetic chromosome according to claim 15, for use in enhancing a response in or in the vicinity of a target tissue by providing chemokine receptors.

19. A composition comprising a mammalian synthetic chromosome according to claim 1, and an additive.

20. A composition comprising a cell according to claim 15, and an additive.
